(19) United States
(12) Patent Application Publication
(10) Pub. No.: US 2012/0226200 A1
(43) Pub. Date: Sep. 6, 2012

(54) METHODS OF STIMULATING TISSUE BASED UPON FILTERING PROPERTIES OF THE TISSUE

(75) Inventors: Timothy Andrew Wagner, Cambridge, MA (US); Uri Tzvi Eden, Somverville, MA (US)

(73) Assignee: HIGHLAND INSTRUMENTS, INC., Cambridge, MA (US)

(21) Appl. No.: 13/216,282

(22) Filed: Aug. 24, 2011

Related U.S. Application Data
(60) Provisional application No. 61/448,391, filed on Mar. 2, 2011.

(51) Int. Cl. A61H 1/00; A61F 7/00; A61N 5/06; A61N 1/36; A61N 7/00

(52) U.S. Cl. 601/2; 607/2; 601/1; 601/15; 607/45; 607/88; 607/96

(57) ABSTRACT

The invention generally relates to methods of stimulating tissue based upon filtering properties of the tissue. In certain aspects, the invention provides methods for stimulating tissue that involve analyzing at least one filtering property of a region of at least one tissue, and providing a dose of energy to the at least one region of tissue based upon results of the analyzing step.

### RELATED APPLICATION

The present application claims the benefit of and priority to U.S. provisional patent application Ser. No. 61/448,391, filed Mar. 2, 2011, the content of which is incorporated herein by reference in its entirety.

### GOVERNMENT SUPPORT

This invention was made with Government support under Grant Number R43NS062530 awarded by the National Institute of Neurological Disorders and Stroke (NINDS) of the National Institute of Health (NIH) and Contract No. W31P4Q-09-C-0117 awarded by Defense Advanced Research Projects Agency (DARPA). The Government has certain rights in this invention.

### FIELD OF THE INVENTION

The invention generally relates to methods of stimulating tissue based upon filtering properties of the tissue.

### BACKGROUND

There has been a rapid increase in the application of stimulation devices to treat a variety of pathologies, particularly neuropathologies. FDA approved therapies already include treatments for disorders such as Parkinson's disease, depression, and epilepsy, and the number of indications being explored is growing exponentially. Effective electromagnetic stimulation techniques alter the firing patterns of cells by applying electromagnetic energy to electrically responsive cells, such as neural cells. The stimulation may be applied invasively, e.g., by performing surgery to remove a portion of the skull and implanting electrodes in a specific location within brain tissue, or non-invasively, e.g., transcranial direct current stimulation or transcranial magnetic stimulation. Other forms of energy can also be used to stimulate tissue, both invasively and noninvasively.

In vitro biophysical models have been used to characterize interactions between the stimulation fields and the tissue and to assess location, magnitude, timing, and direction of the stimulation effects. Assessment of the applied fields is important for tailoring the effects of the individual stimulation modality to the intended outcome and to maximize the efficacy within safety constraints.

A problem with these biophysical models is that the models ignore fundamental physical processes occurring in tissue, particularly neural tissue, such as tissue filtering based on the frequency of the stimulation waveform. These filtering effects alter the predicted stimulatory waveforms in magnitude and shape and fundamentally impact the anticipated stimulation effects. Failure to account for tissue filtering properties has a clear implication on safety and dosing considerations for stimulation.

### SUMMARY

The invention generally relates to methods of stimulating tissue based upon filtering properties of the tissue. The invention recognizes that tissue filtering properties have an impact on all systems implementing stimulation waveforms with specific temporal dynamics tailored to an individual anatomical structure. For analyzing electromagnetic forms of stimulation, tissues can form a filtering network of capacitive, resistive, and/or inductive elements which cannot be ignored, as fields in the tissues can be constrained by these tissue electromagnetic properties. These tissue effects are important to consider while evaluating tissue response to electromagnetic fields and while developing electromagnetic-dosing standards for stimulation.

Furthermore, the invention provides methods to account for stimulation fields (based on tissue filtering data) that can be used to predict a tissue's response to stimulation, and thus methods of the invention are useful for optimizing stimulation waveforms used in clinical stimulators for a programmed stimulation effect on tissue. Methods of the invention predict stimulation electromagnetic field distribution information including location (target), area and/or volume, magnitude, timing, phase, frequency, and/or direction and also importantly integrate with membrane, cellular, tissue, network, organ, and organism models.

In certain aspects, the invention provide methods for stimulating tissue that involve analyzing at least one filtering property of a region of at least one tissue, and providing a dose of energy to the at least one region of tissue based upon results of the analyzing step. Exemplary filtering properties include anatomy of the tissue (e.g., distribution and location), electromagnetic properties of the tissue, cellular distribution in the tissue, chemical properties of the tissue, mechanical properties of the tissue, thermodynamic properties of the tissue, chemical distrubtions in the tissue, and/or optical properties of the tissue. Methods of the invention can be implemented during stimulation, after stimulation, or before stimulation (such as where dosing and filtering analysis could take place via simulation).

Any type of energy known in the art may be used with methods of the invention. In certain embodiments, the type of energy is mechanical energy, such as that produced by an ultrasound device. In certain embodiments, the ultrasound device includes a focusing element so that the mechanical field may be focused. In other embodiments, the mechanical energy is combined with an additional type of energy, such as chemical, optical, electromagnetic, or thermal energy.

In other embodiments, the type of energy is electrical energy, such as that produced by placing at least one electrode in or near the tissue. In certain embodiments, the electrical energy is focused, and focusing may be accomplished based upon placement of electrodes. In other embodiments, the electrical energy is combined with an additional type of energy, such as mechanical, chemical, optical, electromagnetic, or thermal energy.

In particular embodiments, the energy is a combination of an electric field and a mechanical field. The electric field may be pulsed, time varying, pulsed a plurality of time with each pulse being for a different length of time, or time invariant. The mechanical filed may be pulsed, time varying, or pulsed a plurality of time with each pulse being for a different length of time. In certain embodiments, the electric field and/or the mechanical field is focused.

The energy may be applied to any tissue. In certain embodiments, the energy is applied to a structure or multiple structures within the brain or the nervous system such as the dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord. In particular embodiments, the tissue is neural tissue, and the affect of the stimulation alters neural function past the duration of stimulation.

Another aspect of the invention provides methods for stimulating tissue that involve providing a dose of energy to a region of tissue in which the dose provided is based upon at least one filtering property of the region of tissue. Another aspect of the invention provides methods for stimulating tissue that involve analyzing at least one filtering property of a region of tissue, providing a dose of electrical energy to the region of tissue, and providing a dose of mechanical energy to the region of tissue, wherein the combined dose of energy provided to the tissue is based upon results of the analyzing step. Another aspect of the invention provides methods for stimulating tissue that involve providing a noninvasive transcranial neural stimulator, and using the stimulator to stimulate a region of tissue, wherein a dose of energy provided to the region of tissue is based upon at least one filtering property of the region of tissue.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 is a schematic showing an embodiment to analyze, control, or optimize energy dose based on tissue filtering.

FIG. 2 is a schematic showing an embodiment to analyze, control, or optimize energy dose based on tissue filtering where two separate energy dosing systems are connected between the source energy waveforms, but filtering and effects are analyzed on the fields independently.

FIG. 3 is a schematic showing an embodiment to analyze, control, or optimize energy dose based on tissue filtering where two energy systems' filtered energy waveforms combine in the tissues and filtering and its effects are examined on the combined energy.

FIG. 4 is a schematic showing an embodiment to analyze, control, or optimize energy dose based on tissue filtering where two energy systems provide combined energy to a tissue, where the filtering and its effects are examined on the combined energy.

FIG. 5 is a schematic showing different waveforms commonly used in DBS and/or TMS stimulation.

FIG. 6 is a graph showing Recorded Tissue Impedance Values within the Brain Stimulation Spectrum from 10 to 10,000 Hz (with comparison ex-vivo values from the literature). They demonstrate electromagnetic conductivity and permittivity values as a function of frequency.

FIG. 7 is a set of graphs showing transcranial magnetic stimulation (TMS) electromagnetic field example for the TMS 3 pulse (tri-phasic pulseform). The figure demonstrates the coil current, cortical current density waveforms, the composition of the cortical current densities on cortical surface at peak frequency, and the current composition as a function of time at the evaluation point, which ceneted 2.3 cm from the coil face. Root mean square (RMS) values were calculated across the pulse waveforms (defined as the square root of the average of the squares of the original values).

FIG. 8 is a set of graphs showing TMS Electric Field and Current Densities for the TMS 3 pulse evaluated along vectors approximately tangential and normal to the cortical surface.

FIG. 9 is a set of graphs showing Deep Brain Stimulation (DBS) Electromagnetic field example for the 600 charge balanced waveform (CB600).

FIG. 10 is a set of graphs showing Human Motor Neuron Thresholds as a function of the tissue properties examined for each of the sources and waveforms tested. TMS thresholds are evaluated at a location centered to figure-of-eight coil intersection 2.3 cm from coil face with a 25-turn air core copper coil, and the DBS thresholds at point 0.75 mm from the electrode contacts.

FIG. 11 is an example of simulation solutions based on artificially removing tissue capacitance compared to solutions including capacitive effects for a TMS example.

FIG. 12 is an example demonstrating electromechanical principles.

FIG. 13 is an example of current density magnitudes calculated in the cortex comparing tDCS and EMS.

### DETAILED DESCRIPTION

It is envisioned that the present disclosure may be used to guide, control, analyze, tune, optimize or predict energy fields during stimulation, accounting for their amplitude, volume (and/or area), direction, phase, transient (i.e., time), and/or spectral (frequency information) effects in the stimulated tissue, while simultaneously providing information about the targeted cell response, targeted network response, and/or systemic response. Furthermore this can be used to identify spectral content of relevance to specific neural responses and to thus tune the stimulation waveform to a desired effect.

The exemplary embodiments of the apparatuses and methods disclosed can be employed in the area of analyzing, predicting, controlling, and optimizing the dose of energy for neural stimulation, for directly stimulating neurons, depolarizing neurons, hyperpolarizing neurons, modifying neural membrane potentials, altering the level of neural cell excitability, and/or altering the likelihood of a neural cell firing (during and after the period of stimulation). Exemplary apparatuses for stimulating tissue are described for example in Wagner et al., (U.S. patent application numbers 2008/0046053 and 2010/0070006), the content of each of which is incorporated by reference herein in its entirety.

Likewise, methods for stimulating biological tissue may also be employed in the area of muscular stimulation, including cardiac stimulation, where amplified, focused, direction altered, and/or attenuated currents could be used to alter muscular activity via direct stimulation, depolarizing muscle cells, hyperpolarizing muscle cells, modifying membrane potentials, altering the level of muscle cell excitability, and/or altering the likelihood of cell firing (during and after the period of stimulation). Likewise, methods for stimulating tissue can be used in the area of cellular metabolism, physical therapy, drug delivery, and gene therapy. Furthermore, stimulation methods described herein can result in or influence tissue growth (such as promoting bone growth or interfering with a tumor). Furthermore, devices and methods can be used to solely calculate the dose of the fields, for non-stimulatory purposes, such as assessing the safety criteria such as field strengths in a tissue.

The embodiments outlined herein for calculating, controlling, tuning, and/or optimizing energy doses of stimulation can be integrated (either through feedback control methods or passive monitoring methods) with imaging modalities, physiological monitoring methods/devices, diagnostic methods/devices, and biofeedback methods/devices (such as those described in co-owned and co-pending U.S. patent application Ser. No. 13/162,047, the content of which is incorporated by reference herein in its entirety). The embodiments outlined herein for calculating/controlling energy doses of stimulation can be integrated with or used to control the stimulation source properties (such as number, material properties, position (e.g., location and/or orientation relative to tissue to be stimulated and/or other sources or components to be used in the stimulation procedure) and/or geometry (e.g., size and/or shape relative to tissue to be stimulated and/or other sources or components to be used in the stimulation procedure)), the stimulation energy waveform (such as temporal behavior and duration of application), properties of interface components (such as those outlined in (U.S. patent application number 2010/0070006) and for example position, geometry, and/or material properties of the interface materials), and/or properties of focusing or targeting elements (such as those outlined in (co-owned and co-pending U.S. patent application Ser. No. 13/169,288, the content of which is incorporated by reference herein in its entirety) and for example position, geometry, and/or material properties of the interface materials) used during stimulation.

The dose of energy(ies) can include the magnitude, position, dynamic behavior (i.e., behavior as a function of time), static behavior, behavior in the frequency domain, phase information, orientation/direction of energy fields (i.e., vector behavior), duration of energy application (in single or multiple sessions), type/amount/composition of energy (such as for electromagnetic energy, the energy stored in the electric field, the magnetic field, or the dissipative current component (such as could be described with a Poynting Vector)), and/or the relationship between multiple energy types (e.g., magnitude, timing, phase, frequency, direction, and/or duration relationship between different energy types (such as for example for an electromechanical energy (i.e., energy provided from mechanical field source, such as ultrasound device, and an electrical field source, such as an electrode) pulse, the amount of energy stored in an acoustic energy pulse compared with that stored in an electric pulse)). Dose of energy may be analyzed, controlled, tuned, and/or optimized for its impact on a cell, tissue, functional network of cells, and/or systemic effects of an organism.

The term tissue filtering properties refer to anatomy of the tissue(s) (e.g., distribution and location), electromagnetic properties of the tissue(s), cellular distribution in the tissue(s) (e.g., number, orientation, type, relative locations), mechanical properties of the tissue(s), thermodynamic properties of the tissue(s), chemical distributions in the tissue(s) (such as distribution of macromolecules and/or charged particles in a tissue), chemical properties of the tissue(s) (such as how the tissue effects the speed of a reaction in a tissue), and/or optical properties of the tissue(s) which has a temporal, frequency, spatial, phase, and direction altering effect on the applied energy. The term filtering includes the reshaping of the energy dose in time, amplitude, frequency, phase, type/amount/composition of energy, or position, or vector orientation of energy (in addition to frequency dependent anisotropic effects).

Filtering can result from a number of material properties that act on the energy, for example this includes a tissue's (and/or group of tissues'): impedance to energy (e.g., electromagnetic, mechanical, thermal, optical, etc.), impedance to energy as a function of energy frequency, impedance to energy as a function of energy direction/orientation (i.e., vector behavior), impedance to energy as a function of tissue position and/or tissue type, impedance to energy as a function of energy phase, impedance to energy as a function of energy temporal behavior, impedance to energy as a function of other energy type applied and/or the characteristics of the other energy type (such as for a combined energy application where an additional energy type(s) is applied to modify the impedance of one tissue relative to other energy types that are applied), impedance to energy as function of tissue velocity (for tissue(s) moving relative to the energy and/or the surrounding tissue(s) moving relative to a targeted tissue), impedance to energy as a function of tissue temperature, impedance to energy as a function of physiological processes ongoing in tissue(s), impedance to energy as a function of pathological processes ongoing in tissue(s), and/or impedance to energy as a function of applied chemicals (applied directly or systemically).

Filtering can further be caused by the relationship between individual impedance properties to an energy or energies (such as for example the relationship that electrical conductivity, electrical permittivity, and/or electrical permeability have to each other). This can further include the velocity of propagation of energy in the tissue(s), phase velocity of energy in the tissue(s), group velocity of energy in the tissue(s), reflection properties to energy of the tissue(s), refraction properties to energy of the tissue(s), scattering properties to energy of the tissue(s), diffraction properties to energy of the tissue(s), interference properties to energy of the tissue(s), absorption properties to energy of the tissue(s), attenuation properties to energy of the tissue(s), birefringence properties to energy of the tissue(s), and refractive properties to energy of the tissue(s). This can further include a tissue(s'): charge density (e.g., free, paired, ionic, etc.), conductivity to energy, fluid content, ionic concentrations, electrical permittivity, electrical conductivity, electrical capacitance, electrical inductance, magnetic permeability, inductive properties, resistive properties, capacitive properties, impedance properties, elasticity properties, stress properties, strain properties, combined properties to multiple energy types (e.g., electroacoustic properties, electrothermal properties, electrochemical properties, etc), piezoelectric properties, piezoceramic properties, condensation properties, magnetic properties, stiffness properties, viscosity properties, gyrotropic properties, uniaxial properties, anisotropic properties, bianisotropic properties, chiral properties, solid state properties, optical properties, ferroelectric properties, ferroelastic properties, density, compressibility properties, kinematic viscosity properties, specific heat properties, Reynolds number, Rayleigh number, Damkohler number, Brinkman number, Nusselt Schmidt number, number, Peclet number, bulk modulus, Young's modulus, Poisson's ratio, Shear Modulus, Prandtl number, Adiabatic bulk modulus, entropy, enthalpy, pressure, heat transfer coefficient, heat capacity, friction coefficients, diffusivity, porosity, mechanical permeability, temperature, thermal conductivity, weight, dimensions, position, velocity, acceleration, shape, convexity mass, molecular concentration, acoustic diffusivity, and/or coefficient of nonlinearity.

Filtering can occur at multiple levels in the processes. For example with multiple energy types filtering can occur with the individual energies, independent of each other (such as where acoustic and electrical energy are applied to the tissue at separate locations and the fields are not interacting at the sites of application), and then filtering can occur on the combined energies (such as where acoustic and electrical energy interact in a targeted region of tissue).

Furthermore, any material and/or sub-property in a focusing element, interface element, and/or component(s) of the energy source element that can actively or passively alter the energy field properties of stimulation can also be accounted for in the dosing procedures explained herein (including any space, fluid, gel, paste, and material that exists between the tissue to be stimulated and the stimulation energy source). For example, methods of the invention can also account for: lenses (of any type (e.g., optical, electromagnetic, electrical, magnetic, acoustic, thermal, chemical, etc)); using waveguides; using fiber optics; phase matching between materials; impedance matching between materials; using reflection, refraction, diffraction, interference, and/or scattering methods between materials.

In certain embodiments, methods of the invention can be accomplished with computers, mobile devices, dedicated chips or circuitry (e.g., in control system of stimulator or integrated imaging device or external dose controller), remote computational systems accessed via network interfaces, and/or computational devices known in the art. Methods of the invention can be accomplished with software for performing various computer-implemented processing operations such as any or all of the various operations, functions, and capabilities described herein. In certain embodiments, the processing operations include accessing a database of source, tissue, organ, network, organism, and/or cellular properties which can be stored in any form of computer storage.

The term “computer-readable medium” is used herein to include any medium capable of storing data and/or storing or encoding a sequence of computer-executable instructions or code for performing the processing operations described herein. The media and code can be those specially designed and constructed for the purposes of the invention, or can be of the kind well known and available to those having ordinary skill in the computer and/or software arts. Examples of computer-readable media include computer-readable storage media such as: magnetic media such as fixed disks, floppy disks, and magnetic tape; optical media such as Compact Disc-Read Only Memories (“CD-ROMs”) and holographic devices; magneto-optical media such as floptical disks; memory sticks “flash drives” and hardware devices that are specially configured to store and execute program code, such as Application-Specific Integrated Circuits (“ASICs”), Programmable Logic Devices (“PLDs”), Read Only Memory (“ROM”) devices, and Random Access Memory (“RAM”) devices. Examples of computer-executable program instructions or code include machine code, such as produced by a compiler, and files containing higher level code that are executed by a computer using an interpreter. For example, an embodiment of the invention may be implemented using Java, C++, or other programming language and development tools. Additional examples of instructions or code include encrypted code and compressed code. Other embodiments of the invention can be implemented in whole or in part with hardwired circuitry in place of, or in combination with, program instructions/code.

The software can run on a local computer or a remote computer accessed via network connections. The computer may be a desktop computer, a laptop computer, a tablet PC, a cellular telephone, a Blackberry, or any other type of computing device. The computer machine can include a CPU, a ROM, a RAM, an HDD (hard disk drive), an HD (hard disk), an FDD (flexible disk drive), an FD (flexible disk), which is an example of a removable recording medium, a display, an I/F (interface), a keyboard, a mouse, a scanner, and a printer. These components are respectively connected via a bus and are used to execute computer programs described herein. Here, the CPU controls the entire computer machine. The ROM stores a program such as a boot program. The RAM is used as a work area for the CPU. The HDD controls the reading/writing of data from/to the HD under the control of the CPU. The HD stores the data written under the control of the HDD. The FDD controls the reading/writing of data from/to the FD under the control of the FDD. The FD stores the data written under the control of the FDD or causes the computer machine to read the data stored in the FD. The removable recording medium may be a CD-ROM (CD-R or CD-RW), an, a DVD (Digital Versatile Disk), a memory card or the like instead of the FD. The display displays data such as a document, an image and functional information, including a cursor, an icon and/or a toolbox, for example. The display may be a CRT, a TFT liquid crystal display, or a plasma display, for example. The I/F may be connected to the network such as the Internet via a communication line and is connected to other machines over the network. The I/F takes charge of an internal interface with the network and controls the input/output of data from/to an external machine. A modem or a LAN adapter, for example, may be adopted as the I/F. The keyboard includes keys for inputting letters, numbers and commands and is used to input data. The keyboard may be a touch-panel input pad or a numerical keypad. The mouse is used to move a cursor to select a range to move or change the size of a window. A trackball or joystick, for example, may be used as a pointing device if it has the same functions.

Components used with methods of the invention are fabricated from materials suitable for a variety medical applications, such as, for example, polymerics, gels, films, and/or metals, depending on the particular application and/or preference. Semi-rigid and rigid polymerics are contemplated for fabrication, as well as resilient materials, such as molded medical grade polyurethane, as well as flexible or malleable materials. The motors, gearing, electronics, power components, electrodes, and transducers of the method may be fabricated from those suitable for a variety of medical applications. The method according to the present disclosure may also include circuit boards, circuitry, processor components, etc. for computerized control. One skilled in the art, however, will realize that other materials and fabrication methods suitable for assembly and manufacture, in accordance with the present disclosure, also would be appropriate.

The following discussion includes a description of the components and exemplary methods for dosing the energy fields in biological tissues and the resulting tissue effects/response in accordance with the principles of the present disclosure. Alternative embodiments are also disclosed. Methods are disclosed for controlling the dosing of energy fields, such as electromagnetic (e.g., electrical, magnetic energies), chemical, mechanical, thermal, optical, and/or combined energy fields (e.g. electromechanical (i.e., with electrical energy and mechanical energy)). Reference will now be made in detail to the exemplary embodiments of the present disclosure illustrated in the accompanying figures.

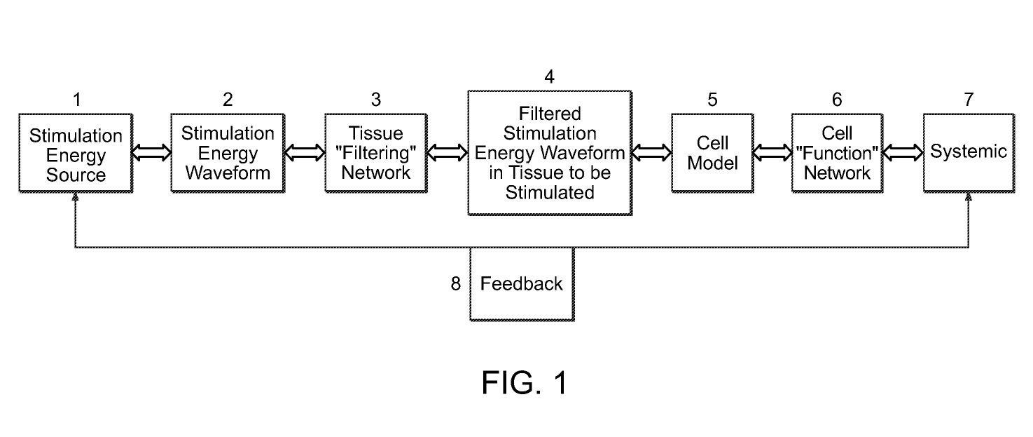

FIG. 1 shows an embodiment of methods of the invention. Electromagnetic fields (e.g., electrical fields, magnetic fields, electric current density fields (e.g., ohmic currents, displacement currents), magnetic flux density fields, and electric displacement fields) are created in the tissue(s) to be stimulated by an electric stimulation source. Electrically responsive cells and tissue can be effected by the electromagnetic energy that travels in the tissue, in or surrounding the cells. This can impact a network and ultimately be examined in terms of its impact on the organism stimulated (from cell to tissue to network (and/or to an organ, such as for example when one is stimulating cells of the heart) to organism). In order to determine, guide, control, optimize, tune, or predict the characteristics of the electromagnetic field distribution (e.g., direction, magnitude, frequency, phase, and timing) in the tissue(s) to be stimulated one must account for driving source of the electromagnetic fields during stimulation (such as the transducer location/position, transducer geometry, transducer material properties, and electromagnetic driving parameters of the fields (such as their amplitude and timing)), the electromagnetic properties of the tissue to be stimulated (such as the electromagnetic impedance of the tissue to be stimulated as a function of the power spectral content of the stimulation energy waveforms and the tissue's anatomical distribution (positions, distribution, shape of tissue(s) relative the stimulator source)), the targeted cells and their properties (such as distribution, orientation, level of electrical excitability), the functional network the cells are part of (such as network connections, inputs, and outputs), and the effect on the system.

During stimulation an electromagnetic energy source (box 1), such as an electrode or magnetic coil, applies an electromagnetic energy pulse(s) or continuous wave of electromagnetic energy (box 2) to tissue to be stimulated which can act as a filter to the energy (box 3) resulting in a filtered energy pulse or continuous wave of energy (box 4) in the tissue to be stimulated. The filtered electromagnetic energy stimulates a cell (box 5) in the tissue, such as a neuron, and ultimately affects a network of cells (box 6), which is responsible for some function or function(s), such as the reward system in the brain of an organism (e.g. mesolimbic pathway), and lead to systemic effects in the organism that is stimulated (box 7), such as in output behavior of the organism being stimulated (e.g. one could interfere with a craving response if an organism's reward system was stimulated). This process can be controlled and/or monitored via a feedback mechanism (box 8), active or passive, which modifies any of the elements of the dosing procedure based on information from imaging modalities, biofeedback, physiological measures, and/or other measures, such as those exemplified in co-owned and copending U.S. patent application Ser. No. 12/162,047.

While the methods herein are exemplified in an inclusive linear manner, each of the individual components (or subsets of the components in any permutation or group) can be isolated and analyzed through the methods outlined herein. For example, one could guide dosing based on just an energy pulse field (box 2), the tissue filtering network (box 3), the resulting filtered energy pulse (box 4), and a model of a cell (box 5) to analyze the response of a cell to individual electrical signals (such as to optimize a DBS waveform to a particular cell type with the least amount of energy used). As another example, one could guide dosing based on just an energy pulse field (box 2), the tissue filtering network (box 3), and the resulting filtered energy pulse (box 4) to assess the total amount and composition of the energy placed in a tissue (such as to optimize a transcranial electrical stimulation waveform with the safest level of energy in a tissue). Furthermore, the filtering network and the cell function network are separate functional entities (although comprised of the some or all of the same subcomponents), and their purpose in the method(s) and/or device(s) exemplified herein is different. As used herein, the filtering network pertains to filtering applied energy, while the functional cell network pertains the integrated function of cells for physiological function.

Turning now to box 1 of FIG. 1, the electromagnetic stimulation source can be a voltage source, current source, magnetic field source, electric field source, and/or any of these in combination with any means to modify these fields. It can be a an electrode used during Transcranial Direct Current Stimulation (TDCS), Transcranial Electrical Stimulation (TES), Transcranial Alternating Current Stimulation (TACS), Cranial Electrical Stimulation (CES), deep brain stimulation (DBS), microstimulation, pelvic floor and/or nerve stimulation, gastric stimulation, spinal cord stimulation (SCS), or vagal nerve stimulation (VNS). It can be a coil used for or Transcranial Magnetic Stimulation (TMS). The energy source can also be charged particle(s) or locations of charged particles (such as electric charge densities (which can for instance be injected into tissues), magnetic charge densities, ions, charged macromolecules, charged membranes, charged channels, and/or charged pores). It can further be evaluated as an electromechanical source (i.e., with combined electrical and mechanical field sources, such as an electrode(s) and an ultrasound source), where the electrical effects of the stimulation are analyzed as the primary effect.

One can also account for the circuit and control circuitry that feeds the source, and energy that might be fed into the source, such as a voltage or current signal. Any source parameter can be accounted for while determining, controlling, tuning, and/or optimizing the electromagnetic dose, including for example the source geometry, source position (location and orientation relative to stimulated tissue), source number, source material properties, source temperature, and/or source kinematics (if moving). For example, one could tune the geometry and placement location/orientation of a surface electrode on the scalp used for transcranial electric stimulation to target specific neurons in the brain based on the dosing procedure herein. For example, one could calculate the dose based on the full system of FIG. 1, leaving the electrode shape and placement as variables in the dosing calculation, which can be optimized via calculations based on computational iterations that are focused on the response of specifically targeted neural cells (which for example could have key membrane features, geometries, or orientations that the dose of energy are tuned for). Alternatively, one could actively adapt the geometry and placement location/orientation of a surface electrode on the scalp used for transcranial electric stimulation based on feedback and/or one could integrate these methods with stereotactic targeting equipment (with or without feedback) to control and direct stimulation. As another example, one could use the dosing methods outlined herein for source optimization, and characterize the individual source parameter(s) one is interested in accounting for in the analysis. For instance, in designing an optimum transducer device one could analyze the affects of different transducer materials and the transducer shape while determining the electromagnetic dose effects on neural cells.

Turning now to box 2 of FIG. 1, the stimulation source waveform can be any electromagnetic field such as magnetic fields, current density fields (e.g., ohmic and/or displacement currents), and/or an electric fields (which can all be accounted for via magnetic or electrical potentials), which are driven by energy inputs such as an electrical current or voltage waveform driving the field generation (or any energy type that can be converted to electrical energy for the generation of an electromagnetic field, such as chemical energy from a battery or mechanical energy from an electromechanical machine).

The electromagnetic energy is also a function of the source, including for example the source geometry, source position (location and orientation relative to stimulated tissue), source number, source material properties, source temperature, and/or source kinematics (if moving) and energy driving or fed into the source (for instance energy from a battery source and circuit controller, such as a current or voltage signal driving an DBS electrode implanted in the brain). One can account for individual electromagnetic pulses (or continuous waves) and evaluate their spectral frequency behavior, temporal behavior, amplitude, phase information, vector behavior (i.e., direction). Pulse trains can additionally be analyzed, including parameters such as pulse frequency, inter-pulse interval, individual pulse shape history, individual pulse interdependency. For example, one could tune the spectral content of applied electromagnetic pulses (including amplitude and dynamic behavior) and the time period between the application of multiple pulses applied with an electrode implanted in the brain to stimulate neurons with a specific timing pattern based on the integrated dosing procedure herein.

Turning now to box 3 of FIG. 1, the filtering network of the tissue to be stimulated can include individual cells, tissues, groups of tissues, and/or groups of cells and individual filtering properties or groups of filtering properties. One can account for this filtering network with a computational model of the tissues, depicting their geometry and distribution relative to the stimulation energy source and applied stimulation energy waveforms. One needs to account for the effects of the tissue parameters on the applied energy field(s) (box 2), and specifically the filtering effects the tissues/cells can have on the electromagnetic energy in the tissue (i.e., those parameters that effect the electromagnetic fields spectral frequency behavior, temporal behavior, amplitude, phase information, vector behavior).

Turning now to box 4 of FIG. 1. Ultimately the tissue filtering network (box 3) alter the applied electromagnetic energy (box 2), such that it is filtered in the tissue network. Thus, this filtered electromagnetic energy (box 4) in the tissue can be altered in spectral frequency behavior, temporal behavior, amplitude, phase information, vector behavior (i.e., direction), and or type/amount/composition of energy as functions of position, time, tissue, direction, phase, and/or any of the properties of the tissue filtering network as elaborated above, whereby individual energy pulses, continuous waves, and/or pulse trains can be affected.

This filtered electromagnetic energy (box 4) is what stimulates the cells in the tissue, and this energy also can impact the tissue itself (and/or the active or passive response of the tissue). For instance this filtered electromagnetic energy (box 4) in the tissue can be evaluated for its impact on tissue in terms of safety guidelines, such as looking at type/amounts of energy that are carried as displacement currents compared to ohmic currents, or to looks at the amount of energy that is dissipated in resistive processes that can raise tissue temperature, or to analyze the electromagnetic energy to determine how it drives electrochemical processes in the tissue. Ultimately this filtered electromagnetic energy can stimulate the tissue (and the cells within the tissue).

Turning now to box 5 of FIG. 1, which is a cell (box 5) which is located in the tissue filtering network (box 3) and exposed to the filtered electromagnetic energy (box 4), which was generated by the electromagnetic energy source (box 1) in the form of the source electromagnetic energy (box 2). The cell(s) can be any type of biological cell (e.g., cells of the muscle skeletal system, cells of the cardiac system, cells of the endocrine system, cells of the nervous system, cells of the respiratory system, cells of the immune system, cells of the digestive system, cells of the renal system, benign cells, malignant cells, pathological cells, healthy cells, etc), such as for example a cell or cells of the nervous system (e.g., neurons, glial cells, astroglia, etc). The filtered electromagnetic energy can interact with the cell and stimulate it (the energy can be in, on, and/or surrounding the cell). For example the electromagnetic energy can be used for directly stimulating neurons, depolarizing neurons, hyperpolarizing neurons, modifying neural membrane potentials, altering the level of neural cell excitability, and/or altering the likelihood of a neural cell firing during and after the period of stimulation.

One could use this method to analyze, predict, tune, optimize, or control cellular response to stimulation and one could examine a cell's (or individual subcomponents of the cell such as the cell body or the axon in the case of a neuron): geometry, shape, size, orientation, membrane characteristics (e.g., geometry, shape, size, channel concentrations, membrane impedance, membrane composition (e.g., for an axon whether it is mylenated or not)), dynamic characteristics (such as refractory periods), intracellular fluid composition, ionic concentrations (inside the cell and surrounding the cell), response to other cell(s) (such as inputs received from other cells), response to chemical transmitters (such as neurotransmitters), membrane channel characteristics (e.g., geometry, size, shape, conductance, charge characteristics, activity dynamics, refractory times), membrane pore characteristics, fluid flow dynamics surrounding the cell, mechanical movement surrounding the cell, velocity or position relative to the applied or filtered electromagnetic energy (or source), membrane channels resistance to specific ionic flow, ionic channel conductances, and/or charged proteins in or on cell (such as embedded in a cell's membrane).

This could further include a cell's: membrane, pores, vesicle, extracellular scaffolding, cytoskeleton, organelles, trans membrane proteins, synaptic endplates, synaptic vesicles, cellular transduction components, proteins, macromolecule, small molecules, gap junctions, enzymes, lipid, ribosome, transduction elements, transcription elements, translation elements, intercellular junctions, aptotic triggers, cascade signaling elements, stretch receptors, cellular receptors, binding sites, growth factors, regulatory proteins, stem cell factors, differentiation factors, transmembrane transporters, energy transduction elements, membrane pumps, transmembrane proteins, transport protiens, transporter carrier protiens, secretory protiens, binding protiens, docking elements, transporter, desmosomes, binding structures. Furthermore one could account for activity in the medium that surrounds the cell (such as the extracellular fluid or ionic double layers around cellular membranes).

One could select from these elements and build a model of the cell that is responsive to the electromagnetic energy that is applied, such as in generating a neural model that captures the impedance of the cell membrane and/or individual ions channel as a function (in time, space, and/or frequency) of the filtered electromagnetic energy in the surrounding tissue. Furthermore one can include outputs in the neural model that describe the voltage change and ionic flows along the neural membrane as a function of the applied electromagnetic energy to predict the neuron's electrochemical response to stimulation. The cell models can be used to capture one energy effect on the cell's response to another energy type, and/or the cell can be modeled where it responds in a different physical manner than in the type of energy that is applied (e.g., for a electromagnetic stimulation the cell can be modeled to respond in a electromagnetic, mechanical, chemical, optical, and/or thermal manner); these ideas can also be applied to network, organ, and/or systemic effect models.

Turning now to box 6 of FIG. 1, which is a functional network (box 6) of connected cells (box 5) which can be part of the tissue filtering network (box 3) that filters the applied electromagnetic energy (box 2), or larger than the area that contains the tissue that was directly targeted via the electromagnetic energy (i.e., the stimulation can impact entire networks beyond the target of the initial energy via connections in between the individual cells and components of the network (such as for example in a neural network, the initial stimulation energy could be directly focused on a group of cells in the motor cortex of a brain, but also impact subcortical structures, such as in the thalamus, due to transynaptic connections)).

By examining this network and the stimulated cells (those directly affected by the energy and the connected components) one can ultimately predict the systemic effect (box 7) of stimulation, such as for example where one is focusing electromagnetic energy on the brain's dorsal lateral prefrontal cortex (DLPFC) to excite the neural targets, with either a facilitatory or inhibitory signal, one can affect the emotional network of the brain and ultimately the emotional state of a subject being stimulated (this can be analyzed through direct effects on the DLPFC or through direct or indirect connections to other locations in the brain that process emotion, such as the amygdala (e.g., the systemic effect (box 7) can either be analyzed through the cells (box 5), the direct neural targets in the DLPFC, or through analyzing the functional network as a whole or in part (box 6)).

The entire method of dosing could be connected through feedback (box 8) to analyze, optimize, tune, or control the method, where in FIG. 1, (box 8) connects the analysis of effect with the stimulation source (box 1). This dosing process can be controlled and/or monitored via a feedback mechanism (box 8), active or passive, which modifies any of the elements of the dosing procedure based on information from imaging modalities, biofeedback, physiological measures, simulation results (based on the dosing/filtering method detailed herein), and/or subcomponent analysis, all of which are further described in co-owned and co-pending U.S. patent application Ser. No. 13/162,047.

This feedback can be integrated with an automated controller or can be based on user control, and implemented during stimulation, post stimulation, and/or pre-stimulation. Although this feedback method is demonstrated to connect the full dosing process, it should be noted that this is provided as an example to demonstrate that any of the components of the process could be interconnected, for instance feedback can be established between individual components of the process or within subsets of the process if the full dosing process is not analyzed. Feedback can be based on the connections between individual components, such as for example a method to record and analyze the effect of neural stimulation which is integrated with a controller which changes the timing of electromagnetic energy provided for stimulation based on the recorded affect of stimulation or with integrated systems such as where one device controls the electromagnetic energy source and records and analyzes the neural effect. Feedback can be implemented with a computational device that provides control and or analysis for each of the individual aspects of the process (where a feedback driven controller can adjust the parameters of the source (box 1) or the source electromagnetic energy (box 2) or even the filtering network (box 3), such as for example could be done with a second type of energy that is used to alter the impedance of the tissue in the presence of an electromagnetic field (as can also be done for the generation of additional electromagnetic energy where a second energy type is converted to electromagnetic energy (such as by boosting the currents applied, as described for example in U.S. patent application number 2008/0046053)).

To develop a computational model or device to assess, control, tune, and/or optimize the stimulation dose and/or stimulation process, one can model each of the individual components of the stimulation process or the system as a whole or in part (through integrated models of the system). One can model the electromagnetic source (box 1) and/or the electromagnetic source energy fields (box 2) with a software package, based on methods, such as computational or analytical methods (such as for example those methods described in Fields, Forces, and Flows in Biological Systems by Alan J. Grodzinsky (2011); Electromagnetic Field Theory: A Problem Solving Approach by Markus Zahn (2003); Electromechanical Dynamics, Parts 1-3: Discrete Systems/Fields, Forces, and Motion/Elastic and Fluid Media by Herbert H. Woodson and James R. Melcher (1985); Electromagnetic Fields and Energy by Hermann A. Haus and James R. Melcher (1989); Continuum Electromechanics by James R. Melcher (1981)), separation of variable methods (such as for example those described in “Numerical Techniques in Electromagnetics” by Sadiku, 2009), series expansion methods (such as for example those described in “Numerical Techniques in Electromagnetics” by Sadiku, 2009), finite element methods (such as for example those described in “Numerical Techniques in Electromagnetics” by Sadiku, 2009; “The Finite Element Method in Electromagnetics” by Jian-Ming Jin (May 27, 2002); “Electromagnetic Modeling by Finite Element Methods (Electrical and Computer Engineering”) by João Pedro A. Bastos and Nelson Sadowski (Apr. 1, 2003); “The Least-Squares Finite Element Method: Theory and Applications in Computational Fluid Dynamics and Electromagnetics (Scientific Computation)” by Bo-Nan Jiang (Jun. 22, 1998)), variational methods (such as for example those described in “Numerical Techniques in Electromagnetics” by Sadiku, 2009; “Variational methods for solving electromagnetic boundary value problems: Notes on a series of lectures given by Harold Levine under the sponsorship of the Electronic Defense Laboratory” by Levine, 1954; “Electromagnetic And Acoustic Scattering Simple Shapes” by Piergiorgio L. Uslenghi, Thomas B. Senior and J. J. Bowman, 1988), finite difference methods (e.g., in time domain, frequency domain, spatial domain, etc) (such as for example those described in “Numerical Techniques in Electromagnetics” by Sadiku, 2009; “Finite Difference Methods for Ordinary and Partial Differential Equations: Steady-State and Time-Dependent Problems (Classics in Applied Mathematics)” by Randall J. LeVeque, 2007; “Numerical Solution of Partial Differential Equations: Finite Difference Methods (Oxford Applied Mathematics & Computing Science Series)” by G. D. Smith, 1986; “Numerical Partial Differential Equations Finite Difference Methods (Texts in Applied Mathematics)”, by J. W. Thomas, 2010), moment methods (such as for example those described in “Numerical Techniques in Electromagnetics” by Sadiku, 2009; “Generalized Moment Methods in Electromagnetics: Formulation and Computer Solution of Integral Equations” by J. J. H. Wang, 1991; “The Method of Moments in Electromagnetics” by Gibson, 2007), matrix methods (such as for example those described in “Numerical Techniques in Electromagnetics” by Sadiku, 2009), Monte Carlo methods (such as those described in “Numerical Techniques in Electromagnetics” by Sadiku, 2009; Monte Carlo Methods for Electromagnetics by Matthew N. O. Sadiku (Apr. 9, 2009); “Monte Carlo Methods in Fuzzy Optimization (Studies in Fuzziness and Soft Computing)” by James J. Buckley and Leonard J. Jowers (Nov. 23, 2010)), perturbation methods (such as for example those described in “Perturbation Methods (Wiley Classics Library)” by Ali Hasan Nayfeh (Aug. 3, 2000); “Perturbation Methods (Cambridge Texts in Applied Mathematics)” by E. J. Hinch (Oct. 25, 1991)), genetic algorithm based methods (such as those used for optimization as described in “Genetic Algorithms in Electromagnetics” by Haupt and Warner, 2007; or “Electromagnetic Optimization by Genetic Algorithms” by edited by Rahmat-Samii and Michielessen, 1999), iterative methods (such as for example those described in “Iterative and Self-Adaptive Finite-Elements in Electromagnetic Modeling” by Magdalena Salazar-Palma, Tapan K. Sarkar, Luis-Emilio Garcia-Costillo and Tammoy Roy (September 1998)), and/or optimization methods (such as for example those described in “Optimization Methods in Electromagnetic Radiation (Springer Monographs in Mathematics)” by Thomas S. Angell and Andreas Kirsch (Jul. 1, 2011); “Optimization and Inverse Problems in Electromagnetism” by Marek Rudnicki and Slawomir Wiak (Dec. 23, 2010)) written in code with languages such as C, C++, Matlab, Mathematica, Fortran, C Sharp, Basic, Java, and/or other programming languages and/or with the use of commercial electromagnetic modeling packages such as Ansoft/ANSYSY Maxwell, COMSOL, and/or IBM Electromagnetic Field Solver Suite of Tools.

To determine the tissue/cellular filtering effects (box 3) on the applied electromagnetic energy (box 2), one can use an MRI, or any mapping of the tissue space (such as PET, MRI, X-Ray, CAT scan, Diffusion Spectrum Imaging (DSI), or Diffusion Tensor Imaging (DTI)), as a basis to generate a computer aided design (CAD) renderings of the tissue(s) to be stimulated. Additionally, one does not always need a medical imaging rendering of the tissues to determine or guide dosing, but one can also use prototypical shapes (e.g., simple geometries representing the tissue, or generic models to represent typical tissues (such as a simplified sphere model to represent the human brain for calculating the dose of electromagnetic energy for brain stimulation)). The mapping of tissue space will serve as the basis for an electromagnetic computational model of the tissue(s) to be stimulated. The mapping will provide geometry (tissue shapes) and distribution (relative placement of multiple tissues to each other) information relative to the electromagnetic energy source (box 1) and/or electromagnetic energy fields (box 2) that are used for stimulation. In certain embodiments, this process can be completed with just prototypical source energy fields (box 2), and the source components can be ignored (box 1), by modeling the impact of placing tissue in the path of a prototypical electromagnetic energy field. For instance, placing the brain in the path of a specific time changing magnetic field. One will assign properties to the mapped tissues that impact the filtering, thus defining the filtering network (box 3), for the computational calculation by mapping the individual tissue filtering properties (such as frequency dependent conductivity, permittivity, and permeability) onto the tissue space of the computational model to solve for the resulting filtering electromagnetic fields. The tissue filtering properties can be determined in advance through invasive or noninvasive methods, or during stimulation with invasive or noninvasive methods (such as noninvasive tissue spectroscopy).

One can also forego mapping the tissue space and reduce the filtering effects to a simplified equation to capture the tissue filtering effects on the applied energy. For example, one can represent the model of a group of tissues by a filtering network that can be reduced to a simple equation at the targeted site of stimulation, such as calculating the total filtering that takes place between a target site based on the number, dimensions, and filtering characteristics of tissues that are in between the stimulation energy source and the targeted cells (such as reducing multiple tissues to their complex impedances, thereby generating a filtering circuit, which can be reduced to a simplified equation with circuit analysis (such as that seen in Electric Circuits (9th Edition) (MasteringEngineering Series) by James W. Nilsson and Susan Riedel (2010)))).

To solve for the filtered stimulation fields (box 4) in the tissue one could use any known computational solution method. Exemplary methods include analytical and computational methods, separation of variable methods, series expansion methods, finite element methods, variational methods, finite difference methods (e.g., in time domain, frequency domain, spatial domain, etc), moment methods, matrix methods, Monte Carlo methods, perturbation methods, genetic algorithm based methods, iterative methods, and/or optimization methods written in code with languages such as C, C++, Matlab, Mathematica, Fortran, C Sharp, Basic, Java, or other programming languages and/or with the use of commercial electromagnetic modeling packages such as Ansoft/ANSYSY Maxwell, COMSOL, and/or IBM Electromagnetic Field Solver Suite of Tools.

Next, one can calculate the impact of the filtered electromagnetic energy (box 4) on the tissues that are being stimulated, such as with safety thresholds (such as for example in analyzing the breakdown of the energy in the tissue such as comparing ohmic and displacement currents) or by examining the tissue as an active average of the cells which comprise it (such as for example determining the effects of stimulation on the excitability of the tissue such as through the average makeup and response of the cells which serve as the building blocks of the tissue).

The next step in a computational process includes determining the impact of the filtered electromagnetic energy (box 4) on the cell(s) (box 5) in the tissues that are stimulated. Computationally one can develop a model of the response of the cell to the electromagnetic energy, such as for example by developing a multi-compartment model of a neuron that was being stimulated. The model of the cell could model any component of the cell which is responsive to the filtered electromagnetic energy (such as developing a multi-compartment model of the cell that includes a membrane comprised of resistive and capacitive components (these components could be frequency or time dependent) for each of the analyzed elements of a cell (such as for example an axon, cell, body, and dendrites in a neuron), half cell potentials due to ion distributions, and voltage gated channels where their resistance to ion flow is dependent on the electromagnetic energy in the tissue surrounding the cell (the channels could have a frequency dependence, time dependence, orientation dependence, or any computationally and/or biologically relevant characteristic)).

This dosing calculation allows one to determine and assess the effects of the magnitude, timing, orientation, phase, and spectral content of the energy that is applied to stimulate the cells or tissue. Methods used to model the cell are shown for example in “Spiking Neuron Models: Single Neurons, Populations, Plasticity” by Wulfram Gerstner and Werner M. Kistler (2002); “An Introduction to the Mathematics of Neurons: Modeling in the Frequency Domain (Cambridge Studies in Mathematical Biology)” by F. C. Hoppensteadt (1997); (McNeal, IEEE Trans Biomed Eng 23(4):329-337, 1976); and (Rattay, IEEE Transactions on Biomedical Engineering 36:974-977, 1989).

Next, one examines the effects of stimulation on the functional network (box 6) that is being stimulated or affected, as guided by the integration the effects of stimulation on the targeted cells (as for example could be examined in time, location, cell type, direction of effect (i.e., excite or inhibit cells)). This can be modeled with neural network methods such as those from described in “Spiking Neuron Models: Single Neurons, Populations, Plasticity” by Wulfram Gerstner and Werner M. Kistler (Paperback—Aug. 26, 2002); “An Introduction to the Mathematics of Neurons: Modeling in the Frequency Domain (Cambridge Studies in Mathematical Biology)” by F. C. Hoppensteadt (Paperback—Jun. 28, 1997); Neural Networks: Computational Models and Applications (Studies in Computational Intelligence) by Huajin Tang, Kay Chen Tan and Zhang Yi (Paperback—Nov. 23, 2010); Probabilistic Models of the Brain: Perception and Neural Function (Neural Information Processing) by Rajesh P. N. Rao, Bruno A. Olshausen and Michael S. Lewicki (Hardcover—Feb. 15, 2002); Neural Networks and Intellect: Using Model-Based Concepts by Leonid I. Perlovsky (Hardcover—Oct. 19, 2000); or Neural Network Models by Philippe De Wilde (Paperback—Jul. 11, 1997) but adapted to be driven by the cells (box 5) targeted and driven by the filtered electromagnetic energy (or network sites as modeled to be driven by the filtered electromagnetic energy).

Ultimately the network model (box 6) and/or the targeted cell (box 5) can be used to predict, control, optimize, and/or assess the ultimate systemic effect one is expected to generate from stimulation. Furthermore the computational method and analysis can be integrated with feedback methods such as through the integration of an imaging modality, biofeedback, physiological measures, and/or other measures, such as those exemplified in co-owned and co-pending U.S. patent application Ser. No. 13/162,047.

For example, to computationally determine the effects of electromagnetic field frequency filtering via tissue, one can first model the electromagnetic source and the source energy. One can model the electromagnetic source parameters (such as size, orientation, and materials) and convert the time domain input waveforms of the source energy (i.e., the stimulation source waveform energy) into the frequency domain via discrete Fourier transforms in any computing environment. Second, the electromagnetic field responses of the individual frequency components of the stimulation source to the tissue to be stimulated can be analyzed in the sinusoidal steady state in increments, determined dependent on desired solution resolution, with separate sinusoidal steady state (SSS) computational models, such as finite element methods such as with the Ansoft Maxwell package that numerically solves the problem via a modified T-Ω method or frequency domain finite element models, based on the CAD renderings of the tissue(s) to be stimulated, such as could be developed with an MRI of human head for brain stimulation (where individual tissue components of the model are assigned tissue impedance parameters for the individual tissues based on the frequency components to be analyzed (based on the source energy)) and source properties are included relative to the tissue being stimulated (e.g., the source position (relative to tissue to be stimulated,) orientation (relative to tissue to be stimulated), geometry, and materials). Next, the individual SSS solutions can be combined and used to rebuild a solution in the time domain via inverse Fourier methods (e.g., transforming from the frequency back to the time domain), or the filtered field solutions of the electromagnetic energy in the tissue can be kept in the frequency domain if the next step of cell analysis is to be conducted in the frequency domain. One can examine the electromagnetic energy effects on the tissue, such as analyzing electrochemical or physiological processes taking place in the tissue that might affect its function or vitality (such as for example processes like those explained in Analysis of Transport Phenomena (Topics in Chemical Engineering) by William M. Deen (1998) and Fields, Forces, and Flows in Biological Systems by Alan J. Grodzinsky (2011); or analyzing the energy composition as it relates to tissue safety (such as for example correlating different components of the Poynting vector with energy absorption/storage in the tissue relative to the source and source energy characteristics). The filtered electromagnetic energy waveform is then analyzed as integrated with a cell model, such as a ‘conductance based’ neural model, such as through the current density fields or electrical fields that propagate in the tissue and interact with the cell model through the calculated voltage and current densities in a membrane model (such as a membrane circuit model built of ionic half cell potentials, membrane capacitances, membrane resistances, and channel conductances (which could have a voltage and/or current dependence as driven by the electromagnetic energy stimulating the cell). This model is then used to drive a neural network model and predict the systemic effect on the organism that is stimulated. Ultimately the whole process, or individual components of the process can be interconnected through feedback components and/or controllers, whereby one could direct, tune, and/or optimize the source and/or source energy characteristics to any subcomponent of the analysis.

As another example of the application of such computations, one can determine the optimal energy waveform as a function of the tissue filtering and neural response, such as to optimize the waveform that can have maximum neural effect (such as on a particular neuron type) while remaining with tissue safety guidelines in the tissue (this can be done with a computer control system, such as at the site of the source transducer, which analyzes the effects of the applied energies in simulation or with feedback control, to ultimately adjust the source energy characteristics). As another example, one can model the impact of the electromagnetic energy on the targeted cells as a function of the position of the source transducer; this can be controlled in real time based on imaging data, such as EEG data, and the predicted neural effect in the targeted cells (and the network activity).

These dosing/filtering methods can be implemented with a device that controls the source and source energy parameters, such as an electric circuit or computer controller with an electrical output circuit (that can serve as a function generator to drive the electromagnetic source energy) and/or appropriate mechanical transduction and/or electrical transduction components (such as would be necessary to modify source position and/or shape and/or any component placed between the source transducer and the stimulated tissue(s) (such as a focusing element or a interface element)) which is integrated with a computational component (such as an additional computation circuit, chip, or computational device running software and the methods exemplified in this disclosure, that calculates the effects of the tissue filtering on the applied electromagnetic energy, and/or the effects of the filtered electromagnetic energy on the modeled cells and networks to calculate and guide the dosing of stimulation (such as controlling the timing, orientation, frequency, phase, amplitude, and behavior of the electromagnetic stimulation energy)). This device(s) could also be interconnected through a feedback system, comprised of an additional controller (or by modifying the present controller to assess the feedback information for further system control) and an assessment technology including an imaging technology, biofeedback system, physiological measurement system, patient monitoring device, such as those exemplified in co-owned and co-pending U.S. patent application Ser. No. 13/162,047.

Such a system can include multiple interconnected devices or be built as one single device with multiple subcomponents. These devices can be used with current stimulation devices. For example, one can add an analysis and control chip in the source component of a DBS unit which would tune the waveforms for optimal energy use. For example, the stimulation energy waveforms can be altered based on the total energy output of the system during stimulation (e.g., the total output energy of a voltage controlled or current controlled system is impacted by the filtering of the energies by the tissues (e.g., the current output of a voltage controlled system is dependent on the filtering that takes place on the energy). Thus, the total output energy and the voltage or current control signal (which can be monitored by the control system) can be used to determine the tissue filtering (such as to develop an equation that predicts the filtering taking place at the DBS contacts and/or in the surrounding tissue), and this in turn can be used in an analysis (performed by the analysis and control chip) to optimize the output energy from the system, such as to extend the battery life of the unit).

Turning now to another exemplary embodiment of the invention, one can follow the same procedure outlined in FIG. 1, but focus on the mechanical stimulation dosing/filtering process and method, and focus on the mechanical filtering properties of the tissue. During stimulation, a mechanical energy source (box 1), such as an ultrasound, applies a sonic energy pulse(s) or continuous wave of sonic energy (box 2) to tissue to be stimulated. This can act as a filter to the energy (box 3), resulting in a filtered energy pulse or continuous wave of energy (box 4) in the tissue to be stimulated. The filtered sonic energy stimulates a cell (box 5) in the tissue, such as a neuron or mechanoreceptor, and ultimately affect a functional network of cells (box 6) and leads to systemic effects in the organism (box 7), such as in output behavior of the system being stimulated. This process can be controlled and/or monitored via a feedback mechanism (box 8), active or passive, which modifies any of the elements of the dosing procedure.

When implementing the computational methods, the same types of methods outlined above can be implemented but adjusted for the acoustic field calculations (e.g., the acoustic wave equations analyzed are based on solving for the sonic field solutions and not electrical and magnetic waves, yet the same type of computational methods can be applied as detailed in the examples above). Cell models can also take the form of those discussed above, but adjusted to mechanical interactions and driving effects (such as focusing of mechanical effects via transduction, perturbation, or electromechanical interactions; or developing electromechanical models (or electro-chemical-mechanical models), such as for instance one could model the effects of mechanically moving charged tissue, or altering the impedance of tissue in the presence of charged tissue to generate local electromagnetic field effects).

The methods exemplified herein may be used with multiple energy types. The energies may be applied separately but in a manner whereby the effects of one can precondition the tissue and/or cells to the application of another. The energies can be applied at the same time (with varied or similar patterns), and/or in any combination. Multiple energies may be provided at the same time: whereby energy(ies) may be applied to boost, control, optimize, or tune the effects of other energy(ies); whereby their coupled fields have an effect on the cells, tissue, system, and/or organism; and/or whereby the individual energies operate independently of each other yet have combined effect on the cells, tissue, system, and/or organism. The dosing/filtering methods, in whole or part, may be used to control, optimize, tune, and/or assess the relative: timing, frequency content, amplitude, phase, direction, and/or behavior patterns between the differing energy types and their effects on the cells, tissues, networks, and organisms targeted by the energies. The dosing/filtering methods, could also be used on just one energy type, independent of the other(s).

The methods exemplified herein can be used to control, optimize, assess, direct, or tune the individualized energies or the combined energies with the integrated process (from the source to source energy to filtering network to cell to functional network to systemic effect to the feedback control) between methods, or with individual subcomponents of the process, in any permutation. This dosing/filtering method with multiple energies can be implemented during stimulation, after stimulation, or before stimulation (such as where dosing and filtering analysis could take place via simulation) and in such a way where different energies may be analyzed at the same time and/or at different times in the stimulation process and/or dosing/filtering process. Furthermore, the control, analysis, tuning, and/or optimization of systems with multiple energy types may be connected at any level, in between any parts of the system (or sub groups of multiple energy types), even across dissimilar groups.

Furthermore, multiple effects can be analyzed in any combination; such as for example with multiple cellular effects of stimulation, one for example could analyze the effects of one independent energy on a cellular function and the effects of the combined energy on a second cellular function. The cell models can be used to capture energy effects on the cells response to another energy type(s), and/or the cell can be modeled where it responds in a different physical manner than in the type of energy that is applied (e.g., for a electromechanical stimulation the cell can be modeled to respond in a electromagnetic, mechanical, chemical, optical, and/or thermal manner). These ideas can be applied to cell(s), network(s), organ(s), and/or systemic effect model(s).

When performing computation on multiple energy filtering/dosing, multiple energies may be analyzed, controlled, tuned, and/or optimized: separately (and independently) and/or examined in combined form everywhere and/or at all times and/or at just a location and/or time of interest (such as for example analyzing the energies independently everywhere and at all times, or by analyzing the energies independently everywhere and at all times except at the target location of stimulation and at the time when the individual applied energies are in phase)).

Combined fields can be assessed through methods ranging from a coupled physical analysis to assessing the fields as simply additive in their combined regions. Examples of how energies are combined in tissues and methods of analysis can be found in Continuum Electromechanics by James R. Melcher (1981); Electromechanical Dynamics, Parts 1-3 by Herbert H. Woodson and James R. Melcher (1985); and Fields, Forces, and Flows in Biological Systems by Alan J. Grodzinsky (2011); Analysis of Transport Phenomena (Topics in Chemical Engineering) by William M. Deen (1998); Transport Phenomena, Revised 2nd Edition by R. Byron Bird, Warren E. Stewart and Edwin N. Lightfoot (2006); and Transport Phenomena and Living Systems Biomedical Aspects of Momentum and Mass Transport by Edwin N. Lightfoot (1974).

These combined fields are filtered together, such as one could assess with a tissue electromechanical filtering properties for a electromechanical field. One could for instance analyze one of the energy type's impact on the impedance of a the tissues to the other energy that is applied (and vice versa), such that part of the other energy is affected in some way within the tissues to be stimulated such that now the coupled energies are different in nature than they were before their combination.

The same types of computational methods outlined above can be implemented but adjusted for the combined field calculations (i.e., the computational methods for analyzing sources, energy fields, cell function, filtering, filtered energy fields, functional networks, and systemic effects as outlined above can be implemented, where for example when discussing the analysis of multiple energy fields one could use methods such as computational or analytical methods, separation of variable methods, series expansion methods, finite element methods, variational methods, finite difference methods (e.g., in time domain, frequency domain, spatial domain, etc), moment methods, matrix methods, Monte Carlo methods, perturbation methods, genetic algorithm based methods, iterative methods, and/or optimization methods written in code with languages such as C, C++, Matlab, Mathematica, Fortran, C Sharp, Basic, Java, and/or other programming languages and/or with the use of commercial modeling packages).

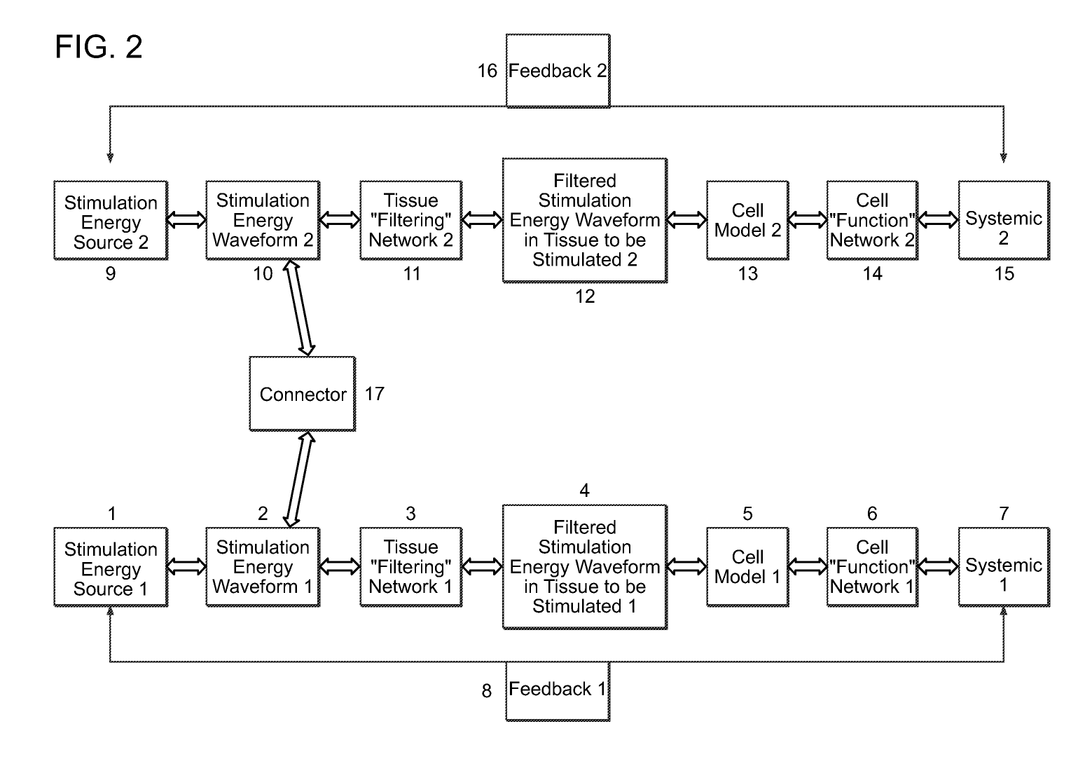

For example, components of the exemplified method may be used to control the timing and/or amplitude of the energies at the source transducers, such as demonstrated in FIG. 2, where two separate energy dosing systems are connected between the source energy waveforms (for example this can be done for optimal energy coupling at the sources with an analysis and control circuit that controls separate transducers (or a single multi-energy transducer) to direct the multiple energy waveforms in magnitude, direction, timing, frequency, and or phase of the energies). In FIG. 2, (box 1) and (box 9) refer to two different energy sources producing two different energy types, (box 2) and (box 10) refer to the stimulation energy waveforms of the two different energy types, (box 3) and (box 11) refer to tissue filtering networks for the individual energy types, (box 4) and (box 12) refer to the filtered energy waveforms in the tissue, (box 5) and (box 13) refer to cell models which represent the cellular response to the individual energy types, (box 6) and (box 14) represent the individual functional network models as influenced by the individual stimulation energies, (box 7) and (box 15) represent the systemic response models, and (box 8) and (box 16) represent feedback between the systems.

In FIG. 2, (box 17) represents a connector that can serve as a control, analysis, and/or communication system between the energy source waveforms, whereby the energy pulse or continuous waveforms can be analyzed in coupled dose or as individualized energies and controlled through this system. This connector (box 17) of the systems could be further integrated through the feedback of the individual systems (box 7) and/or (box 15) (which could also all be integrated as a single controller, analysis, and feedback system for both energies).

This connector between the two energy systems can be implemented at any level, between any individual subparts, of the two energy systems and function as a communication bridge, analysis component, and/or control unit (such as to optimize, tune, or direct energy(ies) in amplitude, timing, frequency, phase, and/or direction), including but not limited to the connecting the analysis or control of any energy system's source transducer, source energy, energy filtering network, cell response models to energy, functional networks response models to energy, and/or systemic effect models with that of another energy system's source transducer, source energy, energy filtering network, cell response models to energy, functional networks response models to energy, and/or systemic effect models (connecting to similar or dissimilar components, with single or multiple connections (such as to connect the source energy waveform controllers of two different systems with the source transducer controller of one of the energy types)). Similarly multiple connectors may be implemented. Furthermore, the connectors can rely on feedback mechanisms (or integrated with the feedback systems of the individual systems), similar to those that have been detailed above (such as in co-owned and co-pending U.S. patent application Ser. No. 13/162,047).

These connectors could also be implemented in a manner just using a subcomponent or subcompenents of the filtering/dosing methods outlined herein. For instance one could develop a connector to control the synchronized application of energies based on the predetermined or modeled characteristics of targeted cells (such as using a neurons characteristics to determine the optimal timing between two energy types). These connectors could also be implemented in a manner independent of filtering/dosing methods outlined herein, but used to control, assess, or bridge the information (between systems and/or subsystems) about the timing, magnitude, frequency, direction, duration, location, and/or phase of energies relative to each other.

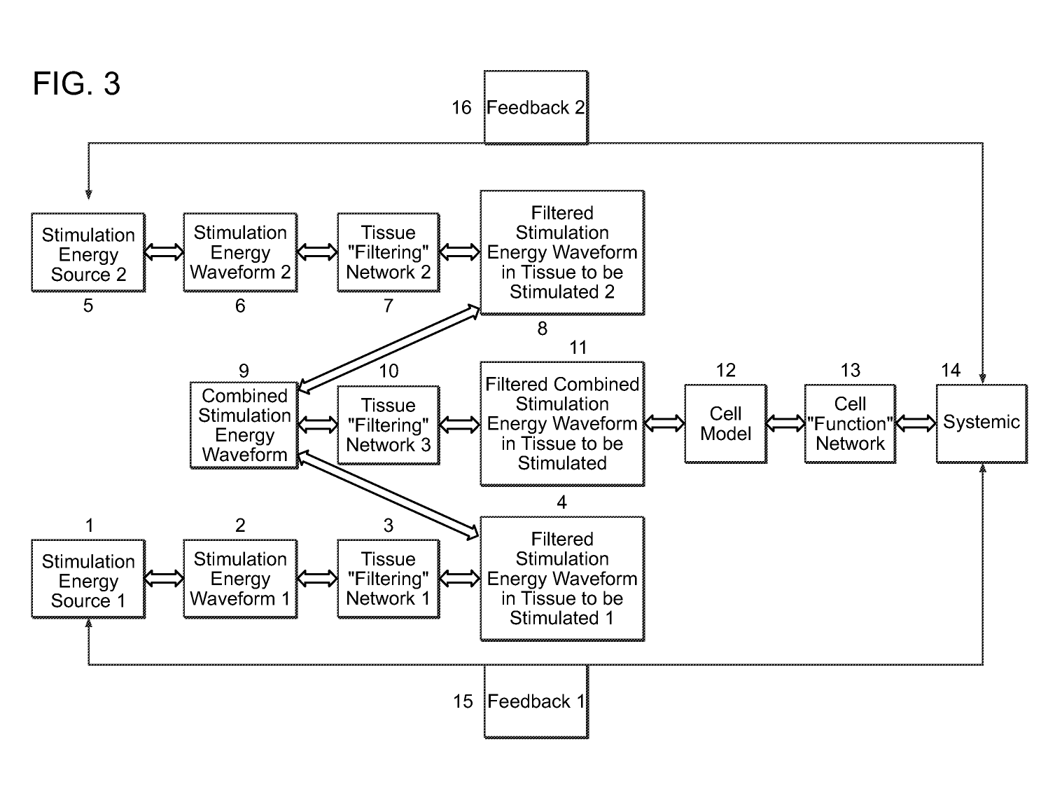

Filtering/dosing analyses on multi-energy source systems can also assess the combined effects of the fields with multiple levels of filtering, such as for example in FIG. 3. In FIG. 3, (box 1) and (box 5) refer to two different energy sources that produce different energy types, (box 2) and (box 6) refer to the stimulation energy waveforms of the two different energy types, (box 3) and (box 7) refer to tissue filtering networks for the individual energy types, (box 4) and (box 8) refer to the filtered energy waveforms in the tissue, (box 9) refers to the combined energies, (box 10) refers to tissue filtering network which impacts the combined energies, (box 11) refers to the filtered combined stimulation energy waveforms, (box 12) represents a cell model of the response to the combined energy, (box 13) the functional network model, and (box 14) a systemic effect model. (Box 15) and (box 16) represent feedbacks between the energy source stimulators and the systemic effect of the system. This dosing/filtering method can be employed to analyze a transcranial electromechanical stimulation procedure, where the brain is being stimulated with an electric field source (such as an electrode) and mechanical field source (such as an ultrasound transducer), which are placed at different locations on the scalp such that the fields are first assessed where the fields are acting independently of each other (e.g., areas of the brain where the two different energy types do not intersect), but then in the locations where the fields are combined (such as in a region of targeted brain tissue) the energies can be analyzed together. As another example, this dosing/filtering method can also be employed to analyze a transcranial electromechanical stimulation where the electric field source and mechanical field source are placed on the same spot on the scalp, but the combined fields are considered negligible (such as they are too low in intensity in a certain tissue, or of negligible importance on the stimulation effects analyzed in a certain tissue or location), but in areas of relevance (such as for location a targeted location in the brain, or locations where the combined fields are high in intensity) the combined energies are analyzed together.

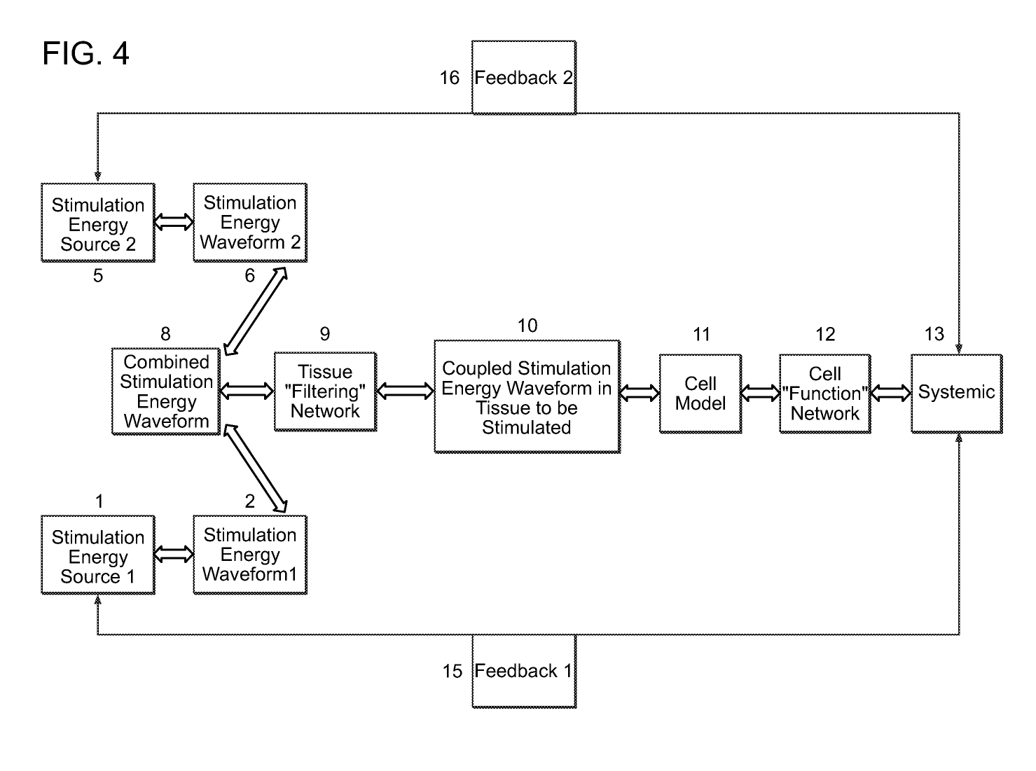

As another example, one can follow the procedure outlined in FIG. 4, which can be employed with a transcranial electromechanical stimulation procedure. During stimulation, a mechanical energy source (box 1) such as an ultrasound applies a sonic energy pulse(s) or continuous wave of sonic energy (box 2) to tissue to be stimulated, and a electromagnetic source (box 5) applies an electromagnetic energy pulse(s) or continuous wave of sonic energy (box 6) to tissue to be stimulated. The energy is applied at the same site and immediately combined (box 8) in the tissue. The combined energy pulse or continuous wave of electromechanical energy is in turn filtered by the tissue filtering network (box 9), wherein the filtered electromechanical energy stimulates a cell (box 10) in said tissue, such as a neuron, and ultimately affect a functional network of cells (box 11) and systemic effects (box 12). This process can be controlled and/or monitored via a feedback mechanism(s) (box 15) and (box 16).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

Using direct measurements of in vivo field-tissue interactions, data herein demonstrate that in vivo tissue impedance properties differ greatly from those classically used to characterize neurostimulation theory and to guide clinical use. For example, tissues carry electromagnetic stimulation currents through both dipole and ionic mechanisms, contrary to previous neurostimulation theory. Neural tissues form an electromagnetic filtering network of resistors and capacitors (and inductors), capable of carrying significant ohmic and displacement currents in a frequency dependent manner. Stimulatory fields are impacted in shape, magnitude, timing, and orientation. In turn, the predicted neural membrane response to stimulation is equally affected. Clinically, these results are far reaching and may lead to a paradigm shift in neurostimulation.

Data herein demonstrate how one could analyze and assess energy dosing based on tissue analysis (conducted prior to stimulation). Tissue recordings were made to measure properties to be implemented in the modeling process, such as for example tissue impedances as a function of applied energy frequency. These tissue impedances were than incorporated into electromagnetic (and electromechanical) models of the tissue energy effects, which can be derived from MRI's of the organisms to be stimulated. These models were used to predict the energy waveforms that propagate in the targeted tissues, such as during TMS and DBS (and tDCS and electromechanical stimulation (EMS)). Models of the energy propagating in the tissue were integrated with models of cell function, with Hodgkin and Huxley like models to predict spiking activity in the cell. The models could also analyzed be extended to impact models, as demonstrated with the field modeling in whole brain simulations. These methods can be integrated to guide dosing, such as for the stimulation of neurons to predict their membrane activity.

### Example 1

### Electromagnetic Analysis of Tissue Impedance Effects based on Spatial-Spectral Filtering

### Methods

In order to ascertain the effects of in-vivo impedance properties on brain stimulation, we first measured the conductivity, σ, and permittivity, ∈, values of head and brain tissues to applied electromagnetic fields in a frequency range from 10 to 50,000 Hz in anesthetized animals. We then constructed MRI guided FEMs of the electromagnetic fields generated during TMS and DBS based on the individual tissue impedance properties recorded in-vivo and with ex-vivo impedance values. We then evaluated how these tissue properties affect the TMS and DBS stimulatory fields. Finally, we explored the effects of the tissues and resulting field responses on stimulation thresholds and response dynamics of a conductance based model of the human motor neuron.

Methods. Tissue Recordings:
Two adult cats were obtained from licensed cat breeders (Liberty Laboratories, Waverly, N.Y.). Neurosurgical/craniotomy procedures, detailed in (Rushmore, Valero-Cabre, Lomber, Hilgetag and Payne, Functional circuitry underlying visual neglect, Brain, 129, (Pt 7), 1803-21, 2006), were conducted. Anaesthetized (4% isoflurane in 30% oxygen and 70% nitrous oxide) animals' head/brain tissues were exposed with a specialized impedance probe fabricated from a modified micro-forceps.

The tissue impedance probe was produced by modifying a self-closing forceps mechanism (Dumont N5) for use as a controllable, two plate probe. Probe tips were created by cutting the tips off of the stainless steel forceps and coating the inside faces using electron beam evaporation. The tips were coated under high vacuum conditions (5×10−7 torr) with 10 nm Titanium (99.99% Alfa Aesar) as an adhesion layer and then 50 nm of Platinum (99.99% Alfa Aesar). The tips were then re-attached to the closing mechanism using two plastic adapter plates, providing electrical insulation from proximal instruments and tissues. The self-closing handle mechanism was also modified using two fine-threaded screws to allow for precise and repeatable control of the inter-electrode separation distance. Further control was achieved by fixing the impedance probe to a micropositioner (Kopf, Tujunga, Calif.). Overall, tissue volume was maintained constant at 50 μm×200 μm×400 μm (+/−10 μm on the larger dimensions).

The probe was used as a surgical instrument to systematically grasp and isolate the tissues, where they were investigated with an HP4192A impedance analyzer (Hewlett Packard, Palo Alto) to determine the tissue impedances (conductivity and permittivity) of the skin, skull, gray matter, and white matter following methods similar to (Hart, Toll, Berner and Bennett, The low frequency dielectric properties of octopus arm muscle measured in vivo, Phys. Med. Biol., 41, (2043-2052, 1996). Recordings were specifically taken from 10 to 50,000 Hz (spanning the typical brain stimulation power spectrum), sweeping the log scale, with approximately 10 sweeps per tissue (at unique locations) per cat, and averaged (i.e., approximately 1300 in-vivo recording points in the power spectrum per tissue). For each tissue, an additional 3-4 sweeps were made at 5 Hz steps (30,000-40,000 additional points), throughout the procedures, to validate the trends presented herein. During the procedures, the effects of in-vivo tissue injury/death were also explored).

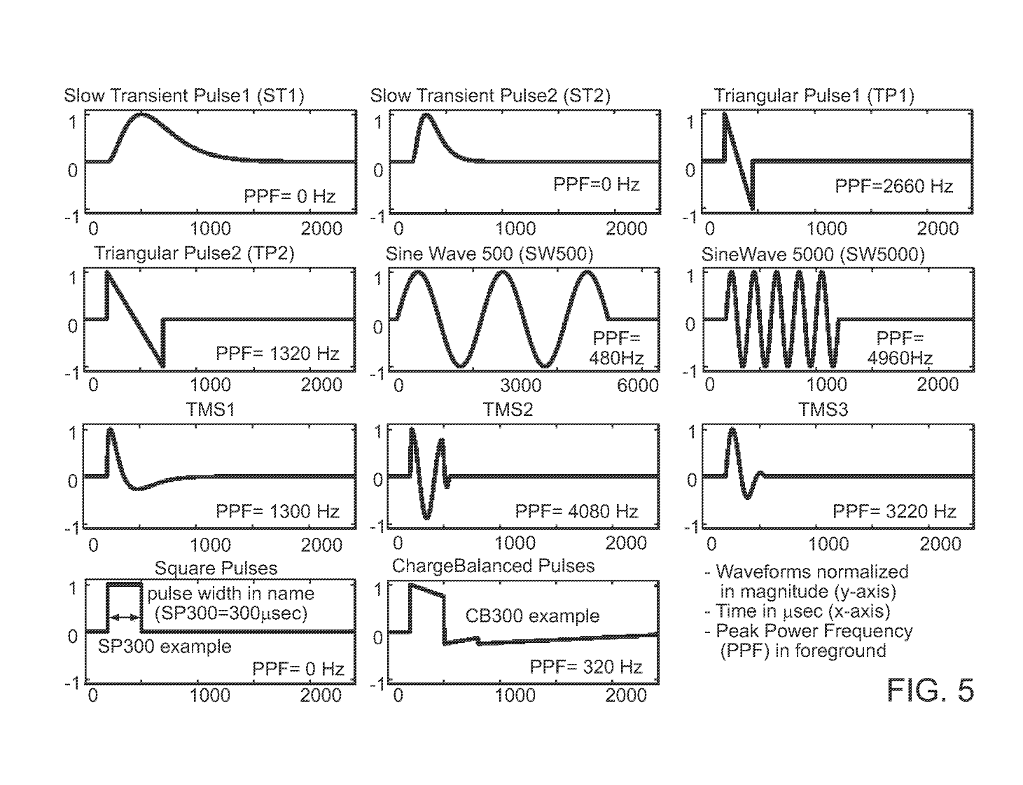

Methods. Transient Electromagnetic Field Solutions of Stimulation:
We constructed MRI guided FEMs of the human head based on the individual tissue impedance properties recorded in-vivo and with ex-vivo impedance values to determine the electromagnetic fields generated during TMS and DBS (the ex-vivo values span the range of those which have served as the basis of neurostimulation theory as demonstrated in (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007)). Nineteen different waveforms commonly used during DBS and TMS stimulation were explored as current constrained TMS coil inputs (3 kA peak), and voltage (0.2 V p-p maximum) and current (0.1 mA p-p maximum) constrained DBS electrode inputs (i.e., we explored the same input waveform shape for the different source conditions), FIG. 5: Waveforms (where For the TMS coil current, DBS constrained voltage, and DBS constrained current, herein normalized to the maximum peak values. Additional square pulses (SP) and charge-balanced pulses (CB) were examined with 65, 600, 1000, and 2000 μs pulse widths (SP's demonstrated 0 Hz peak power frequency components and the CB's 1740, 180, 100, and 40 Hz respectively). Note we evaluated each waveform across all sources (i.e., implementing typical TMS waveforms across DBS sources, and vice versa)).

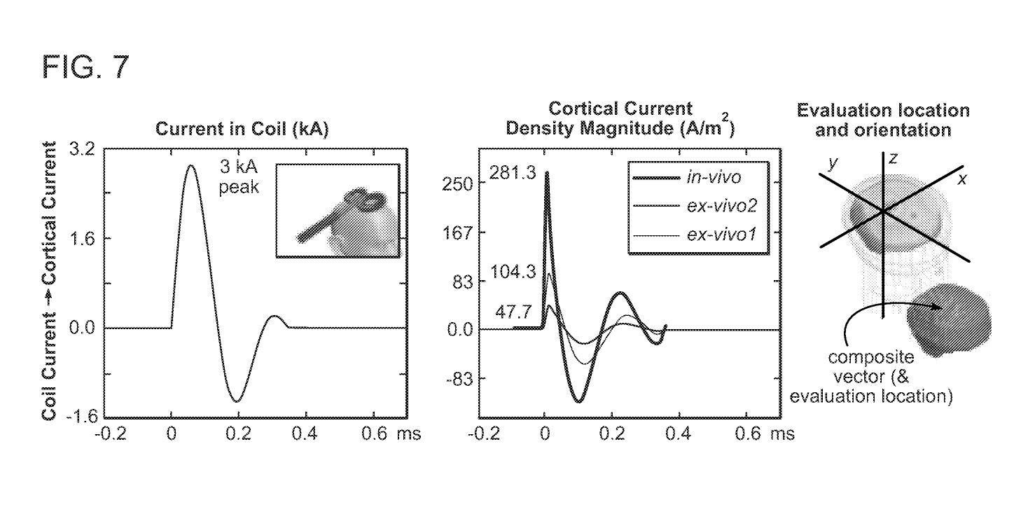

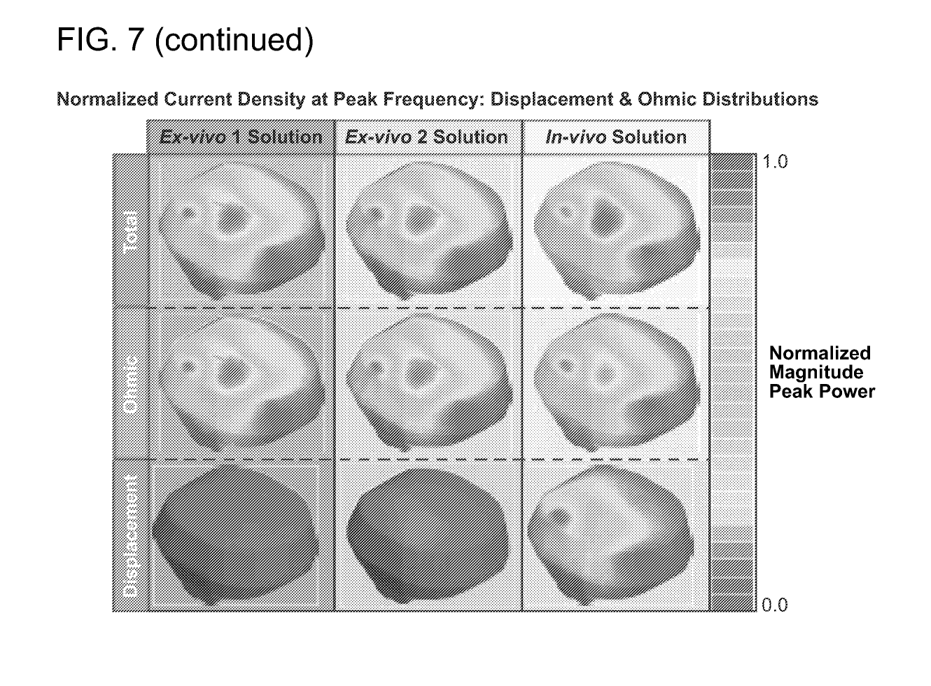

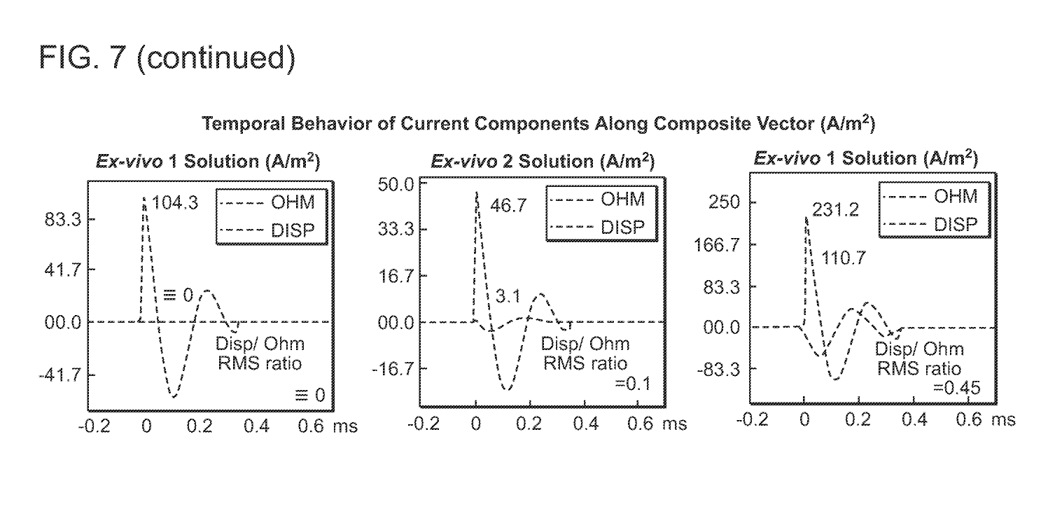

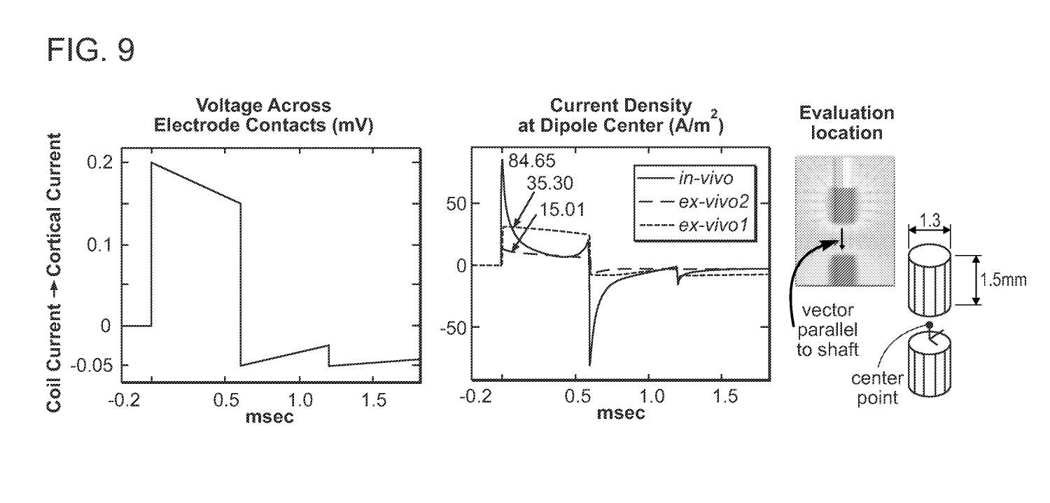

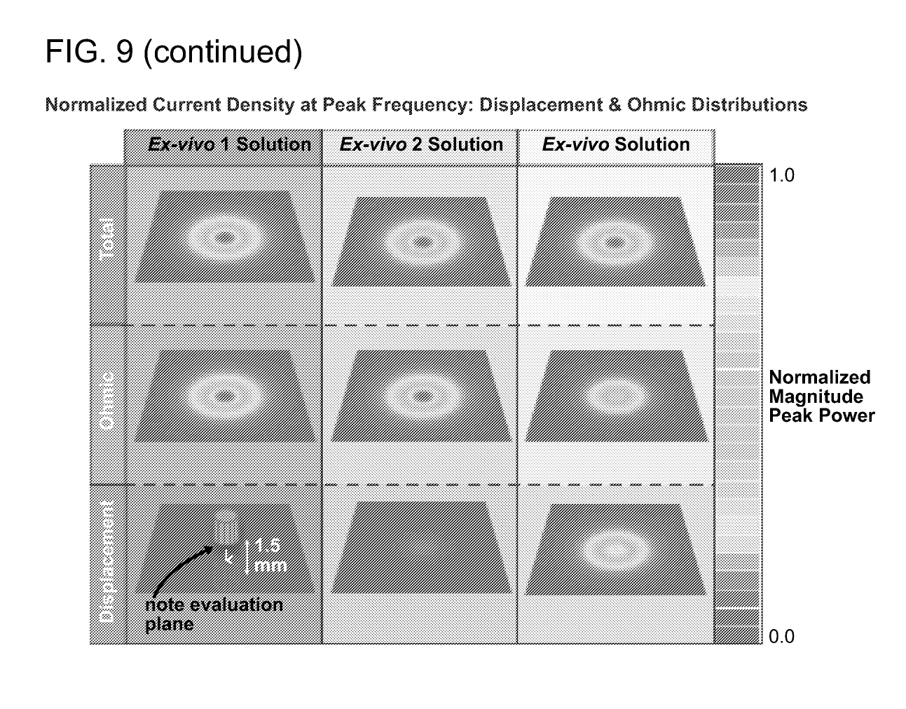

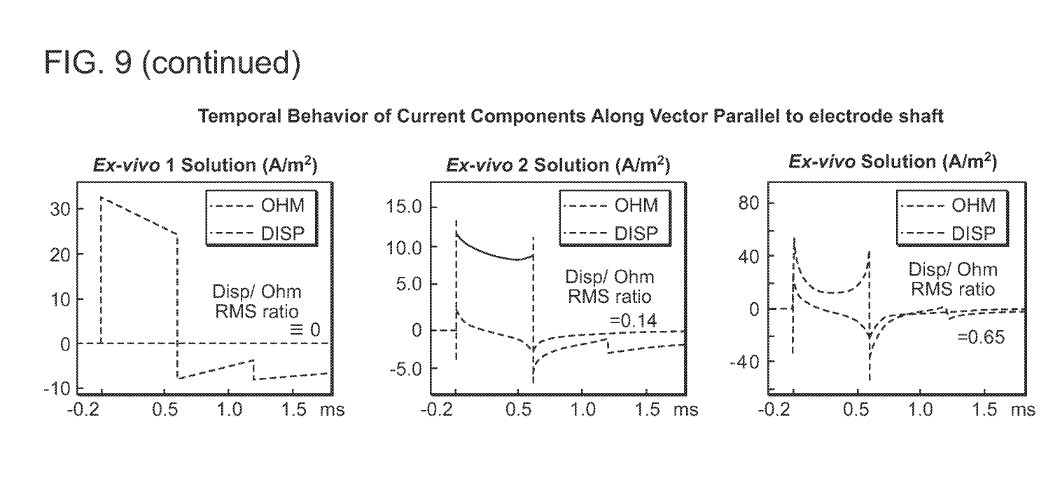

First, the time domain input waveforms were converted to the frequency domain via discrete Fourier transforms in the Mathworks Matlab computing environment. Second, the field responses of the individual frequency components to different tissue impedance sets were analyzed in the sinusoidal steady state in 10 Hz increments with separate TMS and DBS sinusoidal steady state (SSS) FEMs based on MRI guided CAD renderings of the human head explored with a Matlab controlled Ansoft 3D Field Simulator (TMS source: figure-of-eight coil with two 3.5 cm radius windings made of a 25 turn, 7 mm radius copper wire (copper, σ=5.8×10⁷ S/m)/DBS sources: electrode contacts 1.5 mm height/1.3 mm diameters, monopolar and bipolar schemes (dipole 1.5 mm inter-contact distance), contacts (silver, σ=6.7×10⁷ S/m-treated as perfect conductors), lead (plastic σ=6.7×10⁻¹⁵ S/m, ∈ᵣ=3), monopole similar to dipole but with lower contact removed and ground at brain tissue-boundary/see FIGS. 7 and 9 for placement); analysis focused between 0-50 kHz (see (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007) for further details of the SSS computational methodology).

Field solutions were developed for three different tissue impedance sets. The first impedance set used an average of frequency independent conductivity and permittivity magnitudes reflective of ex-vivo values taken from previous brain stimulation studies and most reflective of tissue properties used to develop neurostimulation theory, see for example (Wagner, Zahn, Grodzinsky and Pascual-Leone, Three-dimensional head model simulation of transcranial magnetic stimulation, IEEE Trans Biomed Eng, 51, (9), 1586-98, 2004), (Heller and Hulsteyn, Brain stimulation using electromagnetic sources: theoretical aspects, Biophysical Journal, 63, (129-138, 1992), (Plonsey and Heppner, Considerations of quasi-stationarity in electrophysiological systems, Bull Math Biophys, 29, (4), 657-64, 1967), (Foster and Schwan, Dielectric Properties of Tissues, Biological Effects of Electromagnetic Fields, 25-102, 1996), ((IFAP), Dielectric Properties of body tissues in the frequency range of 10 Hz to 100 GHz—Work reported from the Brooks Air Force Base Report “Compilation of the dielectric properties of body tissues at RF and microwave frequencies” by C. Gabriel., 2007). We refer to the field solutions developed with these values as ‘ex-vivo set 1 solutions.’

The second impedance set used frequency dependent impedance values reported by the Institute of Applied Physics Database ((IFAP), Dielectric Properties of body tissues in the frequency range of 10 Hz to 100 GHz—Work reported from the Brooks Air Force Base Report “Compilation of the dielectric properties of body tissues at RF and microwave frequencies” by C. Gabriel., 2007), which is primarily based on ex-vivo recordings (‘ex-vivo set 2 solutions’).

The final impedance set was based on the recorded tissue permittivity and conductivity values (‘in-vivo solutions’). CSF impedance values reported in the Institute of Applied Physics were used for these derived solutions.

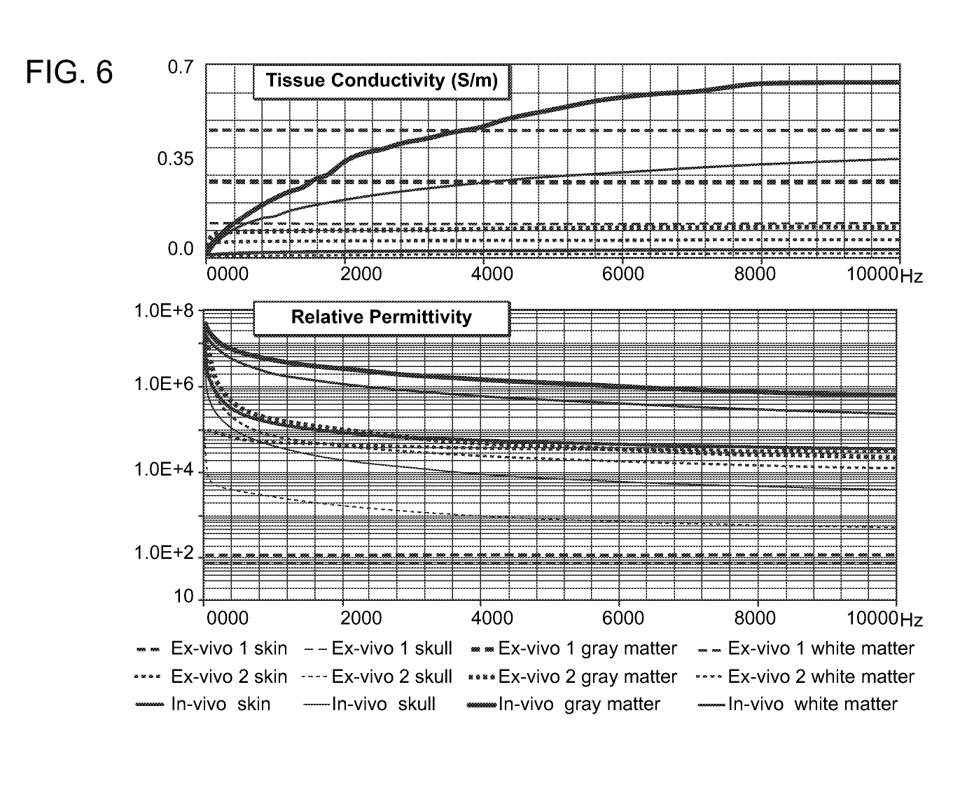

See FIG. 6 for full impedance tabulation from 10-10,000 Hz and Table 1 below for recorded values, and averages used for past brain stimulation studies (Wagner, Zahn, Grodzinsky and Pascual-Leone, Three-dimensional head model simulation of transcranial magnetic stimulation, IEEE Trans Biomed Eng, 51, (9), 1586-98, 2004)) and from the Institute for Applied Physics (IFAP) database ((IFAP/Gabrriel) IFAPD (2007) Dielectric Properties of body tissues in the frequency range of 10 Hz to 100 GHz, reported from the Brooks Air Force Base Report by C. Gabriel (niremf website).

TABLE 1

| | Skin | | Bone | | Gray matter | | White Matter | |
|---|---|---|---|---|---|---|---|---|
| Frequency | Conductance | Relative | Conductance | Relative | Conductance | Relative | Conductance | Relative |
| (Hz) | (S/m) | Permittivity | (S/m) | Permittivity | (S/m) | Permittivity | (S/m) | Permittivity |
| 10 | 9.739E−3 | 1.107E+7 | 1.164E−3 | 2.113E+6 | 1.577E−2 | 3.163E+7 | 1.551E−2 | 3.071E+7 |
| 11.22 | 1.003E−2 | 1.001E+7 | 1.226E−3 | 1.957E+6 | 1.572E−2 | 2.994E+7 | 1.575E−2 | 2.905E+7 |
| 12.589 | 1.033E−2 | 9.176E+6 | 1.230E−3 | 1.738E+6 | 1.606E−2 | 2.845E+7 | 1.597E−2 | 2.722E+7 |
| 14.125 | 1.055E−2 | 8.398E+6 | 1.294E−3 | 1.618E+6 | 1.671E−2 | 2.750E+7 | 1.612E−2 | 2.565E+7 |
| 15.848 | 1.072E−2 | 7.527E+6 | 1.649E−3 | 1.813E+6 | 1.752E−2 | 2.645E+7 | 1.640E−2 | 2.421E+7 |
| 17.782 | 1.105E−2 | 6.896E+6 | 1.725E−3 | 1.657E+6 | 1.789E−2 | 2.512E+7 | 1.663E−2 | 2.250E+7 |
| 19.952 | 1.131E−2 | 6.092E+6 | 1.804E−3 | 1.514E+6 | 1.849E−2 | 2.380E+7 | 1.715E−2 | 2.121E+7 |
| 22.387 | 1.160E−2 | 5.447E+6 | 1.919E−3 | 1.426E+6 | 1.873E−2 | 2.240E+7 | 1.764E−2 | 1.977E+7 |
| 25.118 | 1.198E−2 | 4.873E+6 | 1.962E−3 | 1.273E+6 | 1.951E−2 | 2.129E+7 | 1.827E−2 | 1.856E+7 |
| 28.183 | 1.231E−2 | 4.343E+6 | 2.100E−3 | 1.195E+6 | 2.060E−2 | 2.128E+7 | 1.897E−2 | 1.717E+7 |
| 31.622 | 1.263E−2 | 3.855E+6 | 2.145E−3 | 1.063E+6 | 2.403E−2 | 2.127E+7 | 1.980E−2 | 1.609E+7 |
| 35.481 | 1.291E−2 | 3.423E+6 | 2.302E−3 | 1.000E+6 | 2.802E−2 | 2.150E+7 | 2.046E−2 | 1.495E+7 |
| 39.81 | 1.322E−2 | 3.047E+6 | 2.350E−3 | 8.897E+5 | 3.036E−2 | 2.173E+7 | 2.138E−2 | 1.396E+7 |
| 44.668 | 1.353E−2 | 2.668E+6 | 2.445E−3 | 8.110E+5 | 3.299E−2 | 2.123E+7 | 2.249E−2 | 1.303E+7 |
| 50.118 | 1.389E−2 | 2.377E+6 | 2.546E−3 | 7.400E+5 | 3.694E−2 | 2.093E+7 | 2.358E−2 | 1.216E+7 |
| 56.234 | 1.424E−2 | 2.097E+6 | 2.605E−3 | 6.543E+5 | 3.880E−2 | 1.988E+7 | 2.576E−2 | 1.172E+7 |
| 63.095 | 1.449E−2 | 1.854E+6 | 2.765E−3 | 6.022E+5 | 4.186E−2 | 1.920E+7 | 2.869E−2 | 1.149E+7 |
| 70.794 | 1.474E−2 | 1.633E+6 | 2.841E−3 | 5.394E+5 | 4.542E−2 | 1.849E+7 | 3.244E−2 | 1.135E+7 |
| 79.432 | 1.493E−2 | 1.523E+6 | 2.939E−3 | 4.924E+5 | 4.905E−2 | 1.759E+7 | 3.934E−2 | 1.189E+7 |
| 89.125 | 1.520E−2 | 1.450E+6 | 2.944E−3 | 4.353E+5 | 5.156E−2 | 1.624E+7 | 4.469E−2 | 1.145E+7 |
| 100 | 1.541E−2 | 1.277E+6 | 3.123E−3 | 3.996E+5 | 5.449E−2 | 1.528E+7 | 4.848E−2 | 1.083E+7 |
| 112.201 | 1.574E−2 | 1.122E+6 | 3.208E−3 | 3.544E+5 | 5.872E−2 | 1.465E+7 | 5.193E−2 | 1.013E+7 |
| 125.892 | 1.601E−2 | 9.866E+5 | 3.281E−3 | 3.177E+5 | 6.284E−2 | 1.387E+7 | 5.613E−2 | 9.542E+6 |
| 141.253 | 1.627E−2 | 8.773E+5 | 3.484E−3 | 2.911E+5 | 6.719E−2 | 1.309E+7 | 6.017E−2 | 8.881E+6 |
| 158.489 | 1.665E−2 | 7.740E+5 | 3.562E−3 | 2.599E+5 | 7.150E−2 | 1.226E+7 | 6.535E−2 | 8.231E+6 |
| 177.827 | 1.700E−2 | 6.851E+5 | 3.656E−3 | 2.302E+5 | 7.518E−2 | 1.126E+7 | 6.958E−2 | 7.598E+6 |
| 199.526 | 1.723E−2 | 5.974E+5 | 3.729E−3 | 2.048E+5 | 8.014E−2 | 1.056E+7 | 7.439E−2 | 7.036E+6 |
| 223.872 | 1.741E−2 | 5.310E+5 | 3.962E−3 | 1.888E+5 | 8.544E−2 | 9.720E+6 | 7.895E−2 | 6.484E+6 |
| 251.188 | 1.764E−2 | 4.710E+5 | 4.043E−3 | 1.676E+5 | 9.257E−2 | 9.293E+6 | 8.365E−2 | 5.971E+6 |
| 281.838 | 1.801E−2 | 4.217E+5 | 4.120E−3 | 1.488E+5 | 9.948E−2 | 8.774E+6 | 8.888E−2 | 5.506E+6 |
| 316.227 | 1.826E−2 | 3.737E+5 | 4.203E−3 | 1.310E+5 | 1.073E−1 | 8.316E+6 | 9.397E−2 | 5.057E+6 |
| 354.813 | 1.846E−2 | 3.334E+5 | 4.276E−3 | 1.160E+5 | 1.152E−1 | 7.746E+6 | 9.947E−2 | 4.641E+6 |
| 398.107 | 1.884E−2 | 3.029E+5 | 4.345E−3 | 1.028E+5 | 1.232E−1 | 7.187E+6 | 1.053E−1 | 4.250E+6 |
| 446.683 | 1.906E−2 | 2.705E+5 | 4.618E−3 | 9.494E+4 | 1.338E−1 | 6.743E+6 | 1.109E−1 | 3.885E+6 |
| 501.187 | 1.943E−2 | 2.455E+5 | 4.622E−3 | 8.283E+4 | 1.413E−1 | 6.177E+6 | 1.170E−1 | 3.549E+6 |
| 562.341 | 1.970E−2 | 2.191E+5 | 4.710E−3 | 7.346E+4 | 1.519E−1 | 5.790E+6 | 1.238E−1 | 3.252E+6 |
| 630.957 | 2.000E−2 | 1.980E+5 | 4.799E−3 | 6.522E+4 | 1.626E−1 | 5.394E+6 | 1.295E−1 | 2.947E+6 |
| 707.945 | 2.028E−2 | 1.805E+5 | 4.884E−3 | 5.780E+4 | 1.762E−1 | 5.110E+6 | 1.367E−1 | 2.690E+6 |
| 794.328 | 2.059E−2 | 1.643E+5 | 4.968E−3 | 5.132E+4 | 1.895E−1 | 4.796E+6 | 1.425E−1 | 2.480E+6 |
| 891.25 | 2.087E−2 | 1.485E+5 | 5.027E−3 | 4.531E+4 | 2.030E−1 | 4.408E+6 | 1.490E−1 | 2.194E+6 |
| 1000 | 2.115E−2 | 1.346E+5 | 5.108E−3 | 4.008E+4 | 2.176E−1 | 4.091E+6 | 1.519E−1 | 1.935E+6 |
| 1122.018 | 2.147E−2 | 1.233E+5 | 5.186E−3 | 3.557E+4 | 2.302E−1 | 3.724E+6 | 1.667E−1 | 1.836E+6 |
| 1258.925 | 2.182E−2 | 1.134E+5 | 5.268E−3 | 3.153E+4 | 2.443E−1 | 3.433E+6 | 1.750E−1 | 1.666E+6 |
| 1412.537 | 2.212E−2 | 1.045E+5 | 5.318E−3 | 2.780E+4 | 2.575E−1 | 3.104E+6 | 1.842E−1 | 1.510E+6 |
| 1584.893 | 2.248E−2 | 9.639E+4 | 5.394E−3 | 2.466E+4 | 2.849E−1 | 2.951E+6 | 1.928E−1 | 1.367E+6 |
| 1778.279 | 2.292E−2 | 8.917E+4 | 5.439E−3 | 2.177E+4 | 3.049E−1 | 2.700E+6 | 2.020E−1 | 1.234E+6 |
| 1995.262 | 2.332E−2 | 8.260E+4 | 5.526E−3 | 1.936E+4 | 3.497E−1 | 2.592E+6 | 2.114E−1 | 1.117E+6 |
| 2238.721 | 2.373E−2 | 7.699E+4 | 5.568E−3 | 1.707E+4 | 3.782E−1 | 2.398E+6 | 2.213E−1 | 1.006E+6 |
| 2511.886 | 2.417E−2 | 7.174E+4 | 5.639E−3 | 1.518E+4 | 3.924E−1 | 2.128E+6 | 2.313E−1 | 9.066E+5 |
| 2818.382 | 2.463E−2 | 6.703E+4 | 5.673E−3 | 1.342E+4 | 4.155E−1 | 1.939E+6 | 2.424E−1 | 8.187E+5 |
| 3162.277 | 2.510E−2 | 6.262E+4 | 5.739E−3 | 1.193E+4 | 4.303E−1 | 1.718E+6 | 2.522E−1 | 7.334E+5 |
| 3548.133 | 2.560E−2 | 5.865E+4 | 5.794E−3 | 1.021E+4 | 4.559E−1 | 1.593E+6 | 2.626E−1 | 6.572E+5 |
| 3981.071 | 2.613E−2 | 5.504E+4 | 5.858E−3 | 9.078E+3 | 4.740E−1 | 1.419E+6 | 2.731E−1 | 5.875E+5 |
| 4466.835 | 2.667E−2 | 5.162E+4 | 5.884E−3 | 8.090E+3 | 5.107E−1 | 1.317E+6 | 2.848E−1 | 5.280E+5 |
| 5011.872 | 2.722E−2 | 4.844E+4 | 5.941E−3 | 7.219E+3 | 5.384E−1 | 1.192E+6 | 2.959E−1 | 4.700E+5 |
| 5623.413 | 2.780E−2 | 4.559E+4 | 5.958E−3 | 6.445E+3 | 5.692E−1 | 1.064E+6 | 3.051E−1 | 4.182E+5 |
| 6309.573 | 2.839E−2 | 4.286E+4 | 5.994E−3 | 5.767E+3 | 5.919E−1 | 9.441E+5 | 3.162E−1 | 3.726E+5 |
| 7079.457 | 2.900E−2 | 4.034E+4 | 6.028E−3 | 5.189E+3 | 6.029E−1 | 8.197E+5 | 3.274E−1 | 3.312E+5 |
| 7943.282 | 2.972E−2 | 3.799E+4 | 6.047E−3 | 4.680E+3 | 6.302E−1 | 7.321E+5 | 3.391E−1 | 2.928E+5 |
| 8912.509 | 3.040E−2 | 3.582E+4 | 6.084E−3 | 4.223E+3 | 6.351E−1 | 6.614E+5 | 3.495E−1 | 2.591E+5 |
| 10000 | 3.111E−2 | 3.376E+4 | 6.102E−3 | 3.914E+3 | 6.360E−1 | 6.071E+5 | 3.595E−1 | 2.286E+5 |
| 11220.18 | 3.184E−2 | 3.180E+4 | 6.154E−3 | 3.499E+3 | 6.368E−1 | 4.649E+5 | 3.711E−1 | 2.026E+5 |
| 12589.25 | 3.969E−2 | 3.650E+4 | 6.192E−3 | 3.207E+3 | 6.527E−1 | 4.054E+5 | 3.836E−1 | 1.797E+5 |
| 14125.37 | 4.045E−2 | 3.419E+4 | 6.224E−3 | 2.946E+3 | 6.716E−1 | 3.558E+5 | 3.926E−1 | 1.575E+5 |
| 15848.93 | 4.117E−2 | 3.202E+4 | 6.269E−3 | 2.719E+3 | 6.914E−1 | 3.121E+5 | 4.030E−1 | 1.391E+5 |
| 17782.79 | 4.201E−2 | 3.003E+4 | 6.350E−3 | 2.445E+3 | 6.927E−1 | 2.634E+5 | 4.133E−1 | 1.229E+5 |
| 19952.62 | 4.291E−2 | 2.820E+4 | 6.373E−3 | 2.357E+3 | 7.145E−1 | 2.328E+5 | 4.244E−1 | 1.088E+5 |
| 22387.21 | 4.391E−2 | 2.649E+4 | 6.426E−3 | 2.219E+3 | 7.172E−1 | 1.978E+5 | 4.335E−1 | 9.504E+4 |
| 25118.86 | 4.486E−2 | 2.486E+4 | 6.507E−3 | 2.091E+3 | 7.474E−1 | 1.767E+5 | 4.464E−1 | 8.404E+4 |
| 28183.82 | 4.582E−2 | 2.339E+4 | 6.600E−3 | 1.988E+3 | 7.639E−1 | 1.521E+5 | 4.557E−1 | 7.353E+4 |
| 31622.77 | 4.687E−2 | 2.200E+4 | 6.700E−3 | 1.894E+3 | 7.983E−1 | 1.369E+5 | 4.658E−1 | 6.445E+4 |
| 35481.33 | 4.801E−2 | 2.071E+4 | 6.807E−3 | 1.813E+3 | 8.159E−1 | 1.177E+5 | 4.751E−1 | 5.609E+4 |
| 39810.71 | 4.925E−2 | 1.952E+4 | 6.943E−3 | 1.746E+3 | 8.398E−1 | 1.048E+5 | 4.833E−1 | 4.907E+4 |
| 44668.35 | 5.071E−2 | 1.842E+4 | 7.096E−3 | 1.687E+3 | 8.444E−1 | 8.876E+4 | 4.913E−1 | 4.273E+4 |
| 50118.72 | 5.217E−2 | 1.738E+4 | 7.274E−3 | 1.639E+3 | 8.486E−1 | 7.749E+4 | 5.015E−1 | 3.719E+4 |

Finally, time domain solutions were rebuilt with inverse Fourier transforms of the SSS field solutions. The transient electrical field and current density waveforms were then analyzed in terms of field magnitudes, orientations, focality (i.e., area/volume of stimulated region), and penetration in a manner explained in (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007), as a function of time and tissue impedance. The evaluation point for TMS metrics reported (e.g. current density magnitude, electric field magnitude, etc) is illustrated in the top right corner of FIG. 7, and for DBS in FIG. 9.

Methods. Conductance Based Neural Modeling:
Conductance-based compartmental models of brain stimulation were generated based on the McNeal Model (McNeal, Analysis of a model for excitation of myelinated nerve, IEEE Trans Biomed Eng, 23, (4), 329-37, 1976), as optimized by Rattay (Rattay, Analysis of models for extracellular fiber stimulation, IEEE Transactions on Biomedical Engineering, 36, (974-977, 1989), with the external driving field determined as above. Human motor neuron parameters were drawn from (Traub, Motorneurons of different geometry and the size principle, Biol Cybern, 25, (3), 163-76, 1977), (Jones and Bawa, Computer simulation of the responses of human motoneurons to composite 1A EPSPS: effects of background firing rate, J Neurophysiol, 77, (1), 405-20, 1997), and the initial segment served as the focus of our calculations (Table 2: Human Motor Neuron Membrane Properties provided below). By analyzing the tissue as we did and developing the field solutions with filtering considered these methods allow for solutions not attainable with these classic models.

TABLE 2
Human Motor Neuron Membrane Properties: Initial segment properties
and equations-for further details see (Traub, Motorneurons of different
geometry and the size principle, Biol Cybern, 25, (3), 163-76, 1977),
(Jones and Bawa, Computer simulation of the responses of human
motoneurons to composite 1A EPSPS: effects of background firing rate,
J Neurophysiol, 77, (1), 405-20, 1997).

| | | |
|---|---|---|
| Initial segment length | 100 | micron |
| Initial segment compartment length | 20 | micron |
| Initial segment diameter | 5 | micron |
| Capacitance of membrane | 1 | microFarad/cm² |
| Axonal resistivity | 70 | ohm cm |
| g_{Na} | 500 | mS/cm² |
| E_{Na} | 115 | mV |
| g_{K} | 100 | mS/cm² |
| E_{K} | −10 | mV |

| | |
|---|---|
| I_{Na} | g_{Na}*m³*h*(Vₘ − E_{Na}) |
| I_{K} | g_{K}*n⁴*(Vₘ − E_{Na}) |
| αₘ | (4 − 0.4* Vₘ)/(e⁽⁽¹* ^{Vm-10)/-5)} − 1 |
| αₕ | 0.16/e^{((Vm-37.78)/-18.14)} |
| αₙ | (0.2 − 0.02*Vₘ)/(e^{((Vm-10)/-10)} − 1) |
| βₘ | (0.4* Vₘ − 14)/((e⁽¹*^{Vm-35)/5}) − 1) |
| βₕ | 4/(e^{(3-0.1}*^{Vm)} + 1) |
| βₙ | 0.15/(e^{((Vm-33.79/71.86)} − 0.01 |

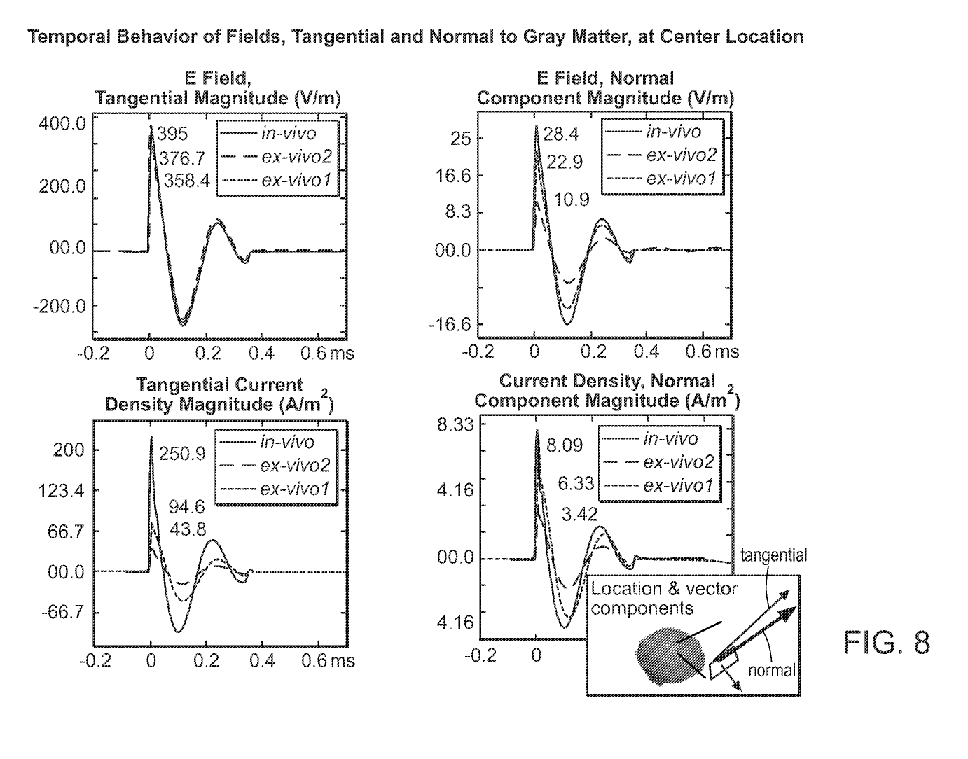

Membrane dynamics were solved using Euler's method at a time interval of 10⁻⁶ sec. Neurostimulation thresholds were calculated by integrating the field solution with these compartmental models. For each stimulating waveform, source, and tissue property model, we performed an iterative search to find the smallest constrained input (TMS constrained coil currents, DBS constrained electrode currents, and DBS constrained electrode voltages) that generated an action potential, all reported in terms of peak waveform values of the constrained input. For TMS coil current inputs, we report the thresholds for neurons oriented approximately parallel to the figure-of-eight coil intersection (along the composite vector in FIG. 7) and oriented approximately normal to the gray matter-CSF tissue-boundary (FIG. 8). For both the dipole and monopole DBS constrained current inputs, we report the thresholds for neurons oriented parallel to the electrode shaft. Although it was expected that the thresholds would be the same for the varied impedance sets for the voltage constrained DBS models (as we used a variation of the McNeal model based on a voltage based activation function), they were also calculated as a redundancy check of the integrated field solver and neuromembrane methods. For all conditions analyzed, transmembrane ionic flow (sodium and potassium) was also monitored.

Methods. Statistical Analysis:
We compared the electromagnetic field properties and neural thresholds in tissues with ex-vivo and in-vivo impedance properties for TMS and DBS stimulation sources. For each stimulation field, we compared RMS current densities, peak current densities, RMS electric field magnitudes, peak electric field magnitudes, and the RMS displacement to ohmic current density ratios. We also compared neural thresholds computed for conductance-based neural models of the human motor neuron. For each comparison, statistical significance was determined by Wilcoxon signed-rank tests at a significance level of p<0.01.

### Results:

Results. Tissue Recordings:
We first measured the conductivity and permittivity values of head tissues to applied electromagnetic fields in a frequency range from 10 to 50,000 Hz in-vivo. The results of these measurements are shown in FIG. 6 as a function of stimulation frequency. The recorded tissue values of the skin, skull, gray matter, and white matter differed in magnitude and degree of frequency response from previous ex-vivo values reported in the literature that guide neurostimulation theory. Recorded conductivity values were on the order of magnitudes reported from past studies, but demonstrated a more sizable frequency response for all of the tissues, and a slightly increased conductivity for the brain tissues than most earlier reports (FIG. 6, top row). However, the largest differences between the tissue recordings and those reported in the literature were seen in the tissues' relative permittivity magnitudes at low frequencies (FIG. 6, bottom row). For example in FIG. 6, at a 5 kHz center point of the typical TMS frequency band (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007), the recorded relative permittivity magnitudes for the gray matter (solid black line) was approximately two orders of magnitude higher than those reported in primarily excised tissues of the Institute of Applied Physics Database ((IFAP), Dielectric Properties of body tissues in the frequency range of 10 Hz to 100 GHz—Work reported from the Brooks Air Force Base Report “Compilation of the dielectric properties of body tissues at RF and microwave frequencies” by C. Gabriel., 2007) (dotted black line) and over five orders of magnitude higher than values most commonly used in past brain stimulation studies (dashed black line)(Wagner, Zahn, Grodzinsky and Pascual-Leone, Three-dimensional head model simulation of transcranial magnetic stimulation, IEEE Trans Biomed Eng, 51, (9), 1586-98, 2004).

Importantly, the range of permittivity values that we recorded was consistent with measures from studies in which values were recorded under in-vivo conditions in other non-brain tissue types (Hart, Toll, Berner and Bennett, The low frequency dielectric properties of octopus arm muscle measured in vivo, Phys. Med. Biol., 41, (2043-2052, 1996), ((IFAP), Dielectric Properties of body tissues in the frequency range of 10 Hz to 100 GHz—Work reported from the Brooks Air Force Base Report “Compilation of the dielectric properties of body tissues at RF and microwave frequencies” by C. Gabriel., 2007), (Yamamoto and Yamamoto, Electrical properties of the epidermal stratum corneum, Med Biol Eng, 14, (2), 151-8, 1976). We also found that permittivity and conductivity decreased in magnitude with time post tissue injury/death, approaching ex-vivo values reported in the literature ((IFAP), Dielectric Properties of body tissues in the frequency range of 10 Hz to 100 GHz—Work reported from the Brooks Air Force Base Report “Compilation of the dielectric properties of body tissues at RF and microwave frequencies” by C. Gabriel., 2007).

Results. Tissue Effects on the TMS Fields:
We constructed MRI guided finite element models (FEM) of human head based on the individual tissue impedance properties, recorded in-vivo and with ex-vivo values, to calculate the electromagnetic fields generated during TMS (the ex-vivo values span the range of those which have served as the basis of neurostimulation theory (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007)). We then compared the TMS induced, time dependent, field distributions, as a function of these different tissue impedance sets. Spatial and temporal snapshots of the resulting current densities for 1 stimulation waveform (TMS 3, triphasic wave) are shown in FIGS. 7&8. The top panel of FIG. 7 shows the stimulation current input in the TMS coil on the left and the resulting current waveforms directly under the coil in the cortex for the in-vivo and ex-vivo impedance values. The magnitude of the current density from the in-vivo measurements is notably higher than that of either of the ex-vivo solutions. As a function of tissue impedance, the electric fields showed similar altered behavior, but with significant decreases in the distributions' in-vivo magnitude (FIGS. 7 and 8, & Table 3 below).

TABLE 3
TMS Field Solutions for a Figure-of-Eight Coil Source (evaluated along the composite field vector, centered to coil
intersection 2.3 cm from coil face in the Gray Matter (Input: 3 kA Peak Current in 25 Turn Air Core Copper Coil))

| | B. Displacement | | | | | | | | | | | | | | |
|---|---|---|---|---|---|---|---|---|---|---|---|---|---|---|---|
| | to Ohmic RMS | | | C. RMS Current | | | D. Peak Current | | | E. RMS Electric | | | F. Peak Electric Field | | |
| | Current Ratio | | | Density (A/m\hat{ }2) | | | Density (A/m\hat{ }2) | | | Field (V/m) | | | (V/m) | | |
| A. Coil | Ex- | Ex- | | Ex- | Ex- | | Ex- | Ex- | | Ex- | Ex- | | Ex- | Ex- | |
| Input | vivo | vivo | In- | vivo | vivo | In- | vivo | vivo | In- | vivo | vivo | In- | vivo | vivo | In- |
| Waveform | 1 | 2 | vivo | 1 | 2 | vivo | 1 | 2 | vivo | 1 | 2 | vivo | 1 | 2 | vivo |
| SP 65 μs | 0.00065 | 0.07 | 0.49 | 81.25 | 38.40 | 228.42 | 443.91 | 211.02 | 1163.49 | 294.25 | 304.83 | 244.33 | 1607.55 | 1660.85 | 1525.85 |
| CBP 65 | 0.00065 | 0.07 | 0.48 | 182.81 | 86.01 | 499.36 | 438.87 | 208.52 | 1147.08 | 662.00 | 685.78 | 551.06 | 1589.45 | 1640.69 | 1503.40 |
| μs | | | | | | | | | | | | | | | |
| SP 300 μs | 0.00065 | 0.07 | 0.50 | 85.85 | 40.39 | 239.37 | 443.94 | 210.92 | 1153.07 | 310.81 | 321.50 | 258.79 | 1607.21 | 1660.03 | 1528.26 |
| CBP 300 | 0.00065 | 0.07 | 0.50 | 85.84 | 40.39 | 239.42 | 442.83 | 210.37 | 1150.14 | 310.77 | 321.47 | 258.86 | 1603.36 | 1655.14 | 1523.39 |
| μs | | | | | | | | | | | | | | | |
| SP 600 μs | 0.00065 | 0.07 | 0.50 | 60.75 | 28.58 | 169.51 | 443.94 | 210.92 | 1153.07 | 219.93 | 227.50 | 183.12 | 1607.21 | 1660.03 | 1528.26 |
| CBP 600 | 0.00065 | 0.07 | 0.50 | 60.76 | 28.59 | 169.61 | 443.48 | 210.64 | 1151.98 | 219.96 | 227.65 | 183.27 | 1605.53 | 1657.65 | 1526.53 |
| μs | | | | | | | | | | | | | | | |
| SP 1000 | 0.00065 | 0.07 | 0.50 | 47.09 | 22.15 | 131.44 | 444.08 | 211.00 | 1153.55 | 170.43 | 176.44 | 141.98 | 1607.42 | 1661.60 | 1528.86 |
| μs | | | | | | | | | | | | | | | |
| CBP 1000 | 0.00065 | 0.07 | 0.50 | 47.09 | 22.15 | 131.45 | 443.73 | 210.72 | 1152.66 | 170.42 | 176.46 | 141.99 | 1606.28 | 1658.18 | 1527.39 |
| μs | | | | | | | | | | | | | | | |
| SP 2000 | 0.00065 | 0.07 | 0.50 | 33.30 | 15.67 | 92.97 | 444.10 | 211.11 | 1153.72 | 120.53 | 125.00 | 100.43 | 1607.33 | 1663.82 | 1528.90 |
| μs | | | | | | | | | | | | | | | |
| CBP 2000 | 0.00065 | 0.07 | 0.50 | 33.30 | 15.68 | 92.97 | 443.90 | 210.86 | 1153.26 | 120.53 | 125.05 | 100.43 | 1606.76 | 1660.49 | 1528.17 |
| μs | | | | | | | | | | | | | | | |
| ST1 | 0.00004 | 0.14 | 0.57 | 6.79 | 2.49 | 7.64 | 19.77 | 7.45 | 26.27 | 24.30 | 25.32 | 24.08 | 70.96 | 73.92 | 69.75 |
| ST2 | 0.00009 | 0.12 | 0.47 | 15.35 | 6.12 | 26.13 | 47.53 | 19.57 | 90.21 | 55.33 | 57.43 | 53.84 | 171.55 | 179.03 | 166.11 |
| SW500 | 0.00003 | 0.17 | 0.78 | 7.16 | 2.47 | 5.31 | 11.73 | 5.30 | 27.59 | 25.45 | 26.41 | 25.56 | 42.07 | 44.96 | 40.23 |
| SW5000 | 0.00011 | 0.10 | 0.43 | 76.46 | 33.27 | 167.29 | 112.68 | 52.33 | 308.89 | 276.51 | 295.41 | 266.05 | 407.78 | 433.02 | 392.35 |
| TP 1 | 0.00065 | 0.07 | 0.51 | 57.60 | 27.14 | 161.58 | 433.42 | 208.12 | 1173.05 | 208.58 | 215.45 | 174.69 | 1569.95 | 1614.94 | 1487.17 |
| TP 2 | 0.00065 | 0.07 | 0.50 | 61.63 | 29.02 | 172.41 | 438.81 | 210.10 | 1167.25 | 223.13 | 230.77 | 186.31 | 1589.33 | 1636.94 | 1507.41 |
| TMS 1 | 0.00047 | 0.08 | 0.48 | 67.26 | 30.61 | 174.79 | 291.41 | 137.33 | 781.76 | 243.37 | 253.81 | 219.11 | 1055.09 | 1095.65 | 1002.44 |
| TMS 2 | 0.00041 | 0.09 | 0.50 | 70.52 | 31.81 | 179.95 | 346.00 | 161.43 | 936.78 | 255.16 | 267.11 | 242.21 | 1253.08 | 1298.39 | 1190.93 |
| TMS 3 | 0.00017 | 0.10 | 0.45 | 35.32 | 14.99 | 74.95 | 104.32 | 47.70 | 281.27 | 127.65 | 133.75 | 122.34 | 377.40 | 396.05 | 358.61 |

TMS Field Solutions for a Figure-of-Eight Coil Source (evaluated along the normal-field vector, centered to coil
intersection 2.3 cm from coil face in the Gray Matter (Input: 3 kA Peak Current in 25 Turn Air Core Copper Coil))

| | H. Displacement | | | | | | | | | | | | | | |
|---|---|---|---|---|---|---|---|---|---|---|---|---|---|---|---|
| | to Ohmic RMS | | | I. RMS Current | | | J. Peak Current | | | K. RMS Electric | | | L. Peak Electric Field | | |
| | Current Ratio | | | Density (A/m\hat{ }2) | | | Density (A/m\hat{ }2) | | | Field (V/m) | | | (V/m) | | |
| G. Coil | Ex- | Ex- | | Ex- | Ex- | | Ex- | Ex- | | Ex- | Ex- | | Ex- | Ex- | |
| Input | vivo | vivo | In- | vivo | vivo | In- | vivo | vivo | In- | vivo | vivo | In- | vivo | vivo | In- |
| Waveform | 1 | 2 | vivo | 1 | 2 | vivo | 1 | 2 | vivo | 1 | 2 | vivo | 1 | 2 | vivo |
| SP 65 μs | 0.00066 | 0.07 | 0.49 | 4.96 | 2.76 | 6.20 | 27.11 | 15.25 | 31.65 | 17.96 | 21.90 | 6.80 | 98.18 | 119.92 | 42.33 |
| CBP 65 | 0.00065 | 0.07 | 0.48 | 11.15 | 6.19 | 13.66 | 26.81 | 15.06 | 31.06 | 40.39 | 49.27 | 15.41 | 97.10 | 118.41 | 41.29 |
| μs | | | | | | | | | | | | | | | |
| SP 300 μs | 0.00066 | 0.07 | 0.50 | 5.24 | 2.92 | 6.55 | 27.13 | 15.29 | 31.82 | 18.99 | 23.22 | 7.60 | 98.22 | 120.35 | 43.83 |
| CBP 300 | 0.00066 | 0.07 | 0.50 | 5.24 | 2.91 | 6.55 | 27.06 | 15.24 | 31.67 | 18.98 | 23.20 | 7.62 | 97.99 | 119.90 | 43.39 |
| μs | | | | | | | | | | | | | | | |
| SP 600 μs | 0.00066 | 0.07 | 0.50 | 3.71 | 2.06 | 4.64 | 27.13 | 15.29 | 31.82 | 13.44 | 16.43 | 5.40 | 98.22 | 120.35 | 43.83 |
| CBP 600 | 0.00066 | 0.07 | 0.50 | 3.71 | 2.06 | 4.64 | 27.07 | 15.22 | 31.76 | 13.42 | 16.37 | 5.50 | 98.01 | 119.70 | 43.85 |
| μs | | | | | | | | | | | | | | | |
| SP 1000 | 0.00066 | 0.07 | 0.50 | 2.87 | 1.60 | 3.60 | 27.10 | 15.29 | 31.89 | 10.39 | 12.73 | 4.33 | 98.09 | 120.39 | 44.36 |
| μs | | | | | | | | | | | | | | | |
| CBP 1000 | 0.00066 | 0.07 | 0.50 | 2.87 | 1.60 | 3.60 | 27.08 | 15.27 | 31.84 | 10.39 | 12.73 | 4.34 | 98.02 | 120.12 | 44.19 |
| μs | | | | | | | | | | | | | | | |
| SP 2000 | 0.00066 | 0.07 | 0.50 | 2.03 | 1.13 | 2.54 | 27.12 | 15.29 | 31.90 | 7.36 | 9.02 | 3.08 | 98.15 | 120.51 | 44.38 |
| μs | | | | | | | | | | | | | | | |
| CBP 2000 | 0.00066 | 0.07 | 0.50 | 2.03 | 1.13 | 2.54 | 27.11 | 15.27 | 31.87 | 7.36 | 9.02 | 3.08 | 98.11 | 120.26 | 44.29 |
| μs | | | | | | | | | | | | | | | |
| ST1 | 0.00004 | 0.15 | 0.62 | 0.41 | 0.19 | 0.39 | 1.19 | 0.56 | 1.13 | 1.46 | 1.95 | 1.48 | 4.27 | 5.60 | 3.74 |
| ST2 | 0.00009 | 0.12 | 0.49 | 0.92 | 0.45 | 0.98 | 2.86 | 1.43 | 3.08 | 3.33 | 4.25 | 2.32 | 10.33 | 13.08 | 6.41 |
| SW500 | 0.00003 | 0.17 | 0.84 | 0.40 | 0.20 | 0.39 | 0.68 | 0.40 | 1.00 | 1.43 | 2.13 | 2.05 | 2.45 | 3.42 | 3.29 |
| SW5000 | 0.00011 | 0.10 | 0.43 | 4.71 | 2.43 | 5.19 | 6.95 | 3.79 | 8.74 | 17.04 | 21.61 | 8.28 | 25.15 | 31.64 | 11.72 |
| TP 1 | 0.00066 | 0.07 | 0.50 | 3.52 | 1.96 | 4.42 | 26.48 | 15.07 | 32.26 | 12.73 | 15.53 | 4.99 | 95.91 | 116.81 | 40.99 |
| TP 2 | 0.00066 | 0.07 | 0.50 | 3.76 | 2.09 | 4.72 | 26.79 | 15.19 | 32.26 | 13.61 | 16.62 | 5.45 | 97.02 | 118.28 | 42.16 |
| TMS 1 | 0.00047 | 0.08 | 0.47 | 4.08 | 2.19 | 5.00 | 17.75 | 9.88 | 21.61 | 14.75 | 18.19 | 6.67 | 64.25 | 78.81 | 28.37 |
| TMS 2 | 0.00042 | 0.09 | 0.49 | 4.28 | 2.27 | 5.18 | 21.12 | 11.58 | 25.36 | 15.49 | 19.11 | 7.23 | 76.50 | 93.13 | 32.56 |
| TMS 3 | 0.00017 | 0.10 | 0.45 | 2.13 | 1.08 | 2.38 | 6.33 | 3.42 | 8.09 | 7.71 | 9.62 | 4.05 | 22.89 | 28.40 | 10.86 |

The center and lower panels of FIG. 7 show the spatial and temporal composition of the current density in terms of ohmic and displacement currents. Both ex-vivo based current densities demonstrate only minor displacement components. In contrast, the in-vivo current density contains substantial displacement components of comparable magnitude to ohmic components. Ignoring these displacement components leads to inaccurate total current density magnitudes and waveform dynamics. This is seen not only in terms of total magnitude, but also in terms of focality of the current density distribution.

For example, the maximum cortical current density areas (defined as the surface areas on the cortex where the current density was greater than 90% of its maximum value) were 174 mm², 163 mm², and 216 mm² for the two ex-vivo and in-vivo solutions, respectively, demonstrating a greater current spread in the in-vivo tissues (Middle row FIG. 7). FIG. 8 shows the temporal behavior of the induced electric field and current density broken up into components tangential and normal to the gray matter surface. For all of the solutions at the evaluation site, the electric field and current density were primarily composed of vector components tangential to the coil face (approximately aligned with the composite vector, and nearly tangential to the CSF-gray matter boundary at the location of evaluation). However, the waveforms from the in-vivo and ex-vivo measurements had distinct, directionally dependent temporal dynamics; the vector field components showed the greatest variation in the direction approximately normal to the tissue boundaries (FIGS. 7, 8, & Table 3).

These findings were consistent across the 19 distinct stimulation waveforms tested and in each case the displacement currents comprised a significant component of the in-vivo fields, resulting in significantly different current densities, electric field magnitudes, and stimulation waveform shape/dynamics compared to those developed with past ex-vivo impedance values that have been used to develop neurostimulation theory (Significance defined as p<0.01 for Wilcoxon signed-rank tests. See Table 3 above and Table 4 below).

TABLE 4
Average Differences between the in-vivo based solutions and those developed with the relevant ex-vivo set as
indicated below (p values corresponding to signifigance difference tests)

| TMS Field Solutions Along Composite Vector | | | | | TMS Field Solutions Along Normal Vector | | | | |
|---|---|---|---|---|---|---|---|---|---|
| | Ex-vivo 1 | | Ex-vivo 2 | | | Ex-vivo 1 | | Ex-vivo 2 | |
| | Average | p-value | Average | p-value | | Average | p-value | Average | p-value |
| Displacement | 50.89% | 1.09E−04 | 42.19% | 1.09E−04 | Displacement | 51.28% | 1.01E−04 | 42.55% | 1.01E−04 |
| to Ohmic | | | | | to Ohmic | | | | |
| Ratio | | | | | Ratio | | | | |
| RMS Current | 165.61% | 1.82E−04 | 474.63% | 1.32E−04 | RMS Current | 22.25% | 2.12E−04 | 123.52% | 1.31E−04 |
| Density | | | | | Density | | | | |
| Peak Current | 161.68% | 1.32E−04 | 452.38% | 1.32E−04 | Peak Current | 18.37% | 1.54E−04 | 110.87% | 1.31E−04 |
| Density | | | | | Density | | | | |
| RMS Electric | −13.88% | 1.55E−04 | −17.12% | 1.32E−04 | RMS Electric | −57.08% | 2.13E−04 | −65.11% | 1.32E−04 |
| Field | | | | | Field | | | | |
| Peak Electric | −4.96% | 1.32E−04 | −8.08% | 1.32E−04 | Peak Electric | −55.34% | 1.82E−04 | −63.54% | 1.32E−04 |
| Field | | | | | Field | | | | |
| Current Constrained Monopole and Dipole DBS Field | | | | | Voltage Constrained Monopole and Dipole DBS Field | | | | |
| Solutions | | | | | Solutions | | | | |
| | Ex-vivo 1 | | Ex-vivo 2 | | | Ex-vivo 1 | | Ex-vivo 2 | |
| | Average | p-value | Average | p-value | | Average | p-value | Average | p-value |
| Displacement | 64.24% | 1.32E−04 | 52.01% | 1.30E−04 | Displacement | 57.62% | 1.32E−04 | 44.83% | 1.32E−04 |
| to Ohmic | | | | | to Ohmic | | | | |
| Ratio | | | | | Ratio | | | | |
| RMS Electric | −40.00% | 1.32E−04 | −74.98% | 1.32E−04 | RMS Current | 23.53% | 1.26E−01 | 259.09% | 1.32E−04 |
| Field | | | | | Density | | | | |
| Peak Electric | −39.05% | 2.13E−04 | −74.59% | 1.32E−04 | Peak Current | 119.23% | 2.50E−04 | 434.91% | 1.32E−04 |
| Field | | | | | Density | | | | |

Human Motor Neuron Threshold Values

| | |
|---|---|
| Ex-vivo 1 | Ex-vivo 2 |

| | Average | p-value | Average | p-value |
|---|---|---|---|---|
| TMS Coil Current for neuron oriented along composite vector | 56.71% | 6.28E−04 | 58.09% | 3.97E−04 |
| TMS Coil Current for neuron oriented along normal vector | 115.82% | 2.50E−04 | 160.82% | 1.32E−04 |
| DBS Current Constrained Monopole | 35.26% | 2.30E−04 | 232.16% | 1.30E−04 |
| DBS Current Constrained Dipole | 35.45% | 1.82E−04 | 233.47% | 1.30E−04 |

Results. Tissue Effects on the DBS Fields:
We also constructed MRI guided FEMs of the human head to calculate the fields generated during DBS. Time dependent solutions of the voltage constrained DBS field distributions also demonstrated significant differences based on tissue impedance. Spatial and temporal snapshots of the resulting current densities from one stimulation waveform (Charge balanced, 600 microsecond pulse) are shown in FIG. 9. The top panel shows the voltage constrained waveform across a dipole stimulating electrode on the left, and the resulting current waveforms at the dipole center for the in-vivo and ex-vivo impedances on the right. The in-vivo based current density magnitude has a significantly larger initial peak and altered temporal dynamics compared to those developed with the ex-vivo measurements. The center and lower panel show the spatial and temporal composition of the current density at the dipole center in terms of ohmic and displacement components. As with the TMS fields, the displacement current magnitudes are minor relative to the ohmic components for the ex-vivo impedances, but represent a large component of the in-vivo current density (FIG. 9 and Table 5 below).

TABLE 5
1. DBS Field Solutions for a Current Constrained Dipole Source (evaluated along the vector parallel to the
electrode shaft, at center point 0.75 mm from source in Gray Matter (Input: 0.1 mA peak))

| | B. Displacement to | | | | | | | | | Constrained | |
|---|---|---|---|---|---|---|---|---|---|---|---|
| | Ohmic RMS Current | | | C. RMS Electric Field | | | D. Peak Electric Field | | | Current Density | |
| A. Electrode | Ratio | | | (V/m) | | | (V/m) | | | E. RMS | F. Peak |
| Input | | Ex- | In- | Ex- | | In- | Ex- | | | (A/m\hat{ }2) | (A/m\hat{ }2) |
| Waveform | Ex-vivo 1 | vivo 2 | vivo | vivo 1 | vivo 2 | vivo | Ex-vivo 1 | Ex-vivo 2 | In-vivo | All sets | All sets |
| SP 65 μs | 0.00001 | 0.15 | 0.86 | 20.00 | 45.19 | 8.14 | 56.29 | 127.19 | 20.54 | 3.6 | 9 |
| CBP 65 μs | 0.00002 | 0.12 | 0.66 | 36.31 | 80.69 | 12.65 | 45.43 | 102.49 | 16.95 | 6.8 | 9 |
| SP 300 μs | 0.00012 | 0.08 | 0.46 | 56.97 | 134.15 | 27.47 | 71.44 | 172.24 | 39.11 | 8.0 | 9 |
| CBP 300 μs | 0.00014 | 0.08 | 0.40 | 50.10 | 118.21 | 24.90 | 58.51 | 139.95 | 33.53 | 7.1 | 9 |
| SP 600 μs | 0.00005 | 0.10 | 0.64 | 41.09 | 97.73 | 22.88 | 71.44 | 172.24 | 39.11 | 5.7 | 9 |
| CBP 600 μs | 0.00005 | 0.11 | 0.54 | 55.94 | 136.03 | 34.88 | 64.56 | 159.01 | 47.11 | 7.2 | 9 |
| SP 1000 μs | 0.00004 | 0.12 | 0.66 | 67.06 | 166.16 | 47.79 | 82.34 | 210.36 | 68.21 | 8.0 | 9 |
| CBP 1000 μs | 0.00003 | 0.13 | 0.67 | 60.51 | 150.96 | 45.39 | 69.44 | 175.29 | 60.80 | 7.2 | 9 |
| SP 2000 μs | 0.00002 | 0.13 | 0.88 | 71.50 | 182.74 | 63.60 | 87.29 | 231.33 | 90.49 | 7.8 | 9 |
| CBP 2000 μs | 0.00002 | 0.14 | 0.77 | 66.63 | 172.50 | 63.36 | 76.10 | 199.15 | 83.62 | 7.3 | 9 |
| ST1 | 0.00001 | 0.16 | 1.00 | 36.77 | 90.76 | 26.63 | 66.69 | 159.05 | 37.23 | 4.4 | 9 |
| ST2 | 0.00002 | 0.17 | 0.91 | 30.88 | 73.57 | 17.16 | 58.61 | 135.01 | 24.70 | 4.2 | 9 |
| SW500 | 0.00001 | 0.17 | 0.89 | 44.79 | 107.42 | 25.87 | 71.74 | 174.89 | 45.98 | 5.8 | 9 |
| SW5000 | 0.00010 | 0.10 | 0.44 | 32.61 | 71.18 | 10.25 | 50.15 | 111.69 | 17.57 | 6.3 | 9 |
| TP 1 | 0.00011 | 0.11 | 0.48 | 15.99 | 35.60 | 5.43 | 39.23 | 86.36 | 13.38 | 2.9 | 9 |
| TP 2 | 0.00007 | 0.13 | 0.57 | 27.36 | 62.48 | 10.84 | 44.93 | 101.22 | 17.28 | 4.3 | 9 |
| TMS 1 | 0.00007 | 0.10 | 0.45 | 31.71 | 72.35 | 12.70 | 48.88 | 108.86 | 17.25 | 5.2 | 9 |
| TMS 2 | 0.00010 | 0.11 | 0.45 | 25.09 | 55.08 | 7.91 | 43.28 | 95.05 | 14.46 | 4.7 | 9 |
| TMS 3 | 0.00007 | 0.11 | 0.47 | 22.63 | 50.89 | 8.52 | 52.39 | 117.68 | 18.96 | 3.9 | 9 |

2. DBS Field Solutions for a Voltage Constrained Dipole Source (evaluated along the vector parallel to the
electrode shaft, at center point 0.75 mm from source in Gray Matter (Input: 0.2 V peak to peak))

| | B2. Displacement to | | | | | | | | | Constrained Electric | |
|---|---|---|---|---|---|---|---|---|---|---|---|
| | Ohmic RMS Current | | | C2. RMS Current | | | D2. Peak Current | | | Field | |
| A2. Electrode | Ratio | | | Density (A/m\hat{ }2) | | | Density (A/m\hat{ }2) | | | E2. RMS | F2. Peak |
| Input | | Ex- | In- | Ex- | Ex- | In- | Ex- | Ex- | | (V/m) | (V/m) |
| Waveform | Ex-vivo 1 | vivo 2 | vivo | vivo 1 | vivo 2 | vivo | vivo 1 | vivo 2 | In-vivo | All sets | All sets |
| SP 65 μs | 0.00017 | 0.09 | 0.46 | 12.69 | 5.00 | 25.95 | 35.05 | 14.91 | 90.50 | 46.8 | 118 |
| CBP 65 μs | 0.00020 | 0.09 | 0.47 | 24.18 | 9.64 | 51.92 | 33.99 | 14.50 | 90.03 | 89.5 | 118 |
| SP 300 μs | 0.00008 | 0.10 | 0.54 | 28.95 | 9.97 | 33.08 | 35.21 | 15.04 | 84.85 | 105.9 | 118 |
| CBP 300 μs | 0.00009 | 0.12 | 0.55 | 25.79 | 8.98 | 32.27 | 35.20 | 14.94 | 84.93 | 93.7 | 118 |
| SP 600 μs | 0.00008 | 0.12 | 0.54 | 20.62 | 7.15 | 24.35 | 35.21 | 15.04 | 84.85 | 75.4 | 118 |
| CBP 600 μs | 0.00007 | 0.14 | 0.61 | 26.02 | 8.46 | 24.79 | 35.30 | 15.01 | 84.65 | 94.4 | 118 |
| SP 1000 μs | 0.00005 | 0.14 | 0.65 | 28.91 | 8.89 | 20.70 | 34.75 | 15.09 | 85.30 | 107.0 | 118 |
| CBP 1000 μs | 0.00005 | 0.16 | 0.66 | 26.20 | 8.06 | 20.31 | 35.45 | 15.08 | 84.93 | 94.7 | 118 |
| SP 2000 μs | 0.00003 | 0.16 | 0.72 | 28.41 | 8.17 | 15.77 | 34.10 | 15.11 | 86.08 | 107.2 | 118 |
| CBP 2000 μs | 0.00004 | 0.19 | 0.73 | 26.37 | 7.51 | 15.50 | 35.61 | 15.21 | 85.68 | 94.9 | 118 |
| ST1 | 0.00001 | 0.15 | 0.76 | 16.12 | 5.05 | 10.48 | 32.14 | 10.73 | 25.83 | 59.4 | 118 |
| ST2 | 0.00003 | 0.13 | 0.57 | 15.25 | 5.20 | 15.98 | 32.35 | 11.68 | 40.73 | 55.6 | 118 |
| SW500 | 0.00001 | 0.17 | 0.88 | 20.90 | 6.81 | 13.44 | 32.09 | 10.58 | 21.43 | 75.9 | 118 |
| SW5000 | 0.00010 | 0.10 | 0.43 | 22.86 | 9.23 | 50.64 | 32.52 | 13.12 | 73.57 | 82.8 | 118 |
| TP 1 | 0.00016 | 0.11 | 0.47 | 10.29 | 4.04 | 20.09 | 32.87 | 14.10 | 86.64 | 37.7 | 117 |
| TP 2 | 0.00011 | 0.12 | 0.51 | 15.64 | 5.81 | 23.53 | 34.11 | 14.56 | 86.03 | 57.0 | 117 |
| TMS 1 | 0.00010 | 0.11 | 0.49 | 18.70 | 7.04 | 32.01 | 32.85 | 12.91 | 73.05 | 67.8 | 118 |
| TMS 2 | 0.00012 | 0.10 | 0.45 | 17.15 | 6.87 | 36.50 | 33.85 | 14.38 | 89.16 | 62.2 | 118 |
| TMS 3 | 0.00007 | 0.11 | 0.46 | 14.04 | 5.41 | 25.68 | 32.45 | 12.40 | 56.02 | 50.9 | 118 |

3. DBS Field Solutions for a Current Constrained Monopole Source (evaluated along the vector
parallel to the electrode shaft, at point 0.75 mm from source in Gray Matter (Input: 0.1 mA peak))

| | H. Displacement to | | | | | | | | | Constrained | |
|---|---|---|---|---|---|---|---|---|---|---|---|
| | Ohmic RMS | | | I. RMS Electric | | | J. Peak Electric Field | | | Current Density | |
| G. Electrode | Current Ratio | | | Field (V/m) | | | (V/m) | | | K. RMS | L. Peak |
| Input | | Ex- | In- | Ex- | Ex- | In- | Ex- | Ex- | | (A/m\hat{ }2) | (A/m\hat{ }2) |
| Waveform | Ex-vivo 1 | vivo 2 | vivo | vivo 1 | vivo 2 | vivo | vivo 1 | vivo 2 | In-vivo | All sets | All sets |
| SP 65 μs | 0.00001 | 0.15 | 0.86 | 9.08 | 20.53 | 3.70 | 25.57 | 57.78 | 9.33 | 1.6 | 4 |
| CBP 65 μs | 0.00002 | 0.12 | 0.66 | 16.49 | 36.65 | 5.75 | 20.63 | 46.55 | 7.70 | 3.1 | 4 |
| SP 300 μs | 0.00012 | 0.08 | 0.46 | 25.88 | 60.94 | 12.48 | 32.45 | 78.24 | 17.77 | 3.6 | 4 |
| CBP 300 μs | 0.00014 | 0.08 | 0.40 | 22.76 | 53.69 | 11.31 | 26.58 | 63.57 | 15.23 | 3.2 | 4 |
| SP 600 μs | 0.00005 | 0.10 | 0.64 | 18.66 | 44.39 | 10.39 | 32.45 | 78.24 | 17.77 | 2.6 | 4 |
| CBP 600 μs | 0.00005 | 0.11 | 0.54 | 25.41 | 61.79 | 15.85 | 29.33 | 72.23 | 21.40 | 3.3 | 4 |
| SP 1000 μs | 0.00004 | 0.12 | 0.66 | 30.46 | 75.48 | 21.71 | 37.40 | 95.55 | 30.98 | 3.6 | 4 |
| CBP 1000 μs | 0.00003 | 0.13 | 0.67 | 27.48 | 68.57 | 20.62 | 31.54 | 79.63 | 27.62 | 3.3 | 4 |
| SP 2000 μs | 0.00002 | 0.13 | 0.88 | 32.48 | 83.01 | 28.89 | 39.65 | 105.08 | 41.11 | 3.6 | 4 |
| CBP 2000 μs | 0.00002 | 0.14 | 0.77 | 30.27 | 78.35 | 28.78 | 34.57 | 90.46 | 37.98 | 3.3 | 4 |
| ST1 | 0.00001 | 0.16 | 1.00 | 16.70 | 41.23 | 12.10 | 30.29 | 72.25 | 16.91 | 2.0 | 4 |
| ST2 | 0.00002 | 0.17 | 0.91 | 14.03 | 33.42 | 7.80 | 26.62 | 61.33 | 11.22 | 1.9 | 4 |
| SW500 | 0.00001 | 0.17 | 0.89 | 20.34 | 48.79 | 11.75 | 32.59 | 79.44 | 20.89 | 2.6 | 4 |
| SW5000 | 0.00010 | 0.10 | 0.44 | 14.81 | 32.33 | 4.66 | 22.78 | 50.73 | 7.98 | 2.9 | 4 |
| TP 1 | 0.00011 | 0.11 | 0.48 | 7.26 | 16.17 | 2.47 | 17.82 | 39.23 | 6.08 | 1.3 | 4 |
| TP 2 | 0.00007 | 0.13 | 0.57 | 12.43 | 28.38 | 4.92 | 20.41 | 45.98 | 7.85 | 2.0 | 4 |
| TMS 1 | 0.00007 | 0.10 | 0.45 | 14.40 | 32.86 | 5.77 | 22.20 | 49.45 | 7.83 | 2.3 | 4 |
| TMS 2 | 0.00010 | 0.11 | 0.45 | 11.40 | 25.02 | 3.59 | 19.66 | 43.18 | 6.57 | 2.1 | 4 |
| TMS 3 | 0.00007 | 0.11 | 0.47 | 10.28 | 23.12 | 3.87 | 23.80 | 53.45 | 8.61 | 1.8 | 4 |

4. DBS Field Solutions for a Voltage Constrained Monopole Source (evaluated along
the vector parallel to the electrode shaft, at point 0.75 mm from source in Gray Matter
(Input: 0.2 V peak to peak))

| | H2. Displacement | | | I2. RMS | | | | | | Constrained | |
|---|---|---|---|---|---|---|---|---|---|---|---|
| | to Ohmic RMS | | | Current Density | | | J2. Peak Current | | | Electric Field | |
| G2. Electrode | Current Ratio | | | (A/m\hat{ }2) | | | Density (A/m\hat{ }2) | | | K2. RMS | L2. Peak |
| Input | | Ex- | In- | Ex- | Ex- | In- | Ex- | Ex- | In- | (V/m) | (V/m) |
| Waveform | Ex-vivo 1 | vivo 2 | vivo | vivo 1 | vivo 2 | vivo | vivo 1 | vivo 2 | vivo | All sets | All sets |
| SP 65 μs | 0.00017 | 0.09 | 0.46 | 4.25 | 1.67 | 8.68 | 11.73 | 4.99 | 30.28 | 15.7 | 39 |
| CBP 65 μs | 0.00020 | 0.09 | 0.47 | 8.09 | 3.22 | 17.37 | 11.37 | 4.85 | 30.12 | 29.9 | 39 |
| SP 300 μs | 0.00008 | 0.10 | 0.54 | 9.69 | 3.34 | 11.07 | 11.78 | 5.03 | 28.39 | 35.4 | 39 |
| CBP 300 μs | 0.00009 | 0.12 | 0.55 | 8.63 | 3.00 | 10.80 | 11.78 | 5.00 | 28.42 | 31.4 | 39 |
| SP 600 μs | 0.00008 | 0.12 | 0.54 | 6.90 | 2.39 | 8.15 | 11.78 | 5.03 | 28.39 | 25.2 | 39 |
| CBP 600 μs | 0.00007 | 0.14 | 0.61 | 8.70 | 2.83 | 8.29 | 11.81 | 5.02 | 28.32 | 31.6 | 39 |
| SP 1000 μs | 0.00005 | 0.14 | 0.65 | 9.67 | 2.97 | 6.93 | 11.63 | 5.05 | 28.54 | 35.8 | 39 |
| CBP 1000 | 0.00005 | 0.16 | 0.66 | 8.76 | 2.70 | 6.80 | 11.86 | 5.04 | 28.42 | 31.7 | 39 |
| μs | | | | | | | | | | | |
| SP 2000 μs | 0.00003 | 0.16 | 0.72 | 9.50 | 2.73 | 5.28 | 11.41 | 5.06 | 28.80 | 35.9 | 39 |
| CBP 2000 | 0.00004 | 0.19 | 0.73 | 8.82 | 2.51 | 5.19 | 11.91 | 5.09 | 28.67 | 31.7 | 39 |
| μs | | | | | | | | | | | |
| ST1 | 0.00001 | 0.15 | 0.76 | 5.39 | 1.69 | 3.51 | 10.75 | 3.59 | 8.64 | 19.9 | 39 |
| ST2 | 0.00003 | 0.13 | 0.57 | 5.10 | 1.74 | 5.35 | 10.82 | 3.91 | 13.63 | 18.6 | 39 |
| SW500 | 0.00001 | 0.17 | 0.88 | 6.99 | 2.28 | 4.50 | 10.74 | 3.54 | 7.17 | 25.4 | 39 |
| SW5000 | 0.00010 | 0.10 | 0.43 | 7.65 | 3.09 | 16.94 | 10.88 | 4.39 | 24.62 | 27.7 | 39 |
| TP 1 | 0.00016 | 0.11 | 0.47 | 3.44 | 1.35 | 6.72 | 11.00 | 4.72 | 28.99 | 12.6 | 39 |
| TP 2 | 0.00011 | 0.12 | 0.51 | 5.23 | 1.94 | 7.87 | 11.41 | 4.87 | 28.78 | 19.1 | 39 |
| TMS 1 | 0.00010 | 0.11 | 0.49 | 6.26 | 2.35 | 10.71 | 10.99 | 4.32 | 24.44 | 22.7 | 39 |
| TMS 2 | 0.00012 | 0.10 | 0.45 | 5.74 | 2.30 | 12.21 | 11.33 | 4.81 | 29.83 | 20.8 | 39 |
| TMS 3 | 0.00007 | 0.11 | 0.46 | 4.70 | 1.81 | 8.59 | 10.86 | 4.15 | 18.74 | 17.0 | 39 |

Additionally, we determined the electric fields and current densities for current constrained DBS stimulation waveforms; time dependent solutions of the DBS generated field distributions demonstrated analogous differences based on the tissue impedances. The resultant electric fields were decreased in magnitude in the in-vivo solutions compared to the ex-vivo solutions. By constraint, the total current density magnitude was the same across solutions; but, there were significant differences in the current density composition across the solutions. As in the other systems studied, the in-vivo solutions demonstrated significant displacement currents in the tissues, while the ex-vivo values led to only minor displacement currents (Table 5). Both the voltage and current constrained DBS field solutions were confined to the region of gray matter in which the electrodes were placed and negligible at tissue boundaries. Thus there were no effects at the tissue boundaries in these solutions (FIG. 9 and Table 5).

As with TMS, these findings were consistent across the 19 distinct stimulation waveforms tested, for both monopole and dipole electrodes, and in each case, the displacement currents comprised a significant component of the in-vivo fields, resulting in significantly different current densities, electric field magnitudes, and stimulation waveform shape/dynamics in a source dependent manner compared to those developed with past ex-vivo impedance values (Tables 4 and 5 above).

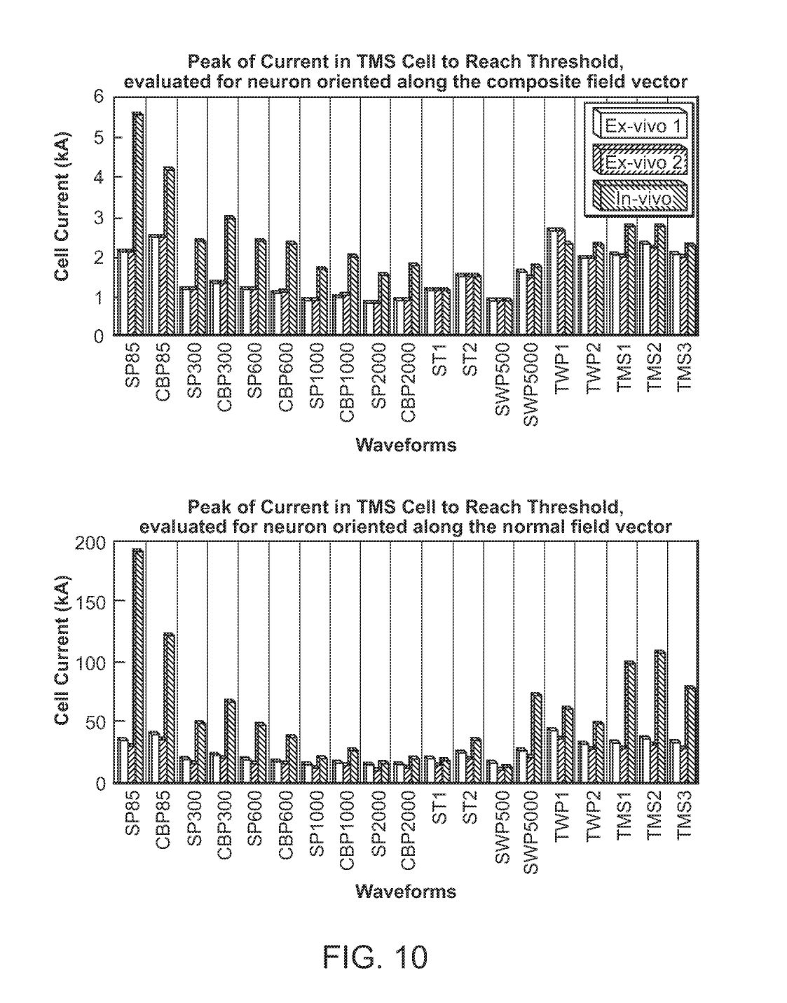

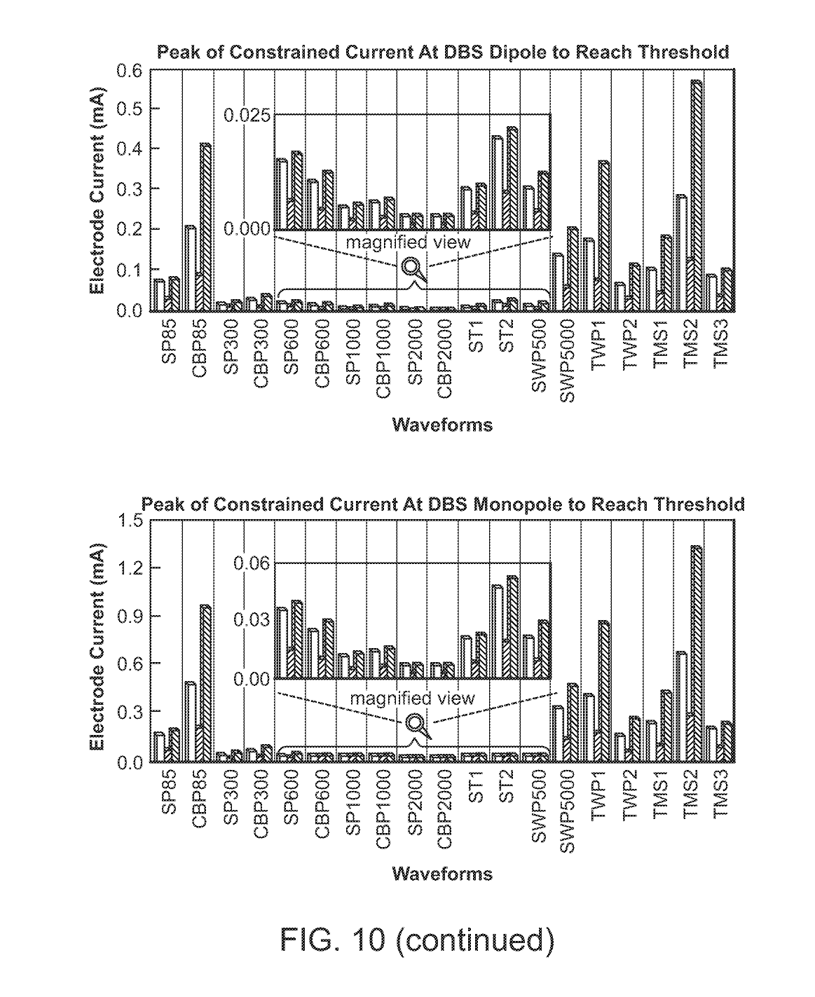

Results. Tissue Effects on Neural Response:
We developed conductance-based models of the human motor neuron, driven by the fields derived from the MRI guided FEMs. We compared the neurostimulation thresholds and membrane dynamics for these neurons responding to the external stimulating fields (for both TMS and DBS sources) in tissues with in-vivo and ex-vivo properties. The thresholds are tabulated for each stimulation waveform and condition in FIG. 10. As described in the figure, the predicted stimulation thresholds were higher for nearly all stimulation conditions in the in-vivo systems due to the increased tissue impedances and resulting attenuation of the electric fields. In comparison to published experimental neural data, the frequency independent ex-vivo (ex-vivo 1 solutions) and the frequency dependent in-vivo thresholds were both within published ranges, but the in-vivo field waveforms demonstrated the greatest similarity to direct waveform measurements with similar driving sources (Tehovnik, Electrical stimulation of neural tissue to evoke behavioral responses, J Neurosci Methods, 65, (1), 1-17, 1996), (Tay, Measurement of magnetically induced current density in saline and in vivo, Engineering in Medicine and Biology Society, 1989. Images of the Twenty-First Century, Proceedings of the Annual International Conference of the IEEE, 4, (1167-1168, 1989), (Tay, Measurement of current density distribution induced in vivo during magnetic stimulation, PhD, (211, 1992) (FIGS. 7-9, Tables 3 and 5 above and Table 6 below (in table 6, neg is used as an abbreviation for negligible)). Finally, the dynamics of the membrane response were effectively altered in the in-vivo solutions, due to changes in the external driving field; potassium and sodium ionic flow across the membrane were generally decreased compared to the ex-vivo based dynamics for neurons in a sub-threshold state.

TABLE 6
Human Motor Neuron Initial Segment Thresholds Evaluated as a Function of the Source Inputs

| | B. Peak | | | | | | | | |
|---|---|---|---|---|---|---|---|---|---|
| | Value of | | | | | | | | |
| | Current in | | | | | | | | |
| | TMS Coil to | | | | | | | | |
| | Reach | | | C. Peak Value of | | | | | |
| | Threshold | | | Current in TMS | | | | | |
| | (kA), | | | Coil to Reach | | | | | |
| | evaluated for | | | Threshold (kA), | | | | | |
| | neuron | | | evaluated for | | | D. Peak Value of | | |
| | oriented along | | | neuron oriented | | | Constrained Current | | |
| | the composite | | | along the normal- | | | Injected At DBS | | |
| | field vector | | | field vector (25 turn | | | Dipole to Reach | | |
| A. Source | (25 turn coil) | | | coil) | | | Threshold (mA) | | |
| Input | Ex- | Ex- | In- | Ex- | Ex- | In- | Ex- | Ex- | In- |
| Waveform | vivo 1 | vivo 2 | vivo | vivo 1 | vivo 2 | vivo | vivo 1 | vivo 2 | vivo |
| SP 65 μs | 2.13 | 2.12 | 5.58 | 35.10 | 29.84 | 194.28 | 7.11E−02 | 2.80E−02 | 7.82E−02 |
| CBP 65 | 2.51 | 2.50 | 4.19 | 41.20 | 35.16 | 124.03 | 2.06E−01 | 8.78E−02 | 4.11E−01 |
| μs | | | | | | | | | |
| SP 300 μs | 1.19 | 1.19 | 2.37 | 19.61 | 16.31 | 49.27 | 1.56E−02 | 6.16E−03 | 1.70E−02 |
| CBP 300 | 1.36 | 1.37 | 2.96 | 22.31 | 18.88 | 68.17 | 2.44E−02 | 9.88E−03 | 3.32E−02 |
| μs | | | | | | | | | |
| SP 600 μs | 1.19 | 1.19 | 2.37 | 19.61 | 16.31 | 49.27 | 1.56E−02 | 6.16E−03 | 1.70E−02 |
| CBP 600 | 1.12 | 1.15 | 2.30 | 18.34 | 15.55 | 39.14 | 1.04E−02 | 4.15E−03 | 1.24E−02 |
| μs | | | | | | | | | |
| SP 1000 | 0.91 | 0.90 | 1.67 | 15.26 | 11.98 | 20.82 | 4.92E−03 | 1.96E−03 | 5.30E−03 |
| μs | | | | | | | | | |
| CBP 1000 | 1.00 | 1.05 | 2.01 | 16.78 | 14.08 | 26.77 | 5.97E−03 | 2.34E−03 | 6.35E−03 |
| μs | | | | | | | | | |
| SP 2000 | 0.86 | 0.79 | 1.55 | 14.31 | 10.47 | 16.02 | 2.82E−03 | 1.10E−03 | 2.82E−03 |
| μs | | | | | | | | | |
| CBP 2000 | 0.92 | 0.94 | 1.77 | 15.31 | 12.53 | 19.62 | 3.10E−03 | 1.20E−03 | 2.91E−03 |
| μs | | | | | | | | | |
| ST1 | 1.17 | 1.14 | 1.16 | 19.44 | 14.75 | 18.95 | 8.92E−03 | 3.49E−03 | 9.49E−03 |
| ST2 | 1.50 | 1.47 | 1.52 | 24.90 | 19.87 | 35.71 | 2.06E−02 | 8.06E−03 | 2.24E−02 |
| SW500 | 0.90 | 0.88 | 0.90 | 15.92 | 11.08 | 12.52 | 9.11E−03 | 3.68E−03 | 1.24E−02 |
| SW5000 | 1.63 | 1.48 | 1.73 | 26.43 | 21.75 | 71.98 | 1.38E−01 | 5.68E−02 | 2.01E−01 |
| TP 1 | 2.66 | 2.67 | 2.32 | 43.59 | 37.35 | 61.39 | 1.74E−01 | 7.45E−02 | 3.68E−01 |
| TP 2 | 1.97 | 1.99 | 2.29 | 32.45 | 27.60 | 49.11 | 6.29E−02 | 2.62E−02 | 1.11E−01 |
| TMS 1 | 2.06 | 2.02 | 2.77 | 34.06 | 28.56 | 98.59 | 1.01E−01 | 4.25E−02 | 1.82E−01 |
| TMS 2 | 2.32 | 2.22 | 2.74 | 38.24 | 31.77 | 109.08 | 2.84E−01 | 1.23E−01 | 5.73E−01 |
| TMS 3 | 2.08 | 2.01 | 2.26 | 34.42 | 28.41 | 79.00 | 8.48E−02 | 3.38E−02 | 9.67E−02 |

| | | | | F. Peak | G. Peak |
|---|---|---|---|---|---|
| | | | | Constrained | Constrained |
| | E. Peak Value of | | | Dipole | Monopole |
| | Constrained Current | | | Voltage to | Voltage to |
| | Injected At Monopole | | | Reach | Reach |
| | to Reach Threshold | | | Threshold | Threshold |
| A. Source | (mA) | | | (V) | (V) |
| Input | Ex- | Ex- | | All | All |
| Waveform | vivo 1 | vivo 2 | In-vivo | Sets | Sets |
| SP 65 μs | 1.66E−01 | 6.56E−02 | 1.83E−01 | 9.38E−02 | 1.49E−01 |
| CBP 65 | 4.82E−01 | 2.06E−01 | 9.61E−01 | 2.16E−01 | 3.43E−01 |
| μs | | | | | |
| SP 300 μs | 3.65E−02 | 1.44E−02 | 3.99E−02 | 2.07E−02 | 3.30E−02 |
| CBP 300 | 5.71E−02 | 2.31E−02 | 7.78E−02 | 3.06E−02 | 4.85E−02 |
| μs | | | | | |
| SP 600 μs | 3.65E−02 | 1.44E−02 | 3.99E−02 | 2.07E−02 | 3.30E−02 |
| CBP 600 | 2.46E−02 | 9.68E−03 | 2.89E−02 | 1.40E−02 | 2.22E−02 |
| μs | | | | | |
| SP 1000 | 1.16E−02 | 4.53E−03 | 1.24E−02 | 6.78E−03 | 1.07E−02 |
| μs | | | | | |
| CBP 1000 | 1.39E−02 | 5.39E−03 | 1.48E−02 | 8.31E−03 | 1.32E−02 |
| μs | | | | | |
| SP 2000 | 6.54E−03 | 2.53E−03 | 6.63E−03 | 3.92E−03 | 6.16E−03 |
| μs | | | | | |
| CBP 2000 | 7.20E−02 | 2.72E−03 | 6.92E−03 | 4.49E−03 | 7.20E−03 |
| μs | | | | | |
| ST1 | 2.08E−02 | 8.16E−03 | 2.22E−02 | 1.21E−02 | 1.92E−02 |
| ST2 | 4.80E−02 | 1.88E−02 | 5.24E−02 | 2.74E−02 | 4.36E−02 |
| SW500 | 2.13E−02 | 8.64E−03 | 2.89E−02 | 1.15E−02 | 1.85E−02 |
| SW5000 | 3.23E−01 | 1.33E−01 | 4.71E−01 | 1.53E−01 | 2.44E−01 |
| TP 1 | 4.08E−01 | 1.74E−01 | 8.63E−01 | 1.82E−01 | 2.90E−01 |
| TP 2 | 1.47E−01 | 6.15E−02 | 2.60E−01 | 7.24E−02 | 1.15E−01 |
| TMS 1 | 2.37E−01 | 9.96E−02 | 4.26E−01 | 1.14E−01 | 1.80E−01 |
| TMS 2 | 6.66E−01 | 2.89E−01 | 1.34E+00 | 2.76E−01 | 4.39E−01 |
| TMS 3 | 1.99E−01 | 7.93E−02 | 2.26E−01 | 1.04E−01 | 1.66E−01 |

| | B1. Peak | | | | | | | | |
|---|---|---|---|---|---|---|---|---|---|
| | Value of | | | | | | | | |
| | Current in | | | | | | | | |
| | TMS Coil to | | | | | | | | |
| | Reach | | | | | | | | |
| | Threshold | | | C1. Peak Value of | | | | | |
| | (kA), | | | Current in TMS | | | | | |
| | evaluated for | | | Coil to Reach | | | | | |
| | neuron | | | Threshold (kA), | | | | | |
| | oriented along | | | evaluated for | | | D1. Peak Value of | | |
| | the composite | | | neuron oriented | | | Constrained Current | | |
| | field vector, | | | along the normal- | | | Injected At DBS | | |
| | as permittivity | | | field vector, as | | | Dipole to Reach | | |
| | approaches | | | permittivity | | | Threshold (mA), as | | |
| | zero for the | | | approaches zero for | | | permittivity | | |
| | system (25 | | | the system (25 turn | | | approaches zero for | | |
| A1. Source | turn coil) | | | coil) | | | the system | | |
| Input | Ex- | Ex- | In- | Ex- | Ex- | In- | Ex- | Ex- | In- |
| Waveform | vivo 1 | vivo 2 | vivo | vivo 1 | vivo 2 | vivo | vivo 1 | vivo 2 | vivo |
| SP 65 μs | 2.13 | 2.13 | 5.47 | 35.09 | 30.72 | 179.26 | 7.11E−02 | 2.78E−02 | 5.39E−02 |
| CBP 65 | 2.50 | 2.51 | 4.17 | 41.18 | 36.18 | 98.23 | 2.06E−01 | 8.74E−02 | 3.38E−01 |
| μs | | | | | | | | | |
| SP 300 μs | 1.19 | 1.19 | 2.38 | 19.61 | 15.78 | 38.28 | 1.56E−02 | 6.06E−03 | 1.18E−02 |
| CBP 300 | 1.36 | 1.37 | 2.97 | 22.31 | 18.45 | 52.80 | 2.44E−02 | 9.78E−03 | 2.48E−02 |
| μs | | | | | | | | | |
| SP 600 μs | 1.19 | 1.19 | 2.38 | 19.61 | 15.78 | 38.28 | 1.56E−02 | 6.06E−03 | 1.18E−02 |
| CBP 600 | 1.12 | 1.15 | 2.43 | 18.35 | 16.08 | 31.10 | 1.04E−02 | 4.15E−03 | 8.83E−03 |
| μs | | | | | | | | | |
| SP 1000 | 0.91 | 0.90 | 1.69 | 15.26 | 11.86 | 16.73 | 4.92E−03 | 1.96E−03 | 3.58E−03 |
| μs | | | | | | | | | |
| CBP 1000 | 1.00 | 1.06 | 2.04 | 16.78 | 14.95 | 21.31 | 5.97E−03 | 2.25E−03 | 4.44E−03 |
| μs | | | | | | | | | |
| SP 2000 | 0.86 | 0.78 | 1.54 | 14.31 | 9.46 | 13.13 | 2.82E−03 | 1.10E−03 | 1.96E−03 |
| μs | | | | | | | | | |
| CBP 2000 | 0.92 | 0.94 | 1.78 | 15.31 | 8.16 | 15.93 | 3.10E−03 | 1.20E−03 | 2.06E−03 |
| μs | | | | | | | | | |
| ST1 | 1.17 | 1.13 | 1.20 | 19.45 | 14.31 | 15.06 | 8.92E−03 | 3.49E−03 | 6.54E−03 |
| ST2 | 1.50 | 1.46 | 1.52 | 24.92 | 20.09 | 29.50 | 2.06E−02 | 7.97E−03 | 1.54E−02 |
| SW500 | 0.90 | 0.88 | 0.90 | 15.92 | 10.82 | 10.15 | 9.11E−03 | 3.68E−03 | 9.30E−03 |
| SW5000 | 1.62 | 1.59 | 1.74 | 26.60 | 21.46 | 75.66 | 1.38E−01 | 5.65E−02 | 1.51E−01 |
| TP 1 | 2.66 | 2.67 | 2.30 | 43.66 | 40.80 | 50.43 | 1.74E−01 | 7.42E−02 | 3.10E−01 |
| TP 2 | 1.97 | 1.98 | 2.33 | 32.46 | 28.54 | 39.93 | 6.29E−02 | 2.61E−02 | 8.98E−02 |
| TMS 1 | 2.06 | 2.03 | 2.74 | 34.09 | 30.02 | 89.71 | 1.01E−01 | 4.24E−02 | 1.46E−01 |
| TMS 2 | 2.31 | 2.27 | 2.73 | 38.31 | 32.80 | 97.39 | 2.84E−01 | 1.23E−01 | 4.15E−01 |
| TMS 3 | 2.07 | 2.03 | 2.25 | 34.52 | 31.48 | 76.15 | 8.49E−02 | 3.36E−02 | 6.67E−02 |

| | | | | F1. Peak | |
|---|---|---|---|---|---|
| | | | | Constrained | G1. Peak |
| | | | | Dipole | Monopole |
| | | | | Voltage to | Voltage to |
| | E1. Peak Value of | | | Reach | Reach |
| | Constrained Current | | | Threshold | Threshold |
| | Injected At Monopole | | | (V) as | (V), as |
| | to Reach Threshold | | | permittivity | permittivity |
| | (mA), as permittivity | | | approaches | approaches |
| | approaches zero for the | | | zero for the | zero for the |
| A1. Source | system | | | system | system |
| Input | Ex- | Ex- | | All | All |
| Waveform | vivo 1 | vivo 2 | In-vivo | Sets | Sets |
| SP 65 μs | 1.67E−01 | 6.51E−02 | 1.26E−01 | 9.38E−02 | 1.49E−01 |
| CBP 65 | 4.82E−01 | 2.05E−01 | 7.91E−01 | 2.16E−01 | 3.43E−01 |
| μs | | | | | |
| SP 300 μs | 3.65E−02 | 1.43E−02 | 2.75E−02 | 2.07E−02 | 3.30E−02 |
| CBP 300 | 5.71E−02 | 2.29E−02 | 5.79E−02 | 3.06E−02 | 4.85E−02 |
| μs | | | | | |
| SP 600 μs | 3.65E−02 | 1.43E−02 | 2.75E−02 | 2.07E−02 | 3.30E−02 |
| CBP 600 | 2.46E−02 | 9.68E−03 | 2.07E−02 | 1.40E−02 | 2.22E−02 |
| μs | | | | | |
| SP 1000 | 1.16E−02 | 4.44E−03 | 8.44E−03 | 6.78E−03 | 1.07E−02 |
| μs | | | | | |
| CBP 1000 | 1.39E−02 | 5.39E−03 | 1.04E−02 | 8.31E−03 | 1.32E−02 |
| μs | | | | | |
| SP 2000 | 6.54E−03 | 2.53E−03 | 4.53E−03 | 3.92E−03 | 6.16E−03 |
| μs | | | | | |
| CBP 2000 | 7.20E−03 | 2.72E−03 | 4.73E−03 | 4.49E−03 | 7.20E−03 |
| μs | | | | | |
| ST1 | 2.08E−02 | 8.06E−03 | 1.52E−02 | 1.21E−02 | 1.92E−02 |
| ST2 | 4.80E−02 | 1.87E−02 | 3.61E−02 | 2.74E−02 | 4.36E−02 |
| SW500 | 2.13E−02 | 8.54E−03 | 2.17E−02 | 1.15E−02 | 1.85E−02 |
| SW5000 | 3.23E−01 | 1.32E−01 | 3.55E−01 | 1.53E−01 | 2.44E−01 |
| TP 1 | 4.08E−01 | 1.74E−01 | 7.26E−01 | 1.82E−01 | 2.90E−01 |
| TP 2 | 1.47E−01 | 6.12E−02 | 2.10E−01 | 7.24E−02 | 1.15E−01 |
| TMS 1 | 2.37E−01 | 9.92E−02 | 3.42E−01 | 1.14E−01 | 1.80E−01 |
| TMS 2 | 6.66E−01 | 2.88E−01 | 9.73E−01 | 2.76E−01 | 4.39E−01 |
| TMS 3 | 1.99E−01 | 7.88E−02 | 1.56E−01 | 1.04E−01 | 1.66E−01 |

| | B2. Peak | | | | | | | | |
|---|---|---|---|---|---|---|---|---|---|
| | Value of | | | | | | | | |
| | Current in | | | | | | | | |
| | TMS Coil to | | | | | | | | |
| | Reach | | | | | | | | |
| | Threshold | | | | | | | | |
| | (kA), | | | C2. Peak Value of | | | | | |
| | evaluated for | | | Current in TMS | | | | | |
| | neuron | | | Coil to Reach | | | | | |
| | oriented along | | | Threshold (kA), | | | | | |
| | the composite | | | evaluated for | | | D2. Peak Value of | | |
| | field vector, | | | neuron oriented | | | Constrained Current | | |
| | as | | | along the normal- | | | Injected At DBS | | |
| | conductivity | | | field vector, as | | | Dipole to Reach | | |
| | approaches | | | conductivity | | | Threshold (mA), as | | |
| | zero for the | | | approaches zero for | | | conductivity | | |
| | system (25 | | | the system (25 turn | | | approaches zero for | | |
| A2. Source | turn coil) | | | coil) | | | the system | | |
| Input | Ex- | Ex- | In- | Ex- | Ex- | In- | Ex- | Ex- | In- |
| Waveform | vivo 1 | vivo 2 | vivo | vivo 1 | vivo 2 | vivo | vivo 1 | vivo 2 | vivo |
| SP 65 μs | 2.32 | 2.59 | 6.60 | 64.21 | 52.46 | 897.11 | neg | 2.25E−03 | 4.95E−02 |
| CBP 65 | 2.73 | 3.03 | 4.84 | 75.42 | 56.17 | 613.66 | neg | 6.35E−03 | 2.05E−01 |
| μs | | | | | | | | | |
| SP 300 μs | 1.30 | 1.48 | 2.79 | 35.99 | 43.47 | 376.08 | neg | 5.29E−04 | 1.08E−02 |
| CBP 300 | 1.49 | 1.69 | 3.48 | 41.35 | 45.80 | 460.75 | neg | 7.20E−04 | 1.93E−02 |
| μs | | | | | | | | | |
| SP 600 μs | 1.30 | 1.48 | 2.79 | 35.99 | 43.47 | 376.08 | neg | 5.29E−04 | 1.08E−02 |
| CBP 600 | 1.23 | 1.42 | 2.59 | 35.16 | 44.43 | 632.29 | neg | 3.38E−04 | 7.59E−03 |
| μs | | | | | | | | | |
| SP 1000 | 0.98 | 1.12 | 1.98 | 27.26 | 40.50 | 287.89 | neg | 1.48E−04 | 3.39E−03 |
| μs | | | | | | | | | |
| CBP 1000 | 1.12 | 1.30 | 2.36 | 32.46 | 41.78 | 339.30 | neg | 1.48E−04 | 4.06E−03 |
| μs | | | | | | | | | |
| SP 2000 | 0.88 | 0.98 | 1.88 | 23.54 | 39.92 | 287.88 | neg | 1.48E−04 | 1.77E−03 |
| μs | | | | | | | | | |
| CBP 2000 | 1.01 | 1.17 | 2.11 | 29.22 | 40.75 | 341.66 | neg | 1.48E−04 | 1.96E−03 |
| μs | | | | | | | | | |
| ST1 | 1.26 | 1.45 | 1.34 | 35.70 | 179.75 | 273.41 | neg | 2.43E−04 | 6.06E−03 |
| ST2 | 1.63 | 1.87 | 1.79 | 45.40 | 162.89 | 288.88 | neg | 6.25E−04 | 1.42E−02 |
| SW500 | 0.99 | 1.12 | 1.08 | 27.88 | 161.31 | 191.20 | neg | 2.43E−04 | 7.11E−03 |
| SW5000 | 1.78 | 1.98 | 2.00 | 47.15 | 104.00 | 229.60 | neg | 4.25E−03 | 1.16E−01 |
| TP 1 | 2.90 | 3.23 | 2.71 | 79.44 | 57.79 | 374.89 | neg | 5.39E−03 | 1.78E−01 |
| TP 2 | 2.15 | 2.42 | 2.62 | 59.30 | 51.04 | 436.42 | neg | 1.96E−03 | 5.76E−02 |
| TMS 1 | 2.24 | 2.51 | 3.22 | 61.82 | 71.13 | 424.60 | neg | 3.10E−03 | 9.44E−02 |
| TMS 2 | 2.52 | 2.80 | 3.14 | 68.85 | 77.50 | 357.23 | neg | 8.83E−03 | 3.02E−01 |
| TMS 3 | 2.26 | 2.56 | 2.64 | 61.85 | 133.14 | 402.99 | neg | 2.63E−03 | 6.10E−02 |

| | | | | F2. Peak | |
|---|---|---|---|---|---|
| | | | | Constrained | G2. Peak |
| | | | | Dipole | Monopole |
| | | | | Voltage to | Voltage to |
| | E2. Peak Value of | | | Reach | Reach |
| | Constrained Current | | | Threshold | Threshold |
| | Injected At Monopole | | | (V), as | (V), as |
| | to Reach Threshold | | | conductivity | conductivity |
| | (mA), as conductivity | | | approaches | approaches |
| | approaches zero for the | | | zero for the | zero for the |
| A2. Source | system | | | system | system |
| Input | Ex- | Ex- | | All | All |
| Waveform | vivo 1 | vivo 2 | In-vivo | Sets | Sets |
| SP 65 μs | neg | 2.25E−03 | 4.95E−02 | 9.38E−02 | 1.49E−01 |
| CBP 65 | neg | 6.35E−03 | 2.05E−01 | 2.16E−01 | 3.43E−01 |
| μs | | | | | |
| SP 300 μs | neg | 5.29E−04 | 1.08E−02 | 2.07E−02 | 3.30E−02 |
| CBP 300 | neg | 7.20E−04 | 1.93E−02 | 3.06E−02 | 4.85E−02 |
| μs | | | | | |
| SP 600 μs | neg | 5.29E−04 | 1.08E−02 | 2.07E−02 | 3.30E−02 |
| CBP 600 | neg | 3.38E−04 | 7.59E−03 | 1.40E−02 | 2.22E−02 |
| μs | | | | | |
| SP 1000 | neg | 1.48E−04 | 3.39E−03 | 6.78E−03 | 1.07E−02 |
| μs | | | | | |
| CBP 1000 | neg | 1.48E−04 | 4.06E−03 | 8.31E−03 | 1.32E−02 |
| μs | | | | | |
| SP 2000 | neg | 1.48E−04 | 1.77E−03 | 3.92E−03 | 6.16E−03 |
| μs | | | | | |
| CBP 2000 | neg | 1.48E−04 | 1.96E−03 | 4.49E−03 | 7.20E−03 |
| μs | | | | | |
| ST1 | neg | 2.43E−04 | 6.06E−03 | 1.21E−02 | 1.92E−02 |
| ST2 | neg | 6.25E−04 | 1.42E−02 | 2.74E−02 | 4.36E−02 |
| SW500 | neg | 2.43E−04 | 7.11E−03 | 1.15E−02 | 1.85E−02 |
| SW5000 | neg | 4.25E−03 | 1.16E−01 | 1.53E−01 | 2.44E−01 |
| TP 1 | neg | 5.39E−03 | 1.78E−01 | 1.82E−01 | 2.90E−01 |
| TP 2 | neg | 1.96E−03 | 5.76E−02 | 7.24E−02 | 1.15E−01 |
| TMS 1 | neg | 3.10E−03 | 9.44E−02 | 1.14E−01 | 1.80E−01 |
| TMS 2 | neg | 8.83E−03 | 3.02E−01 | 2.76E−01 | 4.39E−01 |
| TMS 3 | neg | 2.63E−03 | 6.10E−02 | 1.04E−01 | 1.66E−01 |

Across the 19 stimulation waveforms and sources tested (TMS and DBS (mono/dipole for current constrained inputs)), the in-vivo stimulation thresholds were significantly higher than their ex-vivo counterparts and demonstrated a significant impact of the capacitive mechanisms on initiating spiking activity at the neural membranes (see FIGS. 7-9 & Tables 3-6)).

We also compared individual ohmic and capacitive contributions to the cellular response as we analyzed the systems while theoretically allowing the individual impedance components to approach zero; the in-vivo based solutions were significantly affected by the capacitive contributions.

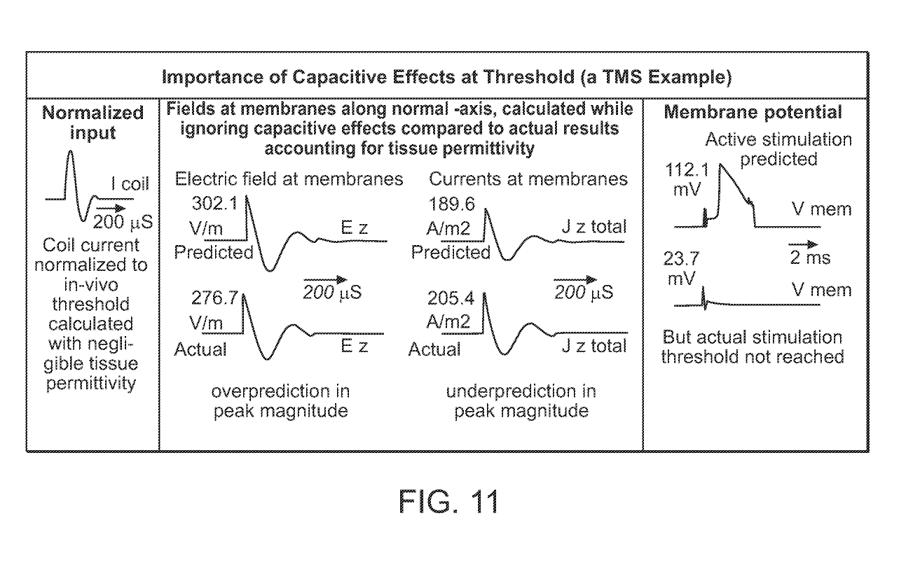

FIG. 11 is an example of simulation solutions based on artificially removing tissue capacitance compared to solutions including capacitive effects for a TMS example. Stimulation thresholds and membrane dynamics were analyzed for theoretical systems in which the tissue conductivity or permittivity was allowed to approach zero, for both the TMS and DBS systems (i.e., the stimulation fields were recalculated for all of the TMS and DBS models with the impedance properties set as such, and then the neural membrane response was analyzed). To test the importance of the capacitive field effects, we evaluated stimulation thresholds when the capacitive component was removed.

This figure shows an example of a predicted neural response to TMS stimulation when capacitive effects are ignored, as well as the actual response including capacitive effects (for the in-vivo based solutions). When the capacitive components were ignored the predicted fields and membrane potential response lead to an action potential, while the actual fields and resulting membrane response does not—such results can heavily impact dosing predictions. Note, the example herein is shown with near minimum field differences observed to highlight the importance on tissue capacitance on the neural response, more drastic responses are seen across many of the other 19 waveforms tested. This result was consistent across the current-constrained DBS (mono and dipole) solutions and for TMS solutions with neurons oriented perpendicular to the gray matter surface (see Table 6 for all 19 waveforms and impedance conditions tested)). The effect is less evident for TMS solutions for neurons oriented parallel to the gray matter surface, as those neurons oriented parallel to the surface are affected by fields approximately tangential to the coil interface, and tangential electric fields are continuous across the interlaying coil and tissue boundaries.

### Filtering Properties of Tissues:

Data herein show that the recorded impedance tissue values of the skin, skull, gray matter, and white matter substantially differed in magnitude and degree of frequency response from those in vitro values reported in the literature. Without being limited by any particular theory or mechanism, of action, it is believed that the differences between both in vitro and in vivo tissue impedance values reported in the literature result primarily from the degradation of cellular membranes and the termination of active processes that maintain the ionic concentrations in the intra and extracellular media. Such changes would theoretically lead to a decrease in tissue permittivity due to the loss of ionic double layers around the cellular bodies and an initial increase of conductivity due to changing extracellular ion concentrations, followed by a long-term permanent conductivity attenuation. On a macroscopic level, gross changes in structure caused by excision would be expected to translate into further deviations from living tissue impedances.

Regardless of the mechanism, data herein show that the use of tissue impedances derived from excised or damaged in vivo tissues does not adequately address the tissue-field response in a healthy in vivo system. Living tissue carries currents through both capacitive mechanisms and ohmic mechanisms. This is contrary to past theory that ionic mechanisms are the sole mechanism carrying neurostimulation currents, but in agreement with alpha dispersion theory predictions that stimulation currents are carried through both dipole polarizations and ionic conduction. Similarly, living tissue also has a frequency dependent impedance response to applied electromagnetic fields, making the brain tissue an effective filter, which is routinely considered negligible in brain stimulation applications. These fundamental tissue properties can have profound effects on the stimulatory fields and the neural effect.

### Tissue Effects on Fields:

To demonstrate the impact of the in vivo tissue impedances on the electromagnetics of brain stimulation, TMS and DBS generated field distributions were compared based on the measured in vivo impedance values and in vitro values drawn from the literature. There were consistent alterations of the field distributions as a function of the in vivo tissue impedance properties, which impacted the current density and electric field waveform dynamics, field amplitudes, vector field behavior, field penetrations, areas of maximum field distribution, and current composition in a time dependent and source dependent manner.

Although the in vivo tissue impedance effects impact every electrical neurostimulation technique, they maximally impact the stimulation fields when sources are located external to the targeted tissue, because the fields are impacted by not just the adjacent tissues, but by all of the tissues in between the stimulation source and the targeted tissue. This drastically impacts dosing related predictions of targeting, focality, and/or waveform dynamics made with noninvasive technologies presently used in the clinic. For instance, DBS demonstrated comparable field spreads and decreased electric field magnitudes but TMS demonstrated increased field spreads and decreased electric field magnitudes for the cortical in vitro and in vivo cases studied. Thus, DBS volumes of activation (VOA) would be overestimated with in vitro guided predictions.

Furthermore, stimulation techniques that drive fields across multiple boundaries demonstrate increasingly complicated temporal dynamics based on the unique tissue boundary conditions that constrain their behavior. For instance, when one examines the TMS field behavior for vectors approximately tangential and normal to the coil face-tissue boundaries (composite and z-field components respectively in FIGS. 7 and 8), the waveforms had distinct directional dependent temporal dynamics resulting from the tissue filtering across multiple boundaries, specifically due to the continuity of tangential electric fields and normal current densities across material boundaries. Such effects had the greatest impact along the brain surface, where stimulation was maximum for present noninvasive stimulators, and amplified at tissue heterogeneities (for example at gyri/sulci convolutions). However in many DBS implementations, where the stimulatory fields are confined to a single tissue, the boundary effects at the electrode tissue interface will have the most significant effect on the fields, as addressed with technologies already explored in the clinic. Although still advancing, clinical technologies used to predict dose-related stimulation metrics are more adequately addressed for invasive technologies like DBS, while alternate noninvasive stimulation technologies often misrepresent dosing-metrics (which could ultimately result in significant side-effects to a patient.

Importantly, tissue filtering has an impact on all systems implementing stimulation waveforms with specific temporal dynamics tailored to an individual neural structure. Tissues form a filtering network of capacitive and resistive elements, neither of which can be ignored, as currents in the tissues are carried through both mechanisms and the fields constrained by both tissue properties. These tissue effects are imperative to consider while evaluating the neural response to the electromagnetic fields and while developing ‘electromagnetic-dosing’ standards for neuro stimulation.

### Neural Response:

Predicted stimulation thresholds were consistently higher for the in vivo systems due to the attenuated electric fields (due to increased tissue impedance) and the altered waveform dynamics (due to tissue filtering). Importantly, opposite to conventional theory, the in vivo measurements of field-tissue interactions and the subsequent tissue based neural models of stimulation suggest that stimulation was driven by both dipole and free ion mechanisms in the tissues (capacitive and ohmic effects); the ratio of which is dependent on the time course of the stimulation waveforms, source type, and the relative directionality of the field-to-neuron under investigation.

Data herein present guidance for incorporating frequency dependent macroscopic tissue filtering effects with microscopic membrane potential models (e.g. the Hodgkin and Huxley model) to predict frequency dependent neural responses to external stimulation. This can be accomplished where tissue impedance recordings are coupled with loaded probe field measurements during simultaneous cellular patch recordings across the low frequency spectrum of stimulation. During the procedure, tissue impedances could be artificially altered through metabolic and chemical means to ascertain the neural effect (or to control the neural effect). Such results coupled with the analyses implemented herein could be used to develop a fundamental understanding of the microscopic interactions between the fields and cells during stimulation as driven by macroscopic predictions.

### Safety and Dosing Implications:

These results have a clear implication on safety and dosing considerations for neurostimulation. First, stimulation induced histological injury has classically been explored in terms of a stimulating waveforms' total current density amplitudes, charge per phase, and total charge. However both dipole and free charge effects (i.e., displacement and ohmic currents) are present during stimulation, and this offers a method to further characterize such processes as electrochemical reactions, heating, and electroporation, which can be linked to tissue injury.

Second, for TMS, stimulation is often deemed safe for patients if they meet MRI inclusion criteria. However, the slew rates and frequency range in which MRI and TMS techniques operate are different and the expected tissue responses between the two are not comparable. Thus, inclusion criteria for TMS patients, based on MRI compatibility, needs to be reevaluated based on the different spectral responses of the brain tissues to the different methods. Third, even greater care needs to be taken during stimulation of patients with pathological brain tissue (stroke, tumor, etc). From these measurements it is clear the stimulatory fields are highly dependent on the tissue effects, and we have little to no data on the impedances of tissue pathologies, so clinicians need to carefully manage the risk of such stimulation procedures against potential patient benefit. Finally in terms of dosing, data herein show tissue effects on stimulation targeting, expected magnitude of response, and directionality of effect.

### Example 2

### Electromagnetic Field Solutions

An example of the electromagnetic field solutions for predictive, guidance or optimization purposes are assessed as follows for typical electromagnetic energy based brain stimulation methods. First one must determine the type of analysis that is appropriate for the solution, if a quasistatics approach is appropriate it would be used for efficiency purposes, however a full electrodynamics solution can still be analyzed (and would be used always where quasistatics are not appropriate). Herein we develop quasistatics based solutions.

### Determining Validity of Quasistatic Electromagnetic Assumption:

In biological systems at low frequencies, electromagnetic systems are usually analyzed via quasistatic forms of Maxwell's equations. Quasistatic approximations are often made when the time rates of change of the dynamic components of the system are slow compared to the processes under study, such that the wave nature of the fields can be neglected. In practice, the electromagnetics of low frequency systems are generally addressed via either electroquasitatic (EQS) or magnetoquasistatic (MQS) methods where either the electric or magnetic fields are the primary fields of importance. In order to determine the appropriate solution method (and to justify the validity of the quasistatics for solving neurostimulation systems), one needs to address the source(s) (frequency/time dynamics and type) and the region of interest (materials and dimensions).

### Maxwell's Equations:

All electromagnetics begins with Maxwell's equations:

$$\begin{matrix}{Faraday}^{′} s Law \\ ∇ \times \vec{E} ( \vec{r} , t ) = - \frac{\partial }{\partial t} ( \vec{B} ( \vec{r} , t ) ) & ( 1 ) \\ {Ampere}^{′} s Law \\ ∇ \times \vec{H} ( \vec{r} , t ) = \frac{\partial }{\partial t} \vec{D} ( \vec{r} , t ) + {\vec{J}}_{f} ( \vec{r} , t ) & ( 2 ) \\ {Gauss}^{′} Law \\ ∇ \cdot \vec{D} ( \vec{r} , t ) = {p}_{f} ( \vec{r} , t ) & ( 3 ) \\ {Gauss}^{′} Magnetic Law \\ ∇ \cdot \vec{B} ( \vec{r} , t ) = 0 & ( 4 ) \\ Charge Conservation \\ ∇ \cdot {\vec{J}}_{f} ( \vec{r} , t ) = - \frac{\partial {p}_{f} ( \vec{r} , t )}{\partial t} & ( 5 )\end{matrix}$$

where \vec{E} is the electric field (V/m), \vec{H} is the magnetic field (A/m), \vec{D} is the displacement field (C/m²), \vec{B} is the magnetic flux density (T), \vec{J}_{f} is the free current density (A/m²), and p_{f} is the free charge density (C/m³); note, moving systems are ignored in this simplified analysis.
The electric displacement and magnetic flux density can be defined as:

\vec{D}(\vec{r},t)=∈₀\vec{E}(\vec{r},t)+\vec{P}(\vec{r},t)=∈\vec{E}(\vec{r},t) (6a)

\vec{B}(\vec{r},t)=μ₀\vec{H}(\vec{r},t)+\vec{M}(\vec{r},t)=μ\vec{H}(\vec{r},t) (6b)

where ∈ₒ (8.854×10 e−12 F/m) and μₒ (4π×10 e−7 H/m) are the permittivity and permeability of free space,), \vec{P} is the polarization density (C/m²), \vec{M} is the magnetization density (A/m), and ∈ and μ are the permittivity and permeability of the material under study.

In biological material, the free charge current density, \vec{J}_{f}, can be derived from analyzing the molar flux of ions in the system. For the analysis herein (i.e., in motion free macroscopic tissues with system dimensions greater than that of a Debye length where drift will dominate diffusion), the free charge current density can be shown to be equal to:

\vec{J}_{f}(\vec{r},t)=σ(\vec{r},t)\vec{E}(\vec{r},t) (7)

Where σ is the conductivity (S/m) of the material (free charge current density is also often also referred to as resistive, conductive, or ohmic current density—herein, we use the terms simultaneously in the main body of our article).

The total current density in the system is expressed by Ampere's Law, Eq (2), and is the sum of the ohmic and displacement (capacitive) current components (in most previous E & M neurostimulation developments, the capacitive elements are normally considered negligible, but they are not considered as such a priori in this development).

### Sinusoidal Steady State Analysis:

Maxwell's equations can also be presented in the frequency domain, where the fields are represented as time harmonic fields with an angular frequency w (i.e., assuming sinusoidal steady state solutions for individual frequencies). This could also be used as the basis for any computational software/method that would be used to guide a solution method. Using the following phasor notation:

$$\overline{\vec{E}} ( \vec{r} , t ) = Re \{ \overline{\vec{E} ( \vec{r} )} {e}^{j ω t} \}$$

Maxwell's equations can be represented as:

$$\begin{matrix}Faraday ' s Law ∇ \times \overline{\vec{E} ( \vec{r} )} = - jω ( \overline{\vec{B} ( \vec{r} )} ) = - jω ( μ \overline{H ( \vec{r} )} ) & ( 1 b ) \\ \begin{matrix}Ampere ' s Law ∇ \times \overline{\vec{H} ( \vec{r} )} = jω \overline{\vec{D} ( \vec{r} )} + \overline{{\vec{J}}_{f} ( \vec{r} )} \\ = jωɛ \overline{\vec{E} ( \vec{r} )} + σ \overline{\vec{E} ( \vec{r} )}\end{matrix} & ( 2 b ) \\ Gauss ' Law ∇ \cdot \overline{\vec{D} ( \vec{r} )} = \overline{{p}_{f} ( \vec{r} )} & ( 3 b ) \\ Gauss ' Magnetic Law ∇ \cdot \overline{\vec{B} ( \vec{r} )} = 0 & ( 4 b ) \\ Charge Conservation ∇ \cdot \overline{{\vec{J}}_{f} ( \vec{r} )} = - j ω \overline{{p}_{f} ( \vec{r} )} & ( 5 b )\end{matrix}$$

And it should be noted that while data herein are based on solutions assuming sinusoidal steady state solutions, any arbitrary time domain system can be solved for, with sinusoidal steady state analysis via Fourier theory (see below).

### Normalized Equations:

Next in order to develop and justify the EQS and MQS equations, equations (1b)-(5b) are normalized where: coordinates are normalized to a typical length constant, l; the angular frequency normalized to a typical source value, ω_{typ}, which is equal to 2π*f (the field frequency); the material constants normalized to typical values, σ_{typ}, ∈_{typ}, μ_{typ} corresponding to those of the tissue being analyzed at the field frequency under study; and, the inverse of the typical angular frequency is referred to as the characteristic time, τ.

This leads to the below such that:

$$( x , y , z ) = ( l \underline{x} , l \underline{y} , l \underline{z} ) , ∇ = \frac{\underline{∇}}{l} , σ = {σ}_{typ} \underline{σ} , ɛ = {ɛ}_{typ} \underline{ɛ} , \\ μ = {μ}_{typ} \underline{μ} , ω = {ω}_{typ} \underline{ω} , {ω}_{typ}^{- 1} = τ \qquad ( 8 )$$

where the underbars indicate normalized values.
To develop the EQS and MQS systems, Maxwell's equations are normalized following two different paths, which are presented in parallel. The first normalization is developed relative to a characteristic electric field, Eₒ, for the EQS derivation and the second normalization to a characteristic magnetic field, H_{O}, for the MQS derivation. Note, in what follows the (\vec{r}) notation is omitted except where ambiguity is possible, as all the complex field quantities as provided are functions of \vec{r} only as written.

First, the fields are normalized to typical length, material, and time values seen in the systems under study:

$$\begin{matrix}EQS \\ {E}^{\overline{\to }} = {E}_{o} {\underline{E}}^{\overline{\to }} , \overline{{p}_{f}} = \frac{{ɛ}_{typ} {E}_{o}}{l} \overline{\underline{{p}_{f}}} \\ \vec{H} = \frac{{ɛ}_{typ} {E}_{o} l}{τ} {\underline{H}}^{\overline{\to }}\end{matrix} \vert \begin{matrix}\begin{matrix}MQS \\ {H}^{\overline{\to }} = {H}_{o} {\underline{H}}^{\overline{\to }} , \overline{{p}_{f}} = \frac{{ɛ}_{typ} {μ}_{typ} {H}_{o}}{τ} \overline{\underline{{p}_{f}}}\end{matrix} \\ {E}^{\overline{\to }} = \frac{{μ}_{typ} {H}_{o} l}{τ} {\underline{E}}^{\overline{\to }}\end{matrix} \qquad ( 9 )$$

Next (8)-(9) can be inserted into (1b)-(5b) to develop:

$$\begin{matrix}\begin{matrix}\begin{matrix}\begin{matrix}∇ \times {\underline{E}}^{\overline{\to }} = - j ( \frac{{μ}_{typ} {ɛ}_{typ} {l}^{2}}{{τ}^{2}} ) \underline{ω} \underline{μ} ( {\underline{H}}^{\overline{\to }} ) \\ \begin{matrix}\underline{∇} \times {\underline{H}}^{\overline{\to }} = j \underline{ω} \underline{ɛ} ( \overline{\underline{\vec{E}}} ) + \\ τ ( \frac{{σ}_{typ}}{{ɛ}_{typ}} ) \underline{σ} \overline{\underline{\vec{E}}}\end{matrix}\end{matrix} \\ \underline{∇} \cdot \underline{ɛ} {\underline{E}}^{\overline{\to }} = \overline{{\underline{p}}_{f}}\end{matrix} \\ \underline{∇} \cdot ( \underline{μ} {\underline{H}}^{\overline{\to }} ) = 0\end{matrix} \\ \underline{∇} \cdot \underline{σ} {\underline{E}}^{\overline{\to }} = - {j ( τ \frac{{σ}_{typ}}{{ɛ}_{typ}} )}^{- 1} \underline{ω} \overline{\underline{{p}_{f}}}\end{matrix} \vert \begin{matrix}\begin{matrix}\begin{matrix}\underline{∇} \times {\underline{E}}^{\overline{\to }} = - j \underline{ω} \underline{μ} ( {\underline{H}}^{\overline{\to }} ) \\ \begin{matrix}\underline{∇} \times {\underline{H}}^{\overline{\to }} = j ( \frac{{μ}_{typ} {ɛ}_{typ} {l}^{2}}{{τ}^{2}} ) \underline{ω} \underline{ɛ} \overline{\underline{\vec{E}}} + \\ ( \frac{{μ}_{typ} {ɛ}_{typ} {l}^{2}}{{τ}^{2}} ) \underline{σ} \overline{\underline{\vec{E}}} \\ \underline{∇} \cdot \underline{ɛ} {\underline{E}}^{\overline{\to }} = \overline{{\underline{p}}_{f}}\end{matrix}\end{matrix} \\ \underline{∇} \cdot ( \underline{μ} {\underline{H}}^{\overline{\to }} ) = 0\end{matrix} \\ \underline{∇} \cdot \underline{σ} {\underline{E}}^{\overline{\to }} = - {j ( τ \frac{{σ}_{typ}}{{ɛ}_{typ}} )}^{- 1} \underline{ω} \overline{\underline{{p}_{f}}}\end{matrix} \qquad \begin{matrix}( 1 c ) \\ \\ ( 2 c ) \\ \\ \\ \\ ( 3 c ) \\ \\ ( 4 c ) \\ \begin{matrix} \\ ( 5 c )\end{matrix}\end{matrix}$$

which can be rewritten as follows:

$$\begin{matrix}\begin{matrix}\begin{matrix}\begin{matrix}\underline{∇} \times {\underline{E}}^{\overline{\to }} = - j β ( \underline{ω μ {H}^{\overline{\to }}} ) \\ \underline{∇} \times {\underline{H}}^{\overline{\to }} = j \underline{ω} \underline{ɛ} ( {\underline{E}}^{\overline{\to }} ) + ( \frac{τ}{{τ}_{e}} ) \underline{σ} \overline{\underline{\vec{E}}}\end{matrix} \\ \underline{∇} \cdot \underline{ɛ} {\underline{E}}^{\overline{\to }} = \overline{{\underline{p}}_{f}}\end{matrix} \\ \underline{∇} \cdot ( \underline{μ} {\underline{H}}^{\overline{\to }} ) = 0\end{matrix} \\ \underline{∇} \cdot \underline{σ} {\underline{E}}^{\overline{\to }} = - {j ( \frac{τ}{{τ}_{e}} )}^{- 1} \underline{ω} \overline{\underline{{p}_{f}}}\end{matrix} \vert \begin{matrix}\begin{matrix}\begin{matrix}\begin{matrix}\underline{∇} \times {\underline{E}}^{\overline{\to }} = - j \underline{ω} \underline{μ} ( {\underline{H}}^{\overline{\to }} ) \\ \underline{∇} \times {\underline{H}}^{\overline{\to }} = jβ \underline{ω} \underline{ɛ} {\underline{E}}^{\overline{\to }} + ( \frac{{τ}_{m}}{τ} ) \underline{σ} \overline{\underline{\vec{E}}}\end{matrix} \\ \underline{∇} \cdot \underline{ɛ} {\underline{E}}^{\overline{\to }} = \overline{{\underline{p}}_{f}}\end{matrix} \\ ∇ \cdot ( \underline{μ} {\underline{H}}^{\overline{\to }} ) = 0\end{matrix} \\ \underline{∇} \cdot \underline{σ} {\underline{E}}^{\overline{\to }} = - j {β ( \frac{{τ}_{m}}{τ} )}^{- 1} \underline{ω} \overline{\underline{{p}_{f}}}\end{matrix} \qquad \begin{matrix}\begin{matrix}\begin{matrix}\begin{matrix}\begin{matrix}\begin{matrix}\begin{matrix}\begin{matrix}( 1 d ) \\ \end{matrix} \\ ( 2 d )\end{matrix} \\ \end{matrix} \\ ( 3 d )\end{matrix} \\ \end{matrix} \\ ( 4 d )\end{matrix} \\ \end{matrix} \\ ( 5 d )\end{matrix}$$

### Where

$${τ}_{e} = \frac{{ɛ}_{typ}}{{σ}_{typ}} ,$$

the charge relaxation time; σ_{typ}μ_{typ}l²=τₘ, the magnetic diffusion time; and

$$β = \frac{{μ}_{typ} {ɛ}_{typ} {l}^{2}}{{τ}^{2}} .$$

Note τₑₘ is equal to l/c, the time for the speed of light to cross a typical length, l, which is equal to the product of the charge relaxation time and the magnetic diffusion time.

### Quasistatic Equations and Justification:

For typical values and lengths used in this problem (modeling systems analyzed during brain stimulation), one can develop the different time constants corresponding to the different impedance sets, from Example 1 above, as analyzed in the Examples below or could be used as a basis for assessing computational methods implemented in this disclosure. As an example below, typical constants are tabulated for the three different impedance sets that have been analyzed for a prototypical region of gray matter, assuming a 500 Hz source and a 0.2 cm tissue thickness in the prototypical region of interest. See Table 7 below.

TABLE 7
Typical values corresponding to 500 Hz source for gray matter

| | Typical | Typical | Typical | Typical | Angular Frequency | Typical |
|---|---|---|---|---|---|---|
| Typical Values | Permittivity (F/m) | Conductivity (S/m) | Permeability (H/m) | Length (m) | (rad/sec) | Time (s) |
| Measured | 5.469E−5 | 1.413E−1 | 1.25664E−06 | 0.002 | 3141.592654 | 3.183E−4 |
| Brooks | 2.806E−6 | 9.636E−2 | 1.25664E−06 | 0.002 | 3141.592654 | 3.183E−4 |
| Common | 1.062E−9 | 2.760E−1 | 1.25664E−06 | 0.002 | 3141.592654 | 3.183E−4 |

| Constants | τ_{e (s)} | τ_{m (s)} | τ_{em (s)} | τ₍ₛ₎ | β |
|---|---|---|---|---|---|
| Measured | 3.870E−4 | 7.104E−13 | 3.316E−11 | 3.183E−4 | 1.08535E−14 |
| Brooks | 2.912E−5 | 4.843E−13 | 7.511E−12 | 3.183E−4 | 5.56755E−16 |
| Common | 3.850E−9 | 1.387E−12 | 1.462E−13 | 3.183E−4 | 2.10839E−19 |

Given the values tabulated above, where the β values are much less than unity, either EQS or MQS approximations could be justified; however, EQS is suggested, as τₑ>τₘ for all of the impedance sets under study (note, the above table can be expanded for all the tissues, impedance sets, and full frequency band under study to justify the use of quasistatics). Formally one could expand the normalized functions around β, where the zero order equations (1)-(5) are simply:

$$\begin{matrix}\begin{matrix}\begin{matrix}\begin{matrix}EQS \\ \underline{∇} \times \overline{\underline{\vec{E}}} = 0 \\ \underline{∇} \times {\underline{H}}^{\overline{\to }} = - j \underline{ω} \underline{ɛ} ( \overline{\underline{\vec{E}}} ) + ( \frac{τ}{{τ}_{e}} ) \underline{σ} \overline{\underline{\vec{E}}}\end{matrix} \\ \underline{∇} \cdot \underline{ɛ} {\underline{E}}^{\overline{\to }} = {\underline{p}}_{f}\end{matrix} \\ \underline{∇} \cdot ( \underline{μ} {\underline{H}}^{\overline{\to }} ) = 0\end{matrix} \\ \underline{∇} \cdot \underline{σ} {\underline{E}}^{\overline{\to }} = - {j ( \frac{τ}{{τ}_{e}} )}^{- 1} \underline{ω} \overline{\underline{{p}_{f}}}\end{matrix} \vert \begin{matrix}\begin{matrix}\begin{matrix}\begin{matrix}MQS \\ \underline{∇} \times \overline{\underline{\vec{E}}} = j \underline{ω} \underline{μ} ( {\underline{H}}^{\overline{\to }} )\end{matrix} \\ \underline{∇} \times {\underline{H}}^{\overline{\to }} = ( \frac{{τ}_{m}}{τ} ) \underline{σ} \overline{\underline{\vec{E}}} \\ \underline{∇} \cdot \underline{ɛ} {\underline{E}}^{\overline{\to }} = {\underline{p}}_{f}\end{matrix} \\ \underline{∇} \cdot ( \underline{μ} {\underline{H}}^{\overline{\to }} ) = 0\end{matrix} \\ \underline{∇} \cdot \underline{σ} \overline{\underline{\vec{E}}} = 0\end{matrix} \qquad \begin{matrix}\begin{matrix}\begin{matrix}\begin{matrix}\begin{matrix}\begin{matrix} \\ ( 1 e )\end{matrix} \\ \end{matrix} \\ ( 2 e )\end{matrix} \\ \end{matrix} \\ ( 3 e )\end{matrix} \\ \begin{matrix}\begin{matrix} \\ ( 4 e )\end{matrix} \\ ( 5 e )\end{matrix}\end{matrix}$$

which are now rewritten with their normalizations removed:

$$\begin{matrix}\begin{matrix}∇ \times \overline{\vec{E}} = 0 & ∇ \times \overline{\vec{E}} = - jω ( μ \overline{H} )\end{matrix} & ( 1 f ) \\ \begin{matrix}∇ \times \overline{\vec{H}} = - jωɛ \overline{\vec{E}} + σ \overline{\vec{E}} & ∇ \times \overline{\vec{H}} = σ \overline{\vec{E}}\end{matrix} & ( 2 f ) \\ \begin{matrix}∇ \cdot ɛ \overline{\vec{E}} = \overline{{p}_{f}} & ∇ \cdot ɛ \overline{\vec{E}} = \overline{{p}_{f}}\end{matrix} & ( 3 f ) \\ \begin{matrix}∇ \cdot ( μ \overline{\vec{H}} ) = 0 & ∇ \cdot ( μ \overline{\vec{H}} ) = 0\end{matrix} & ( 4 f ) \\ \begin{matrix}∇ \cdot σ \overline{\vec{E}} = - jω \overline{{p}_{f}} & ∇ \cdot σ \overline{\vec{E}} = 0\end{matrix} & ( 5 f )\end{matrix}$$

and are finally reordered in the order they are typically pursued in practice, and presented in the final sinusoidal steady state EQS and MQS forms:

$$\begin{matrix}\begin{matrix}{∇ \times \overline{\vec{E}} = 0}^{EQS} & {∇ \times \overline{\vec{H}} = σ \overline{\vec{E}}}^{MQS}\end{matrix} & \begin{matrix} \\ ( EQS 1 , MQS 1 )\end{matrix} \\ \begin{matrix}∇ \cdot ɛ \overline{\vec{E}} = \overline{{p}_{f}} & ∇ \cdot ( μ \overline{\vec{H}} ) = 0\end{matrix} & ( EQS2 , MQS2 ) \\ \begin{matrix}∇ \cdot σ \overline{\vec{E}} = - jω \overline{{p}_{f}} & ∇ \times \overline{\vec{E}} = - jω ( μ \overline{H} )\end{matrix} & ( EQS3 , MQS3 ) \\ \begin{matrix}∇ \times \overline{\vec{H}} = jω ɛ \overline{\vec{E}} + σ \overline{\vec{E}} & ∇ \cdot σ \overline{\vec{E}} = 0\end{matrix} & ( EQS4 , MQS4 ) \\ \begin{matrix}∇ \cdot ( μ \overline{\vec{H}} ) = 0 & ∇ \cdot ɛ \overline{\vec{E}} = \overline{{p}_{f}}\end{matrix} & ( EQS5 , MQS5 )\end{matrix}$$

Additionally one needs to define boundary conditions for the systems which contain multiple tissues, where between a material₁ and material₂:

$$\begin{matrix}\begin{matrix} n \cdot ( {ɛ}_{1} \overline{{\vec{E}}_{1}} - {ɛ}_{2} \overline{{\vec{E}}_{2}} ) = {\overline{σ}}_{s} & n \cdot ( {μ}_{1} \overline{{\vec{H}}_{1}} - {μ}_{2} \overline{{\vec{H}}_{2}} ) = 0\end{matrix} & ( BC1 ) \\ \begin{matrix} n \times ( \overline{{\vec{E}}_{1}} - \overline{{\vec{E}}_{2}} ) = 0 & n \times ( \overline{\vec{{H}_{1}}} - \overline{\vec{{H}_{2}}} ) = \overline{\vec{{K}_{S}}}\end{matrix} & ( BC2 ) \\ \begin{matrix}n \cdot ( {σ}_{1} \overline{\vec{{E}_{1}}} - {σ}_{2} \overline{\vec{{E}_{2}}} ) + ∇ \cdot \overline{\vec{{K}_{S}}} = - jω \overline{{σ}_{s}} & n \cdot ( {σ}_{1} \overline{{\vec{E}}_{1}} - {σ}_{2} \overline{{\vec{E}}_{2}} ) + ∇ \cdot \overline{\vec{{K}_{S}}} = 0\end{matrix} & ( BC3 )\end{matrix}$$

where σₛ is the surface charge density (coul/m²) accumulating between the regions, and Kₛ defines a surface current (amp/m) between the regions. Furthermore, biological tissues typically demonstrate the permeability of free space in the absence of artificial magnetic materials (such as injected ferrofluids), thus an assumption is made throughout the rest of this development that the materials maintain the permeability of free space. Additionally, the free charge density, in EQS2 and MQS5, is generally considered equal to zero for macroscopic tissues due to bulk charge electroneutrality justifications (i.e., for systems where the charge relaxation times of the tissues are shorter than the times characterizing the systems under study and in regions more than a few Debye lengths in distance away from tissue boundaries, and/or for systems with uniform conductivity and permittivity (even for non quasistatic systems) in regions more than a few Debye lengths in distance away from tissue boundaries (i.e., locations of surface charge)).

These above equations represent EQS1-5, MQS1-5 and the corresponding boundary equations (BC 1-3) represent the starting point for the computational determination of the electromagnetic field distributions and dosing in the tissues to be stimulated. Here we represent the equations in the SSS, but they can conversely be presented in the time domain (and solved in the time domain). Below we demonstrate how to apply the above equations to both the electrical and magnetic sources, with analytical solutions.

### Analytical Electrical Source Based Solutions:

Assuming a particular voltage or current input waveform at the electrode source interface, given EQS1, ∇× [symbol] =0, one can define a scalar potential Φ such that:

$$Δ \overline{Φ} = - \overline{\vec{E}} \qquad DBS1$$

Second EQS 2 and EQS 3 can combined to yield:

$$j ω ∇ \cdot ɛ \overline{\vec{E}} + ∇ \cdot σ \overline{\vec{E}} = 0 \qquad DBS2$$

(note the same could be shown by taking the divergence of Ampere's Law, EQS4). Finally DBS 1 and DBS2 can be combined to yield:

∇·(jω∈∇ Φ̅+σ∇ Φ̅)=0 DBS 3

DBS 3 can be solved with standard boundary value methods given a defined source, system geometry, and material properties of the system under study. Often, electrical systems are analyzed from a current source view-point, where we could introduce a volume distribution of current sources, such that:

$$∇ \cdot {J}^{\overline{\to }} = \overline{{I}_{s}} ⇔ {∮}_{S} {J}^{\overline{\to }} \cdot \partial a = {\int }_{v} \overline{{I}_{s}} d v \qquad DBS4$$

where Īₛ defines a source distribution of current source singularities (A/m³), such that:

∇·(jω∈+σ)∇ Φ̅= I̅ₛ DBS 5

DBS 5 is the typical starting point for many electrical problems. With a simple point source, l̅, in a single isotropic, homogenous tissue, DBS 5 can be solved as:

$$\overline{Φ} = \frac{\overline{I}}{4 π ( j ω ɛ + σ ) r} \qquad DBS6$$

where r is the distance between the electrode source and the neuron being evaluated. Further, with a distribution of current source singularities, the total voltage can be determined with the superposition principle.

### Analytical Magnetic Based Solutions:

The starting point is the EQS system of equations (EQS1-5), given that the time constants above, τₑ>τₘ. Assuming a magnetic coil source, driven by a particular current waveform, it is noted at the onset that the curl of the electric field cannot be equal to zero, ∇× [symbol] ≠0, as is typical of EQS1, because a magnetic source magnetic field, [symbol] , can drive a non-curl free induced electric field in the system. Given this, the electric field, [symbol] , is not defined via a scalar electric potential as above.

Additionally, it is assumed that the conductivity and permittivity are uniform in the individual tissue layers and that the region of interest in each of the tissue layers describes behavior more than a few Debye lengths away from the tissue boundaries. These assumptions allow for the assumption that the free charge in the EQS 2 is equal to zero in the region of interest.

As such, the analytic problem becomes tractable by solving for the induced electrical field as a function of its homogenous and particular parts, based on EQS1 and EQS2:

$$\begin{matrix}\overline{\vec{E}} = \overline{{\vec{E}}_{h}} + \overline{{\vec{E}}_{p}} & TMS 1 \\ ∇ \times \overline{{\vec{E}}_{p}} = jω \overline{\vec{{μ}_{o} {H}_{s}}} , \\ ∇ \times \overline{{\vec{E}}_{h}} = 0 , \\ ∇ \cdot \overline{{\vec{E}}_{p}} = 0 , \\ ∇ \cdot \overline{{\vec{E}}_{h}} = 0 & TMS 2 a , 2 b , 3 a , 3 b\end{matrix}$$

and, note that the particular solution is forced by the magnetic source field.

Next, if one concentrates on the particular solution, and focuses on equation TMS 2a, Poisson's Equation can be developed for the particular solution of the electric field based on the magnetic source field as follows:

$$∇ \times ( ∇ \times \overline{{\vec{E}}_{p}} = jω \overline{\vec{{μ}_{o} {H}_{s}}} ) ⇒ ∇ ( ∇ \cdot \overline{{\vec{E}}_{p}} ) - {∇}^{2} \overline{{\vec{E}}_{p}} = ∇ \times jω \overline{\vec{{μ}_{o} {H}_{s}}} ⇒ {∇}^{2} \overline{{\vec{E}}_{p}} = - ∇ \times jω \overline{\vec{{μ}_{o} {H}_{s}}} \qquad TMS 4$$

which has the solution:

$$\overline{{\vec{E}}_{p} ( \vec{r} )} = - \frac{1}{4 π} {\int }_{{V}^{′}} \frac{j ω \overline{\vec{{μ}_{o} {H}_{s}}} ( \vec{{r}^{′}} ) \times {\hat{i}}_{{r}^{′} r}}{{\vert r - {r}^{′} \vert}^{2}} \partial {V}^{′} \qquad TMS5$$

where r′ is the coordinate of the magnetic field source [symbol] ; r is the coordinate at which [symbol] is evaluated (the observer coordinate); and î_{r′r} is the unit vector pointing from r′ to r. V′ defines the volume in which the source magnetic field, [symbol] , is found.

Given the fact that τ>>τₘ, the magnetic source field, [symbol] , can be determined solely based on characteristics of the magnetic source coil and its driving current, [symbol] . First one starts with the EQS 5, ∇·(μₒ [symbol] )=0 and defines a magnetic vector potential, ∇× [symbol] =μₒ [symbol] and sets the gauge such that ∇· [symbol] =0, so that:

$$∇ \times ( ∇ \times {{A}^{\overline{\to }}}_{s} = {μ}_{o} \overline{{\vec{H}}_{s}} ) ⇒ ∇ ( ∇ \cdot {{A}^{\overline{\to }}}_{s} ) - {∇}^{2} {{A}^{\overline{\to }}}_{s} = {μ}_{o} ( ∇ \times \overline{{\vec{H}}_{s}} ) ⇒ {∇}^{2} {{A}^{\overline{\to }}}_{s} = {μ}_{o} \overline{{\vec{J}}_{s}} ⇒ \overline{{\vec{H}}_{s} ( \vec{r} )} = \frac{1}{{μ}_{o}} ∇ \times ( \overline{{\vec{A}}_{s} ( \vec{r} )} = \frac{{μ}_{o}}{4 π} {\int }_{{V}^{′}} \frac{\overline{{\vec{J}}_{s}} ( {r}^{′} )}{{\vert r - {r}^{′} \vert}^{2}} d {V}^{′} ) ⇒ \overline{{\vec{H}}_{s} ( \vec{r} )} = - \frac{1}{4 π} {\int }_{{V}^{′}} \frac{{{J}_{s}}^{\overline{\to }} ( \vec{{r}^{′}} ) \times {\hat{i}}_{{r}^{′} r}}{{\vert r - {r}^{′} \vert}^{2}} d {V}^{′} \qquad TMS6$$

where r′ is the coordinate of the current source field, [symbol] ; r is the coordinate at which [symbol] is evaluated (the observer coordinate); î_{r′r} is the unit vector pointing from r′ to r; and V′ defines the volume in which the source magnetic field, [symbol] , is found. This is simply the Biot-Savart Law, and between TMS5 and TMS 6 one can solve for [symbol] .

The homogenous equations simply reduce to Laplace's equation. Since ∇× [symbol] =0, one can define a scalar potential Φₕ such that:

$$Δ \overline{{Φ}_{h}} = - \overline{{\vec{E}}_{h}} \qquad TMS 7$$

which can then be plugged into TMS 3b to get Laplace's equation:

$$∇ \cdot ɛ \overline{{\vec{E}}_{h}} = ∇ \cdot ɛ ∇ \overline{{Φ}_{h}} = ɛ ∇ \cdot ∇ {\overline{Φ}}_{h} + ∇ ɛ \cdot ∇ {\overline{Φ}}_{h} = {∇}^{2} \overline{{Φ}_{h}} = 0 \qquad TMS8$$

assuming that individual tissue permittivity is isotropic and uniform.
Given the particular and homogenous solutions of the electric field, [symbol] , the problem reduces to a boundary value problem that can be solved for a given source, system geometry, and material constants of the tissues under study.

These analytical equations can serve as the basis for the solutions to be determined in sinusoidal steady state, which can be completed with computational methods (such as those exemplified in example 1 or the detailed description of this document) when analytical solutions are not attainable, with the appropriate boundary conditions to be applied (as explained above).

The examples above are provided to develop solutions in the SSS, such as for example when a system reaches equilibrium with a sinusoidal source. This method could be used to develop energy field solutions in the tissues in the frequency domain, or complete time domain solutions. For determining solutions in the time domain with SSS methods one could first convert the time domain input waveforms of the source (i.e., the stimulation waveform source) into the frequency domain via discrete Fourier transforms in any computing environment. Second, the electromagnetic field responses of the individual frequency components of the stimulation source to the tissue to be stimulated could be analyzed in the sinusoidal steady state in increments, determined dependent on desired solution resolution, with separate sinusoidal steady state (SSS) computational models, such as finite element methods such as with the Ansoft Maxwell package that numerically solves the problem via a modified T-Ω method, based on the CAD renderings of the tissue(s) to be stimulated, such as could be developed with an MRI (where individual tissue components of the model are assigned tissue impedance parameters for the individual tissues based on the frequency components analyzed and source properties are included relative to the tissue being stimulated (e.g., the source position (relative to tissue to be stimulated,) orientation (relative to tissue to be stimulated), geometry, and materials). Finally, the individual SSS solutions could be combined and used to rebuild a solution in the time domain via inverse Fourier methods (e.g., transforming from the frequency back to the time domain as in Electromagnetic Fields and Energy by Hermann A. Haus and James R. Melcher (1989)). The transient electrical field and current density waveforms are then analyzed in terms of field magnitudes, orientations, focality, and penetration as a function of time and tissue impedance.

The field solutions could also be developed completely within the time domain. In practice analytical field solutions to the neural stimulation problems are not easily attainable given the complex tissue distribution/geometry, tissue electromagnetic properties, and source characteristics of the systems under study. Thus, numerical methods are pursued to determine the field distributions; see the main text for a discussion of the numerical methods used (or for example see (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007), (Wagner, Fregni, Fecteau, Grodzinsky, Zahn and Pascual-Leone, Transcranial direct current stimulation: a computer-based human model study, Neuroimage, 35, (3), 1113-24, 2007)). (In example 1 above we started with time domain source functions of the stimulating waveforms, I(t) and V(t) (corresponding to typical TMS coil currents and DBS electrode voltage and currents used in clinical practice), and transformed these waveforms into the frequency domain using a discrete Fourier transform (DFT). The derived frequency components served as the source inputs to MRI guided Sinusoidal Steady State(SSS) finite element method (FEM) electromagnetic field solvers (developed based on the head/brain geometry analyzed, and the individual tissue impedance sets analyzed); where the each individual frequency component solution was determined via a Matlab controlled Ansoft field solvers (TMS via a modified magnetic diffusion equation implementing a modified T-Ω method, and the DBS solutions via a modified Laplacian, see (Wagner et al., 2004; Wagner et al., 2007)). Finally, the solutions were rebuilt in the time domain via inverse Fourier transforms.)

The field models are then coupled with conductance-based compartmental models of brain stimulation, with the external driving field determined as above. Neuron (or cell) parameters are drawn from the targeted tissue. (In our example 1 above, membrane dynamics were solved using Euler's method. Neurostimulation thresholds were calculated by integrating the field solution with these compartmental models. For each stimulating waveform, source, and tissue property model an iterative search was performed to find the smallest constrained input that generated an action potential, analyze the membrane dynamics as a function of on flow, and with network models analyzed the integrated effects. Importantly, the simultaneous integration and solution of the neural response and stimulation field allowed for tuned responses, optimized responses, and maximal responses of the targeted tissues.)

The electromagnetic models can be combined with models of other energy types, such as chemical, mechanical, thermal, and/or optical energies. For instance one could use these methods to analyze the electrical, mechanical, and chemical processes ongoing in the tissues during stimulation (such as analyzing fluid flow, ionic movement (such as from electrical, chemical, and mechanical forces), and chemical reactions driven by the fields).

### Example 3

### EMS Field Modeling (Electromechanical Modeling)

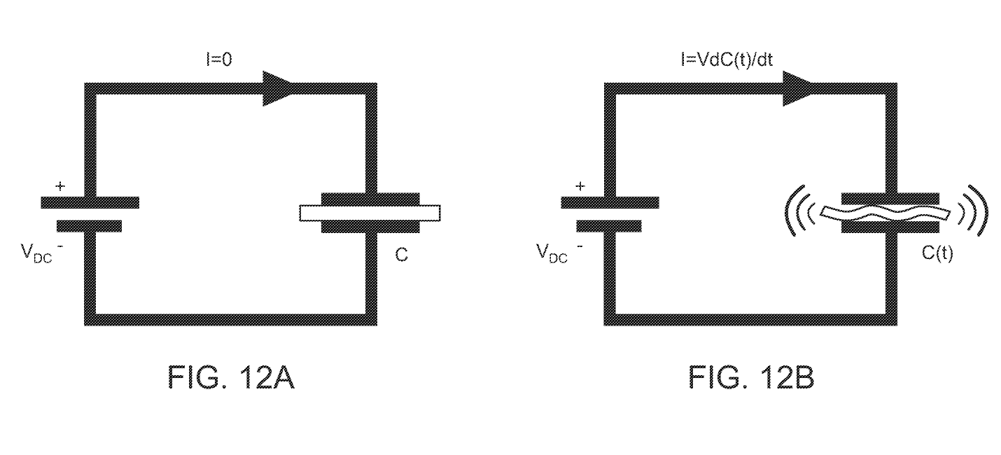

Electromechanical stimulation (EMS) implements combined electromagnetic and mechanical energy to stimulate neural tissues noninvasively (note EMS is also referred to as electromechanical throughout the document). During electromechanical stimulation, a displacement current is generated in a tissue by mechanically altering the tissue's permittivity characteristics relative to an applied sub-threshold electrical field such that the total current density in the region of displacement current generation is capable of altering neural activity, see FIG. 12 for a simplified circuit representation of how electromechanical energy can be combined, whereby mechanical energy can impact the electrical energy (In A, with a DC voltage source, the steady-state current in the in the capacitor is zero. However in B, a mechanical stimulus alters the capacitor's dielectric permittivity, and even with a DC source a new current is generated equal to VdC(t)/dt (where C=AE(t)/d, and A is the area of the plates, d the distance between, and ∈(t) is the dielectric permittivity as a function of time)). The displacement current densities generated during electromechanical stimulation can be quite significant, even with a low amplitude applied electromagnetic fields, because in the low frequency bands used for electromechanical stimulation, tissue permittivities are considerably elevated due to “alpha dispersion” effects (Hart, Toll, Berner and Bennett, The low frequency dielectric properties of octopus arm muscle measured in vivo, Phys. Med. Biol., 41, (2043-2052, 1996), (Foster and Schwan, Dielectric Properties of Tissues, Biological Effects of Electromagnetic Fields, 25-102, 1996), (Hart and Dunfree, In vivo measurements of low frequency dielectric spectra of a frog skeletal muscle, Phys. Med. Biol., 38, (1099-1112, 1993), (Dissado, A fractal interpertation of the dielectric response of animal tissues, Phys. Med. Biol., 35, (11), 1487-1503, 1990), (Martinsen, Grimmes and Schwan, Interface Phenomena and Dielectric Properties of Biological Tissue, Encyclopedia of Surface and Colloid Science, 2002), (Schwarz, J Phys Chem, 66, (2636, 1962), (Grosse, Permitivity of suspension of charged particles in electolyte solution, J. Chem. Phys., 91, (3073, 1987) and thus relatively minimum permittivity changes, in comparison to their overall permittivity magnitude, can still lead to a significant displacement currents (furthermore, just the relative change in permittivity (to its previous value before being altered) can lead to significant currents in the presence of the electric field).

Thus, by using current injection methods similar to tDCS (but with a lower amplitude source), broad cortical regions can be subjected to currents of insufficient magnitude to effect neural behavior, but by combining mechanical methods in focused regions altered currents can be generated to stimulate cells in the region. Thus, the technique allows a method to amplify, focus, alter the direction of, and/or attenuate currents in living tissue without the limits of the other noninvasive techniques, and by using this continuum electromechanics approach noninvasive deep brain stimulation is a possibility.

By altering the permittivity of a material in the presence of an applied electric field a displacement current can be generated, which will thus alter the total current density in the tissue generated by the applied electric field (Melcher, Continuum Electromechanics, 1981). This can be done mechanically by two different means; either by altering what materials are present relative to applied electric field (i.e., mechanically moving material(s) of set permittivities relative to the applied electric field such that the total permittivity in the region of the applied field changes with time) or by mechanically altering the characteristics of the material such that its dipole charge distribution is altered (Melcher, Continuum Electromechanics, 1981).

In the second case one must look at the material. One could examine the tissue in terms of its polarization charge density and the total current within the material where the total

$$current = \frac{\partial \vec{D}}{\partial t} + ∇ \times ( \vec{P} \times \vec{v} ) + {\vec{J}}_{u} .$$

Where, D is the electric displacement, P is the polarization density, v is the velocity of the material, and Jᵤ is the free charge current (represented by σE for free charge neutral biological material). The displacement current

$$\frac{\partial \vec{D}}{\partial t}$$

is equal to

$$\frac{\partial {ɛ}_{0} \vec{E}}{\partial t} + \frac{\partial \vec{P}}{\partial t} ,$$

or

$$ɛ \frac{\partial \vec{E}}{\partial t} + \vec{E} \frac{\partial ɛ}{\partial t}$$

depending on the choice of notation, where P is equal (∈−∈₀)E. P is defined as by the net sum of the dipole effects within a material, equal to nqd where n is the number of dipoles, q the charge of the dipoles, and d the vector distance between the charges, where in most common dielectrics polarization results from the effects of an applied electric field. When using the notation accounting for the polarization density the total current in the material can be written

$$as = \frac{\partial {ɛ}_{0} \vec{E}}{\partial t} + {\vec{J}}_{p} + {\vec{J}}_{u}$$

where the polarization current density, Jₚ, is equal to

$$\frac{\partial \vec{P}}{\partial t} + ∇ \times ( \vec{P} \times \vec{v} ) .$$

Thus when the material is moving relative to the polarization vector or the polarization vector changing relative to time (i.e., the permittivity is changing) a current will be generated. These fundamental physics of continuum electromechanics are reviewed in (Melcher, Continuum Electromechanics, 1981).

In order to capture these effects, MRI derived finite element models (FEM) of the human head was developed using the Ansoft 3D Field Simulator software package to model the base electromagnetic component of the stimulating fields (Wagner, Zahn, Grodzinsky and Pascual-Leone, Three-dimensional head model simulation of transcranial magnetic stimulation, IEEE Trans Biomed Eng, 51, (9), 1586-98, 2004), (Wagner, Valero-Cabre and Pascual-Leone, Noninvasive Human Brain Stimulation, Annu Rev Biomed Eng, 2007), (Wagner, Fregni, Fecteau, Grodzinsky, Zahn and Pascual-Leone, Transcranial direct current stimulation: a computer-based human model study, Neuroimage, 35, (3), 1113-24, 2007), (Ansoft, Maxwell, 2005), (Wagner, Fregni, Eden, Ramos-Estebanez, Grodzinsky, Zahn and Pascual-Leone, Transcranial magnetic stimulation and stroke: a computer-based human model study, Neuroimage, 30, (3), 857-70, 2006). The MRI images were segmented to model tissues in the FEM space, assigning the appropriate electromagnetic conductivity and permittivity to each tissue (see above for impedances and references below for other property characteristics) and guiding the mesh generation based on the MRI derived tissue boundaries, the process of which is detailed in (Wagner, Zahn, Grodzinsky and Pascual-Leone, Three-dimensional head model simulation of transcranial magnetic stimulation, IEEE Trans Biomed Eng, 51, (9), 1586-98, 2004); in the reported figures the results correspond to the ‘measured’ impedance model in the above section. The Ansoft FEM solver was set to solve for the electric field distributions in terms of the electric potential (φ), by solving the equation: ∇·(σEₛ)=∇·(σ∇φ)=0, where σ is the permittivity of each tissue in the head system and Eₛ is the base source electrical field (for more details on the solution process see (Wagner, Zahn, Grodzinsky and Pascual-Leone, Three-dimensional head model simulation of transcranial magnetic stimulation, IEEE Trans Biomed Eng, 51, (9), 1586-98, 2004), (Ansoft, Maxwell, 2005), (Komissarow, Rollnik, Bogdanova, Krampfl, Khabirov, Kossev, Dengler and Bufler, Triple stimulation technique (TST) in amyotrophic lateral sclerosis, Clin Neurophysiol, 115, (2), 356-60, 2004)). A mechanical solution was solved in a similar manner, but via a finite difference time domain (FDTD) solver developed to determine the acoustic propagations through a simulated head system, solving for the Westervelt equation:

$${∇}^{2} p - \frac{1}{{c}^{2}} \frac{{\partial }^{2} p}{\partial {t}^{2}} + \frac{δ}{{c}^{4}} \frac{{\partial }^{3} p}{\partial {t}^{3}} + \frac{β}{ρ {c}^{4}} [ p \frac{{\partial }^{2} p}{\partial {t}^{2}} + {( \frac{\partial p}{\partial t} )}^{2} ] - ∇ p \cdot ∇ ( ln p ) = 0$$

where p is pressure, and c is the speed of sound, δ is acoustic diffusivity, β is the coefficient of the nonlinearity, and ρ is the density of the respective tissues.
(for more details on the solution process see (Connor and Hynynen, Patterns of Thermal Deposition in the Skull During Transcranial Focused Ultrasound Surgery, IEEE Trans Biomed Eng, 51, (10), 1693-1706, 2004)). The output of the two models was fed into an Excel and coupled with a tissue/field perturbation model (Hole and Ditchi, Non-destructive Methods for Space Charge Distribution Measurements: What are the Differences?, IEEE EMBS, 10, (4), 670-677, 2003) to determine field perturbations and changes in bulk permittivity, thus ultimately calculating the current density distributions in the brain during stimulation (where J=σE+∂(∈E)/∂t, J is the current in the tissue, σ the tissue conductivity, E the total field (i.e., source plus perturbation field), and ∈ is the tissue permittivity). The models could be further coupled by feeding the output of the two models into Matlab and coupled with a tissue/field perturbation model [64] and a hybrid Hinch/Fixman inspired model of dielectric enhancement [65-67, 69, 74] to determine field perturbations and changes in bulk permittivity, thus ultimately calculating the current density distributions in the brain during stimulation (where J=σE+∂(∈E)/∂t, J is the current in the tissue, σ the tissue conductivity, E the total electric field (i.e., source plus perturbation field), and ∈ is the tissue permittivity). In this present example, filtering was analyzed initially at the level of the electromagnetic field, and then on a second level via the coupling of the electromechanical fields (a third level of filtering could have been pursued in the mechanical model, but a simplified mechanical solution was pursued in the example).

It should also be noted that the bulk tissue fields can be determined based on the assumption that the continuum electrical effects can be decoupled from mechanical effects on scales greater than expected mechanical perturbation, which can be justified from brain tissue electrorestriction studies and arguments of scale (Spiegel, Ali, Peoples and Joines, Measurement of small mechanical vibrations of brain tissue exposed to extremely-low-frequency electric fields, Bioelectromagnetics, 7, (3), 295-306, 1986), (Wobschall, Bilayer Membrane Elasticity and Dynamic Response, Journal of Colloid and Interface Science, 36, (3), 385-396, 1971), (Wobschall, Voltage Dependence of Bilayer Membrane Capacitance, Journal of Colloid and Interface Science, 40, (3), 417-423, 1972), (Deen, Analysis of Transport Phenomena, 597, 1998). Now, when determining their interaction at the local level (i.e., determining the perturbation on the electric field component at the level of the neural membrane where the mechanical fields are at their maximum strength) this assumption cannot be made, and the fields must be coupled, where the perturbation of the electromagnetic components due the mechanical field can be determined as follows:

∇·((∈+δ∈)(E+δE)=(ρ+δp) (4)

where δ∈ would be equal to the perturbation in local permittivity (such as the permittivity of a cell membrane and/or fluids surrounding (or inside) a cell) due the mechanical field, δE would be equal to the perturbation in the electric field due the mechanical field, and δp would be the perturbation in charge density due to the mechanical field.

### EMS Field Modeling Results

### Mechanical Field:

A field model for a 1 MHz×64 mm transducer was implemented, it was the product of the numerical FDTD simulation of propagation of the initial transient from a focused ultrasound device ran until it reached a continuous wave behavior. This allowed us to demonstrated the predicted mechanical field shape, how it is formed (in time and space), and magnitude in the modeled space. The pressure waves were modeled to indicate the local instantaneous pressure.

### Electrical Field:

The electrical model that we developed is similar to the work we developed in Example 1, but herein for tDCS (broad electrodes, low intensity currents, herein with a 9 cm̂2 area) with a DC field using a Laplacian type solution method (i.e., similar to the DBS methods but at DC, the DBS models spanned multiple frequencies—we implemented the 10 HZ frequency tissue parameters to represent the DC impedances as these were the closest measurement taken in Example 1, and similar to other DC tissue values in the literature). Ultimately the electric field can be made to penetrate deeper into the tissue with broader (i.e., larger surface area) electrodes, and this suggests a number of electrode schemes for maximizing depth. The base electrical currents are proportionately related to the source intensity, herein demonstrated at relative magnitudes (to compare tDCS results to EMS), but can be adjusted accordingly just based on the electric field driving intensity.

### Coupled Model:

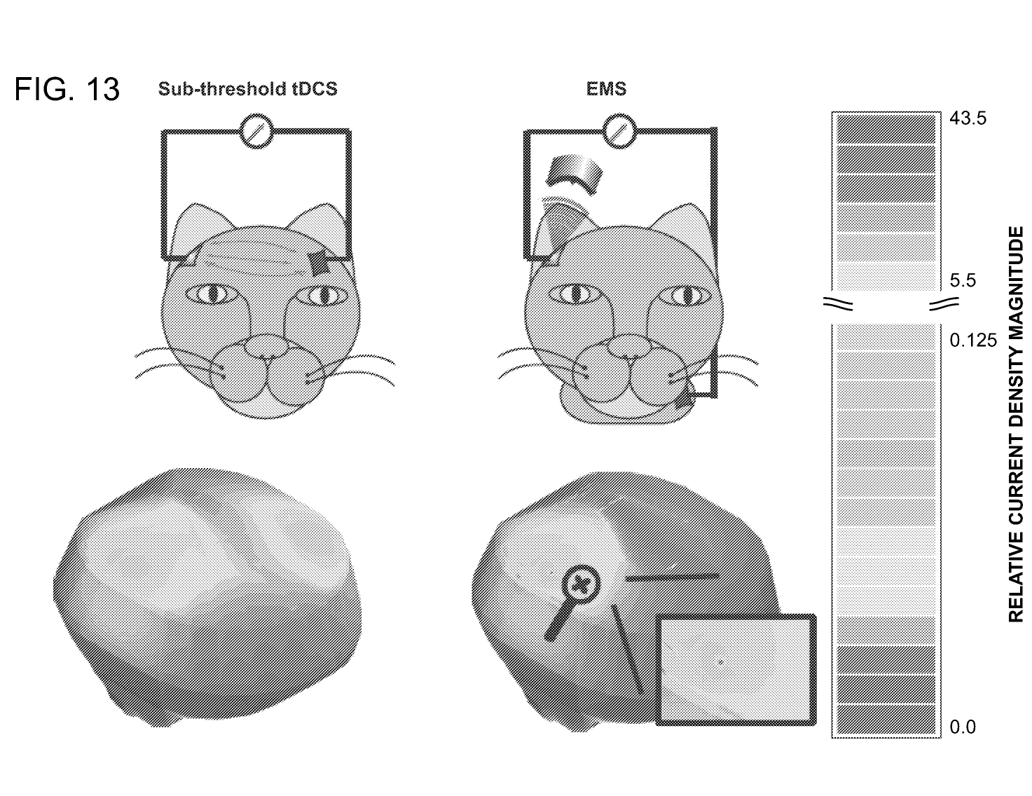

In FIG. 13 a model of coupled electrical and mechanical fields, in terms of their electrical impact on the tissue is demonstrated, with a side-by-side comparison of tDCS (no mechanical field impact) and EMS (tDCS and mechanical fields coupled) is displayed. We modeled the electromagnetic and mechanical field distributions generated during EMS with computational FEM and FDTD models. The models were coupled through a continuum field model of electromechanical interaction (Hole and Ditchi, Non-destructive Methods for Space Charge Distribution Measurements: What are the Differences?, IEEE EMBS, 10, (4), 670-677, 2003) to determine the expected currents generated during EMS. The results demonstrated EMS current density magnitudes ranging on scale from those generated during tDCS to in the range of DBS, with improved focality compared to other noninvasive modalities, subcortical current maxima with appropriate source parameters (although not demonstrated in the current figure, surface stimulation is modeled), and penetration depths surpassing all current noninvasive methods (Wagner, Valero-Cabre and Pascual-Leone, Noninvasive Human Brain Stimulation, Annu Rev Biomed Eng, 2007). See FIG. 13 for a simplified surface example. EMS demonstrates a significant increase in current density magnitude and focality compared to tDCS, for stimulation parameters modeled here (using similar tissue electromagnetic values, note EMS graphical figure generated via graphical modification of FEM electrical model as guided via Matlab/Excel FDTD model results). The calculation is also dependent on how the coupling equation: J=σ_{dc}E+σ_{ω}δE+∈_{ω}jω(δE)+Ejω∈_{(change)dc}+δEjω∈_{(change)ω} is populated, here it is demonstrated at sinusoidal steady assuming an ˜1 MHz steady state frequency, and sub-nano to nanometer level mechanical perturbations from the acoustic field. Although not explicitly demonstrated on the figure, the boost is almost entirely from displacement currents. These models were tested with limited stimulation variables and modeled with a focal EMS transducer.

In terms of depth, EMS mechanical fields are capable of deep penetration, and based on modeling work it is anticipated that broad electrodes, such as a single monopole shaped cap to cover the head, with specialized ground electrodes (such as one in the base of the mouth) could allow stimulation of regions never before reached with a noninvasive stimulator. EMS is the only electromagnetic technique that can generate current density maxima below the brain surface. In terms of focality, the modeling again predicts superiority over the other techniques, and areas of maximum cortical effect up to 2-3 orders of magnitude less than seen with TMS and tDCS.

What is claimed is:

1. A method for stimulating tissue, the method comprising:
analyzing at least one filtering property of a region of at least one tissue; and
providing a dose of energy to the at least one region of tissue based upon results of the analyzing step.

2. The method according to claim 1, wherein the filtering property is selected from the group consisting of: anatomy of the tissue, electromagnetic properties of the tissue, cellular distribution in the tissue, mechanical properties of the tissue, thermodynamic properties of the tissue, chemical distrubtions in the tissue, optical properties of the tissue, and a combination thereof.

3. The method according to claim 1, wherein the stimulation is mechanical stimulation.

4. The method according to claim 2, wherein the mechanical stimulation is ultrasound stimulation.

5. The method according to claim 4, wherein the ultrasound stimulation is focused by a focusing element.

6. The method according to claim 2, wherein mechanical stimulation is in combination with an additional type of stimulation.

7. The method according to claim 6, wherein the additional type of stimulation is selected from the group consisting of: chemical, optical, electromagnetic, and thermal.

8. The method according to claim 1, wherein stimulation comprises a combination of an electric field and a mechanical field.

9. The method according to claim 8, wherein the mechanical field is generated by an ultrasound device.

10. The method according to claim 8, wherein the electric field is pulsed.

11. The method according to claim 8, wherein the electric field is time varying.

12. The method according to claim 8, wherein the electric field is pulsed a plurality of time, and each pulse may be for a different length of time.

13. The method according to claim 8, wherein the electric field is time invariant.

14. The method according to claim 8, wherein the mechanical field is pulsed.

15. The method according to claim 8, wherein the mechanical field is time varying.

16. The method according to claim 8, wherein the mechanical field is pulsed a plurality of time, and each pulse may be for a different length of time.

17. The method according to claim 1, wherein stimulation is applied to a structure or multiple structures within the brain or the nervous system selected from the group consisting of: dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord.

18. The method according to claim 1, wherein the tissue is neural tissue.

19. The method according to claim 18, wherein the affect of the stimulation alters neural function past the duration of stimulation.

20. The method according to claim 1, wherein the stimulation is selected from the group consisting of: electrical, mechanical, thermal, optical, and a combination thereof.

21. A method for stimulating tissue, the method comprising: providing a dose of energy to a region of tissue, wherein the dose provided is based upon at least one filtering property of the region of tissue.

22. A method for stimulating tissue, the method comprising:
analyzing at least one filtering property of a region of tissue;
providing a dose of electrical energy to the region of tissue; and
providing a dose of mechanical energy to the region of tissue, wherein the combined dose of energy provided to the tissue is based upon results of the analyzing step.

23. A method for stimulating tissue, the method comprising:
providing a noninvasive transcranial neural stimulator; and
using the stimulator to stimulate a region of tissue, wherein a dose of energy provided to the region of tissue is based upon at least one filtering property of the region of tissue.
